(19) United States
(12) Patent Application Publication
(10) Pub. No.: US 2023/0203185 A1
(43) Pub. Date: Jun. 29, 2023

(54) CD 163 ANTIBODIES OR BINDING PROTEINS

(71) Applicant: ECO ANIMAL HEALTH LTD., New Malden (GB)

(72) Inventors: Charles OWEN, Uckfield (GB); Hafid Abdelaali BENCHAOUI, New Malden (GB); Christine TAIT-BURKARD, Roslin (GB)

(73) Assignee: ECO ANIMAL HEALTH LTD., New Malden (GB)

(21) Appl. No.: 17/788,539

(22) PCT Filed: Dec. 24, 2020
(86) PCT No.: PCT/GB2020/053370

§ 371 Date: Jun. 23, 2022

(30) Foreign Application Priority Data
Dec. 24, 2019 (GB) 1919294.7

(51) Int. Cl. C07K 16/28

(52) U.S. Cl. CPC C07K 16/2896; A61K 2039/505

(57) ABSTRACT

The present disclosure provides a monoclonal antibody which binds to porcine CD163 for use in the treatment or prevention of Porcine Reproductive and Respiratory Syndrome (PRRS) virus infection in a pig. Preferred antibodies comprise an antigen binding domain which binds to porcine CD163, said antigen binding domain comprising at least one heavy chain variable region which comprises three complementarity determining regions (CDRs), wherein said heavy chain variable region comprises a variable heavy (VH) CDR2 that comprises the amino acid sequence XYAD or XYAE or XYAN, in which X can be any amino acid. Nucleic acid molecules, expression vectors and compositions are also provided.

This invention relates generally to the field of binding proteins which bind to CD163 (Cluster of Differentiation 163), in particular antibodies, and in particular binding proteins and antibodies that bind to porcine CD163. Such anti-CD163 binding proteins and antibodies have therapeutic and protective uses, such as in the treatment or prevention of infections such as Porcine Reproductive and Respiratory Syndrome (PRRS) virus infections, for example reducing their incidence and severity. Binding protein and antibody-based compositions, methods and kits are also provided.

Porcine Reproductive and Respiratory Syndrome (PRRS) is one of the most devastating viral pig diseases worldwide and causes huge economic losses to the pig farming industry. The causative agent is PRRS virus (PRRSV), an enveloped RNA virus classified in the family Arteriviridae within the order Nidovirales. PRRSV has a restricted host and cell tropism, with porcine alveolar macrophages (PAMs) as important target cells. Clinical symptoms are diverse, but include respiratory distress and respiratory disease in young pigs and piglets, late-term abortion and still-births in gilts and sows, foetal reabsorbtion in early pregnancy and reduced growth in finishing pigs. Due to reduction or loss of pregnancies, death in young piglets, and decreased growth rates in all PRRSV infected pigs, it is estimated that more than $650 m are lost annually to pork producers in the U.S. alone.

All currently known PRRSV isolates fall into one of two genotypes (or species), type 1 (PRRSV-1) or type 2 (PRRSV-2), which have only about 60% identity at the nucleotide level, although they both cause long-term infections and produce similar clinical signs. Genotype 1 originated in Europe and tends to be found in European PRRSV isolates or strains, whereas genotype 2 originated in North America and tends to be found in Asian or American isolates or strains (see review by Stoian and Rowland, 2019, Vet. Sci., 6, 9). Within each genotype, there is significant diversity with a large number of strains identified, including new highly pathogenic strains that have emerged since 2006, particularly in China and Vietnam. Similar highly pathogenic strains have also emerged elsewhere, stretching from the Malaysian peninsula to southern Russia and these present a growing threat to the pig population (An et al., 2011, Emerging Infect Dis 17(9):1782). In China alone, more than 20 million pigs were culled because of PRRS virus infection annually in 2006 and 2007 (An et al., 2010, Emerging Infect Dis 16(2):365). More recently, case reports of virulent strains causing outbreaks in Europe indicate the growing emergence of PRRS virus as a threat (Sinn et al., 2016, Porcine Health Management (2):28).

The scavenger receptor CD163 is a key entry mediator for PRRSV infection and thus has a key role in PRRSV infection. CD163 is a 130 kDa type I transmembrane protein which has a signal peptide followed by nine scavenger-receptor cysteine rich (SRCR) domains, each approximately 100 amino acids in length, with a 35 amino-acid proline-serine-threonine (PST)-rich region separating SRCR domain 6 (SRCR6) and SRCR7. A second PST-rich region connects SRCR9 with the transmembrane domain and a short cytoplasmic tail, which contains a functional internalization motif. Surface expression of CD163 is restricted to cells of the monocyte-macrophage lineage. The SRCR5 domain of CD163 has been identified to play a significant role in order for infection of porcine alveolar macrophages by PRRSV to occur (Gorp et al., 2010, J. of Virology, March, 3101-3105).

The precise mechanism of PRRSV infection is unknown. However, as part of this mechanism PRRSV is believed to enter the endosomal compartment of cells, in which an interaction between CD163 and the GP2-GP3-GP4 heterotrimer of the PRRSV mediates uncoating of the virus and the release of the viral genome into the cytoplasm.

One proposed treatment option for PRRSV includes some kind of genetic knockout or gene editing of CD163, in order to make pigs resistant to PRRSV infection, and then breeding these pigs to propagate the genetic modification (Burkard et al., 2017, PLOS Pathogens 13(2):e1006206). Although this has been shown to work quite effectively, this treatment will be complex and time consuming in terms of being able to treat a significant proportion of the porcine population. In addition, and importantly, there is significant resistance in many markets to techniques involving the genetic modification of animals, for example when it comes to the desirability of animal products produced from such animals.

The most common medical intervention used to limit the economic impact of PRRS is vaccination. Vaccines are routinely used in all geographies where the disease is prevalent. Two types of vaccines are routinely used, either killed virus vaccines (virins) or (in the majority of cases) modified live vaccines (MLV). However, currently vaccines are only partially effective and add most value when deployed within an integrated approach to disease management where concurrent biosecurity and husbandry decisions are closely aligned. The reasons behind lack of vaccine efficacy are complex but the high genetic diversity of the PRRSV population coupled with the biology of the virus (tropism for alveolar macrophages and high mutability) are such that the best results are seen when the vaccine strain and the circulating strain are closely matched in terms of immunogenicity (reviewed by Nan et al., 2017, Front. Immunol. 8: 1635). Additionally, live vaccine strains can recombine with field strains to produce new field strains which may be pathogenic.

There are currently no anti-viral treatment options for PRRSV infections.

Thus, there is a clear need for alternative and preferably improved treatment and prevention options for PRRSV infection (or other CD163 mediated infections) which can readily be used to treat or prevent infection in significant numbers of animals.

The present invention provides one such alternative therapeutic or preventative option in the form of binding proteins and antibodies directed to porcine CD163, which can act to reduce or prevent PRRSV infection.

Surprisingly, single types of antibody (monoclonal antibodies), which target the same epitope on porcine CD163, as opposed to for example a polyclonal antibody preparation which would target multiple different epitopes on porcine CD163, have been shown to be effective to significantly reduce or prevent PRRSV infection. In addition, a sub-set of these anti-CD163 antibodies have been identified which exhibit differential inhibition of infection with type 1 and/or type 2 PRRSV.

The present inventors have thus provided anti-CD163 antibodies that are able to bind to and inhibit the activity or function of CD163, in particular porcine CD163. Such antibodies (or for example other binding proteins comprising a CD163 antigen binding domain as described herein) can for example inhibit the ability of CD163 to interact with other proteins such as viral proteins, and thereby inhibit the infection of cells such as porcine alveolar macrophages. Such antibodies (or for example other binding proteins comprising a CD163 antigen binding domain as described herein) can conveniently and advantageously be used to treat or prevent infection in pigs, in particular PRRSV infection.

In one embodiment, the present invention provides a binding protein, for example an antibody, for example a monoclonal antibody, which binds to CD163, for example porcine CD163, for use in the treatment or prevention of infection, for example PRRS virus infection or CD163 mediated infection, in a pig. Thus, in particular, the present invention provides a monoclonal antibody which binds to porcine CD163 for use in the treatment or prevention of PRRS virus infection in a pig. However, the antibodies of the present invention can be used in the treatment or prevention of any pathologies of the pig where CD163 is shown to play a role, in which case binding and inhibition of this protein could be a useful therapeutic tool.

As discussed elsewhere herein, preferred antibodies (or binding proteins) of the invention and suitable for use in the therapeutic methods described herein have the ability to bind to the SRCR5 domain of CD163, for example have an epitope in the SRCR5 domain of CD163. In addition, preferred antibodies (or binding proteins) have the ability to inhibit type 1 and/or type 2 PRRSV infection, more preferably type 1 and type 2 PRRSV infection. In addition, some preferred antibodies have the ability to inhibit type 2 PRRSV infection, and preferably have the ability to specifically inhibit type 2 PRRSV infection.

### Ability to Inhibit Type 1 and/or Type 2 PRRSV Infection

### Family 40

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:2), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of GIAWSGRAPYADSVKG (SEQ ID NO:3), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAIRWTTLDAYDY (SEQ ID NO:4), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:10), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AISWSGRAPYADSVKG (SEQ ID NO:11), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAIKWTTLDAYDY (SEQ ID NO:12), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:18), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of GIAWSGRAPYADSVKG (SEQ ID NO:19), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAILWTTPGAYNY (SEQ ID NO:20), or a sequence substantially homologous thereto, wherein said substantially homologous sequence is a sequence containing 1, 2, 3 or 4 amino acid substitutions compared to the given CDR sequence.

In a preferred embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:2),
(ii) a variable heavy (VH) CDR2 that comprises the amino acid sequence of GIAWSGRAPYADSVKG (SEQ ID NO:3), and
(iii) a variable heavy (VH) CDR3 that comprises the amino acid sequence of GEGAIRWTTLDAYDY (SEQ ID NO:4).

In a preferred embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:10),
(ii) a variable heavy (VH) CDR2 that comprises the amino acid sequence of AISWSGRAPYADSVKG (SEQ ID NO:11), and
(iii) a variable heavy (VH) CDR3 that comprises the amino acid sequence of GEGAIKWTTLDAYDY (SEQ ID NO:12).

In a preferred embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:18),
(ii) a variable heavy (VH) CDR2 that comprises the amino acid sequence of GIAWSGRAPYADSVKG (SEQ ID NO:19), and
(iii) a variable heavy (VH) CDR3 that comprises the amino acid sequence of GEGAILWTTPGAYNY (SEQ ID NO:20).

In further embodiments of the present invention, the VH CDR2 has or comprises the amino acid sequence of X₁ I X₃ W S G R A P Y A D S V K G (SEQ ID NO: 73). In these embodiments X₁ or X₃ can be any amino acid. Preferably one or more, most preferably all, of these X residues are selected from the following group: X₁ is G or A, and X₃ is A or S. Thus, a preferred VH CDR2 has or comprises the amino acid sequence of G/A I A/S W S G R A P Y A D S V K G (SEQ ID NO: 74). For example, preferred VH CDR2 sequences of this embodiment have or comprise SEQ ID NOs: 3, 11 or 19.

In further embodiments of the present invention, the VH CDR3 has or comprises an amino acid sequence of G E G A I X₆ W T T X₁₀ X₁₁ A Y X₁₄ Y (SEQ ID NO:75). In these embodiments X₆, X₁₀ X₁₁ and X₁₄ can be any amino acid. Preferably one or more, most preferably all, of these X residues are selected from the following group: X₆ is R or K or L; X₁₀ is L or P; X₁₁ is D or G, and X₁₄ is D or N. Thus, a preferred VH CDR3 has or comprises the amino acid sequence of G E G A I R/K/L W T T UP D/G A Y D/N Y (SEQ ID NO:76). For example, preferred VH CDR3 sequences of this embodiment have or comprise SEQ ID NOs: 4, 12 or 20.

In a further embodiment, the present invention provides an antibody (or binding protein) that comprises:
a VH region that comprises a VH CDR1 of SEQ ID NO:2 or a sequence substantially homologous thereto, wherein said substantially homologous sequence is a sequence containing 1 or 2 (preferably 1), altered amino acids compared with the given CDR sequence, a VH CDR2 of SEQ ID NO:73, and a VH CDR3 of SEQ ID NO:75. In some such embodiments, the VH CDR1 is preferably SEQ ID NO: 2. In some such embodiments, the VH CDR2 is preferably SEQ ID NO: 3, 11 or 19. In some such embodiments, the VH CDR3 is preferably SEQ ID NO: 4, 12 or 20.

In one embodiment, the present invention provides an antibody (or binding protein) that comprises:
a VH region that comprises a VH CDR1 of SEQ ID NO:2 or a sequence substantially homologous thereto, wherein said substantially homologous sequence is a sequence containing 1 or 2 (preferably 1), altered amino acids compared with the given CDR sequence, a VH CDR2 of SEQ ID NO:74, and a VH CDR3 of SEQ ID NO:76. In some such embodiments, the VH CDR1 is preferably SEQ ID NO: 2. In some such embodiments, the VH CDR2 is preferably SEQ ID NO: 3, 11 or 19. In some such embodiments, the VH CDR3 is preferably SEQ ID NO: 4, 12 or 20.

In further embodiments of the invention, antibodies (or binding proteins) comprise:
a VH region that comprises a VH CDR1 of SEQ ID NO:2 or a sequence containing 1 or 2 (preferably 1) altered amino acids compared with the given CDR sequence, a VH CDR2 of SEQ ID NO:73 or a sequence substantially homologous thereto, and a VH CDR3 of SEQ ID NO:75, or a sequence substantially homologous thereto. In such embodiments said substantially homologous sequence is a sequence containing 1, 2, 3 or 4, preferably 1, 2 or 3, preferably 1 or 2 (more preferably 1), altered amino acids compared with a given CDR sequence.

In further embodiments of the invention, antibodies (or binding proteins) comprise:
a VH region that comprises a VH CDR1 of SEQ ID NO:2 or a sequence containing 1 or 2 (preferably 1) altered amino acids compared with the given CDR sequence, a VH CDR2 of SEQ ID NO:74 or a sequence substantially homologous thereto, and a VH CDR3 of SEQ ID NO:76 or a sequence substantially homologous thereto. In such embodiments said substantially homologous sequence is a sequence containing 1, 2, 3 or 4, preferably 1, 2 or 3, preferably 1 or 2 (more preferably 1), altered amino acids compared with a given CDR sequence.

### Family 70

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYSMG (SEQ ID NO:26), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AHRWSGSAYYAEHADSVEG (SEQ ID NO:27), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GVGSAAQYRY (SEQ ID NO:28), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of PGSMG (SEQ ID NO:34), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AHRWSGSAYYADYADSVEG (SEQ ID NO:35), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GVGSAAQYTY (SEQ ID NO:36), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYSMG (SEQ ID NO:42), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AHRWSGSAYYAEHADSVEG (SEQ ID NO:43), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GVGSEAQYRY (SEQ ID NO:44), or a sequence substantially homologous thereto, wherein said substantially homologous sequence is a sequence containing 1, 2, 3 or or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYSMG (SEQ ID NO:26),
(ii) a variable heavy (VH) CDR2 that comprises the amino acid sequence of AHRWSGSAYYAEHADSVEG (SEQ ID NO:27), and
(iii) a variable heavy (VH) CDR3 that comprises the amino acid sequence of GVGSAAQYRY (SEQ ID NO:28).

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of PGSMG (SEQ ID NO:34),
(ii) a variable heavy (VH) CDR2 that comprises the amino acid sequence of AHRWSGSAYYADYADSVEG (SEQ ID NO:35), and
(iii) a variable heavy (VH) CDR3 that comprises the amino acid sequence of GVGSAAQYTY (SEQ ID NO:36).

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYSMG (SEQ ID NO:42),
(ii) a variable heavy (VH) CDR2 that comprises the amino acid sequence of AHRWSGSAYYAEHADSVEG (SEQ ID NO:43), and
(iii) a variable heavy (VH) CDR3 that comprises the amino acid sequence of GVGSEAQYRY (SEQ ID NO:44).

In further embodiments of the present invention, the VH CDR1 has or comprises the amino acid sequence of X₁ X₂ S M G (SEQ ID NO:77). In these embodiments X₁ or X₂ can be any amino acid. Preferably one or more, most preferably all, of these X residues are selected from the following group: X₁ is T or P, and X₂ is Y or G. Thus, a preferred VH CDR1 has or comprises the amino acid sequence of T/P Y/G S M G (SEQ ID NO:78). For example, preferred VH CDR1 sequences of this embodiment have or comprise SEQ ID NOs: 26, 34 or 42.

In further embodiments of the present invention, the VH CDR2 has or comprises the amino acid sequence of A H R W S G S A Y Y A X₁₂ X₁₃ A D S V E G (SEQ ID NO:79). In these embodiments X₁₂ or X₁₃ can be any amino acid. Preferably one or more, most preferably all, of these X residues are selected from the following group: X₁₂ is E or D and X₁₃ is H or Y. Thus, a preferred VH CDR2 has or comprises the amino acid sequence of A H R W S G S A Y Y A E/D H/Y A D S V E G (SEQ ID NO:80). For example, preferred VH CDR2 sequences of this embodiment have or comprise SEQ ID NOs: 27, 35 or 43.

In further embodiments of the present invention, the VH CDR3 has or comprises an amino acid sequence of G V G S X₅ A Q Y X₉ Y (SEQ ID NO:81). In these embodiments X₅ and X₉ can be any amino acid. Preferably one or more, most preferably all, of these X residues are selected from the following group: X₅ is A or E, and X₉ is R or T. Thus, a preferred VH CDR3 has or comprises the amino acid sequence of G V G S A/E A Q Y R/T Y (SEQ ID NO:82). For example, preferred VH CDR3 sequences of this embodiment have or comprise SEQ ID NOs: 28, 36 or 44.

In one embodiment, the present invention provides an antibody (or binding protein) that comprises:
a VH region that comprises a VH CDR1 of SEQ ID NO:77, a VH CDR2 of SEQ ID NO:79, and a VH CDR3 of SEQ ID NO:81. In some such embodiments, the VH CDR1 is preferably SEQ ID NO: 26, 34 or 42. In some such embodiments, the VH CDR2 is preferably SEQ ID NO: 27, 35 or 43. In some such embodiments, the VH CDR3 is preferably SEQ ID NO: 28, 36 or 44.

In one embodiment, the present invention provides an antibody (or binding protein) that comprises:
a VH region that comprises a VH CDR1 of SEQ ID NO:78, a VH CDR2 of SEQ ID NO:80, and a VH CDR3 of SEQ ID NO:82. In some such embodiments, the VH CDR1 is preferably SEQ ID NO: 26, 34 or 42. In some such embodiments, the VH CDR2 is preferably SEQ ID NO: 27, 35 or 43. In some such embodiments, the VH CDR3 is preferably SEQ ID NO: 28, 36 or 44.

In other embodiments of the invention, antibodies (or binding proteins) comprise:
a VH region that comprises a VH CDR1 of SEQ ID NO:77 or a sequence containing 1 or 2 (preferably 1) altered amino acids compared with the given CDR sequence, a VH CDR2 of SEQ ID NO:79 or a sequence substantially homologous thereto, and a VH CDR3 of SEQ ID NO:81, or a sequence substantially homologous thereto. In such embodiments said substantially homologous sequence is a sequence containing 1, 2, 3 or 4, preferably 1, 2 or 3, preferably 1 or 2 (more preferably 1), altered amino acids compared with a given CDR sequence.

In other embodiments of the invention, antibodies (or binding proteins) comprise:
a VH region that comprises a VH CDR1 of SEQ ID NO:78 or a sequence containing 1 or 2 (preferably 1) altered amino acids compared with the given CDR sequence, a VH CDR2 of SEQ ID NO:80 or a sequence substantially homologous thereto, and a VH CDR3 of SEQ ID NO:82 or a sequence substantially homologous thereto. In such embodiments said substantially homologous sequence is a sequence containing 1, 2, 3 or 4, preferably 1, 2 or 3, preferably 1 or 2 (more preferably 1), altered amino acids compared with a given CDR sequence.

In embodiments of the invention where one or more of the CDR sequences contain an Xx residue (or another type of alternative residue as defined herein), then CDRs with sequences which are substantially homologous thereto containing 1, 2, 3 or 4, preferably 1, 2 or 3 (more preferably 1 or 2, or 1), altered amino acids or amino acid substitutions compared with a given CDR sequence are also encompassed by the invention. In some such embodiments said alterations or substitutions in amino acid residues can include one or more of the Xₓ residues or can be at residues other than the Xₓ residues. In other such embodiments said alterations are in a mixture of the Xₓ residues and the non-X_{X} residues.

### Clone 150(#15)

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of SYSMG (SEQ ID NO:50), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AITWNGYITNYADSVKG (SEQ ID NO:51), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of TTFSTTSPISRTYNY (SEQ ID NO:52), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of SYSMG (SEQ ID NO:50),
(ii) a variable heavy (VH) CDR2 that comprises the amino acid sequence of AITWNGYITNYADSVKG (SEQ ID NO:51), and
(iii) a variable heavy (VH) CDR3 that comprises the amino acid sequence of TTFSTTSPISRTYNY (SEQ ID NO:52).

### Clone 70(#23)

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYAMG (SEQ ID NO:58), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of IISFGGTFYADSVKG (SEQ ID NO:59), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GRTLSKRADSYAS (SEQ ID NO:60), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYAMG (SEQ ID NO:58),
(ii) a variable heavy (VH) CDR2 that comprises the amino acid sequence of IISFGGTFYADSVKG (SEQ ID NO:59), and
(iii) a variable heavy (VH) CDR3 that comprises the amino acid sequence of GRTLSKRADSYAS (SEQ ID NO:60).

### Clone 144(#1)

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of MYAMS (SEQ ID NO:66), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AINTSGRYSRYADSVKG (SEQ ID NO:67), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of TDKGNWALAMSYDY (SEQ ID NO:68), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of MYAMS (SEQ ID NO:66),
(ii) a variable heavy (VH) CDR2 that comprises the amino acid sequence of AINTSGRYSRYADSVKG (SEQ ID NO:67), and
(iii) a variable heavy (VH) CDR3 that comprises the amino acid sequence of TDKGNWALAMSYDY (SEQ ID NO:68).

All the antibodies (or binding proteins) described in the above section have the ability to inhibit type 1 and type 2 PRRSV infection, and thus can be used in the treatment or prevention of type 1 and/or type 2 PRRSV infection.

### Ability to Inhibit Type 2 PRRSV Infection

As mentioned above, other anti-CD163 antibodies and binding proteins of the invention have the ability to inhibit type 2 PRRSV infection, and preferably specifically (or only, or preferentially) inhibit type 2 PRRSV infection, for example inhibit type 2 PRRSV infection but do not inhibit (or not significantly inhibit) type 1 PRRSV infection. Thus, a further embodiment of the invention provides antibodies (or binding proteins) that can specifically inhibit type 2 PRRSV infection. Examples of such “type 2” antibodies or binding proteins are described below. In other preferred embodiments these “type 2” antibodies and binding proteins which can inhibit type 2 PRRSV infection can be used in combination with the antibodies described above, which can inhibit type 1 and/or type 2 PRRSV infection, and preferably inhibit type 1, or type 1 and type 2 PRRSV infection.

### Clone 57(#11)

Thus, in a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of VYGTG (SEQ ID NO:84), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of GISGTTGSTLYADSVKG (SEQ ID NO:85), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GGRVYITTSSWAY (SEQ ID NO:86), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of VYGTG (SEQ ID NO:84),
(ii) a variable heavy (VH) CDR2 that comprises the amino acid sequence of GISGTTGSTLYADSVKG (SEQ ID NO:85), and
(iii) a variable heavy (VH) CDR3 that comprises the amino acid sequence of GGRVYITTSSWAY (SEQ ID NO:86).

### Clone 41 (#12)

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYAMG (SEQ ID NO:92), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AIAWSTGSTYYANSVKG (SEQ ID NO:93), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of ETRYCSGFGCLDPRTYGS (SEQ ID NO:94), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYAMG (SEQ ID NO:92),
(ii) a variable heavy (VH) CDR2 that comprises the amino acid sequence of AIAWSTGSTYYANSVKG (SEQ ID NO:93), and
(iii) a variable heavy (VH) CDR3 that comprises the amino acid sequence of ETRYCSGFGCLDPRTYGS (SEQ ID NO:94).

### Clone 171 (#14)

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TDTMA (SEQ ID NO:100), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of GIGRSGGSIYYADAVKG (SEQ ID NO:101), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of RQRIGLVVGALGYDY (SEQ ID NO:102), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TDTMA (SEQ ID NO:100),
(ii) a variable heavy (VH) CDR2 that comprises the amino acid sequence of GIGRSGGSIYYADAVKG (SEQ ID NO:101), and
(iii) a variable heavy (VH) CDR3 that comprises the amino acid sequence of RQRIGLVVGALGYDY (SEQ ID NO:102).

### Clone 29(#17)

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:108), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of CINSITSNTYYADSVKG (SEQ ID NO:109), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of DSGLFSGSSCLKYRAMRFGS (SEQ ID NO:110), or a sequence substantially homologous thereto, wherein said substantially homologous sequence is a sequence containing 1, 2, 3 or 4 amino acid substitutions compared to the given CDR sequence.

In a further embodiment, the present invention provides a binding protein, for example an antibody, comprising an antigen binding domain which binds to CD163, for example porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:108),
(ii) a variable heavy (VH) CDR2 that comprises the amino acid sequence of CINSITSNTYYADSVKG (SEQ ID NO:109), and
(iii) a variable heavy (VH) CDR3 that comprises the amino acid sequence of DSGLFSGSSCLKYRAMRFGS (SEQ ID NO:110).

### Other Embodiments

Certain preferred embodiments of the invention provide an antibody (or binding protein) which binds to CD163, for example porcine CD163, comprising a VH domain that has the amino acid sequence of SEQ ID NO: 1, 9, 17, 25, 33, 41, 49, 57 or 65, or a sequence substantially homologous thereto. In some embodiments, such antibodies (or binding proteins) also comprise a VL domain which comprises up to three light chain CDRs, and preferably three light chain CDRs.

In a preferred embodiment the present invention provides an antibody (or binding protein) which binds to CD163, for example porcine CD163, comprising a VH domain that has the amino acid sequence of SEQ ID NO: 1, 9, 17, 25, 33, 41, 49, 57 or 65, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs.

In a preferred embodiment, the present invention provides an antibody (or binding protein), which binds to CD163, for example porcine CD163, comprising a VH domain that has the amino acid sequence of SEQ ID NO: 1, 9, 17, 25, 33, 41, 49, 57 or 65. In some embodiments, such antibodies (or binding proteins) also comprise a VL domain which comprises up to three light chain CDRs, and preferably three light chain CDRs.

Certain preferred embodiments of the invention provide an antibody (or binding protein) which binds to CD163, for example porcine CD163,comprising a VH domain that has the amino acid sequence of SEQ ID NO: 83, 91, 99 or 107, or a sequence substantially homologous thereto. In some embodiments, such antibodies (or binding proteins) also comprise a VL domain which comprises up to three light chain CDRs, and preferably three light chain CDRs.

In a preferred embodiment the present invention provides an antibody (or binding protein) which binds to CD163, for example porcine CD163, comprising a VH domain that has the amino acid sequence of SEQ ID NO: 83, 91, 99 or 107, or a sequence having at least 80% sequence identity thereto (e.g. at least 85%, 90%, 95% or 98% identity). In some embodiments, such antibodies (or binding proteins) also comprise a VL domain which comprises up to three light chain CDRs, and preferably three light chain CDRs.

In a preferred embodiment, the present invention provides an antibody (or binding protein) which binds to CD163, for example porcine CD163, comprising a VH domain that has the amino acid sequence of SEQ ID NO: 83, 91, 99 or 107. In some embodiments, such antibodies (or binding proteins) also comprise a VL domain which comprises up to three light chain CDRs, and preferably three light chain CDRs.

Other preferred embodiments are immunoglobulin (Ig) forms, e.g. IgG forms, or forms containing all or part of an immunoglobulin constant region, e.g. an IgG constant region, of the various antibodies (or binding proteins) defined herein, for example full length Ig or IgG forms. It is of course understood that full IgG antibodies will typically comprise two substantially identical heavy chains and two substantially identical light chains. Preferred forms containing part of an immunoglobulin constant region are forms containing an Fc region or domain, for example Fc fusions. Such Fc regions or domains are known in the art and generally comprise CH2 and CH3 domains of antibody heavy chains, which associate to form a homodimer. These regions can be derived from any appropriate source or species, e.g. a source or species different from the host species used to generate the antibodies, e.g. by immunization, or a source or species different from where the antibodies are derived, but preferably correspond to or are derived from porcine Fc regions or domains. As such Fc regions are homodimeric (or form homodimers), they can conveniently be used to dimerise two polypeptide chains. Thus, by linking or fusing one or more single domain antibodies (e.g. VHH antibodies) of the invention to each chain of an Fc region, when the two chains of the Fc region dimerise they can be used to provide multiple copies of single domain antibodies (e.g. VHH antibodies) of the invention in a single construct or molecule. If more than one single domain antibody (e.g. VHH antibody) of the invention is linked or fused to each chain of an Fc region in sequence then these antibodies can be the same antibody (e.g. two or more copies of the same VHH can be provided on each chain) or different antibodies. Thus, for example, the Fc fusion can be used to provide constructs comprising more than one copies of identical single domain antibodies of the invention or more than one copies of different single domain antibodies of the invention. As such constructs generally contain more than one copy of the same antibody (e.g. more than one copy of one single domain antibody or VHH antibody or more than one copy of multiple different single domain antibody or VHH antibody) of the invention, such constructs may show improved binding of CD163, for example due to an avidity effect.

Binding proteins, e.g. antibodies, based on the 49(#18), 47(#19), 48(#20), 76(#2), 77(#16), 78(#8), 150(#15), 70(#23) or 144(#1) antibody sequences set forth in Tables A, B, C, D, E, F, G, H or I are preferred. The invention is exemplified by monoclonal antibodies which are VHH antibodies (single domain antibodies), sequences of which are shown in Tables A, B, C, D, E, F, G, H and I herein. The VH CDR domains and VH domains of each of these VHH antibodies are shown in Tables A to I herein. Antibodies (or binding proteins) comprising these sets of VH CDR domains, or VH domains, or IgG sequences comprising such domains (or sequences substantially homologous thereto) are preferred embodiments of the invention.

In addition, binding proteins, e.g. antibodies based on the 57(#11), 41 (#12), 171(#14), 29(#17) antibody sequences set forth in Tables 1, 2, 3 or 4 are preferred. The invention is exemplified by monoclonal antibodies which are VHH antibodies (single domain antibodies), sequences of which are shown in Tables 1, 2, 3 and 4 herein. The VH CDR domains and VH domains of each of these VHH antibodies are shown in Tables 1, 2, 3 and 4 herein. Antibodies (or binding proteins) comprising these sets of VH CDR domains, or VH domains, or IgG sequences comprising such domains (or sequences substantially homologous thereto) are preferred embodiments of the invention.

Certain examples of substantially homologous sequences are sequences that have at least 60% or 65% identity to the amino acid sequences disclosed. In certain embodiments, the antibodies (or binding proteins) of the invention comprise at least one heavy chain variable region that includes an amino acid sequence region of at least about 60%, 65%, 70% or 75%, more preferably at least about 80%, more preferably at least about 85%, more preferably at least about 90% or 95% and most preferably at least about 97%, 98% or 99% amino acid sequence identity to the amino acid sequence of SEQ ID NO: 1, 9, 17, 25, 33, 41, 49, 57 or 65.

Other certain examples of substantially homologous sequences are sequences that have at least 60% or 65% identity to the amino acid sequences disclosed. In certain embodiments, the antibodies (or binding proteins) of the invention comprise at least one heavy chain variable region that includes an amino acid sequence region of at least about 60%, 65%, 70% or 75%, more preferably at least about 80%, more preferably at least about 85%, more preferably at least about 90% or 95% and most preferably at least about 97%, 98% or 99% amino acid sequence identity to the amino acid sequence of SEQ ID NO: 83, 91, 99 or 107.

Other preferred examples of substantially homologous sequences are sequences containing conservative amino acid substitutions of the amino acid sequences disclosed.

Other preferred examples of substantially homologous sequences are sequences containing 1, 2, 3 or 4, preferably 1, 2 or 3, preferably 1 or 2 (more preferably 1), altered amino acids in one or more of the CDR regions or one or more of the FR regions disclosed. Such alterations might be conserved or non-conserved amino acid substitutions, or a mixture thereof.

In such embodiments, preferred alterations are conservative amino acid substitutions.

In all embodiments, binding proteins, e.g. antibodies, containing substantially homologous sequences retain the ability to bind to CD163, e.g. porcine CD163. Preferably, binding proteins, e.g. antibodies, containing substantially homologous sequences retain one or more (preferably all) of the other properties described herein in relation to the 49(#18), 47(#19), 48(#20), 76(#2), 77(#16), 78(#8), 150(#15), 70(#23) or 144(#1) antibodies.

In all embodiments, binding proteins, e.g. antibodies, containing substantially homologous sequences retain the ability to bind to CD163, e.g. porcine CD163. Preferably, binding proteins, e.g. antibodies, containing substantially homologous sequences retain one or more (preferably all) of the other properties described herein in relation to the 57(#11), 41(#12), 171(#14) or 29(#17) antibodies.

Further examples of substantially homologous amino acid sequences in accordance with the present invention are described elsewhere herein.

The CDRs of the antibodies (or binding proteins) of the invention are preferably separated by appropriate framework regions such as those found in naturally occurring antibodies and/or effective engineered antibodies. Thus, the V_{H} (e.g. VHH), V_{L} and individual CDR sequences of the invention are preferably provided within or incorporated into an appropriate framework or scaffold to enable antigen (here CD163) binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions. Alternatively, non-antibody scaffolds or frameworks, e.g. T cell receptor frameworks can be used.

Appropriate sequences that can be used for framework regions are well known and documented in the art and any of these may be used. Preferred sequences for framework regions are one or more of the framework regions making up the VHH antibodies of the invention, preferably one or more of the framework regions of the 49(#18), 47(#19), 48(#20), 76(#2), 77(#16), 78(#8), 150(#15), 70(#23), or 144(#1) VHH antibodies, as disclosed in Tables A, B, C, D, E, F, G, H and I, or framework regions substantially homologous thereto, and in particular framework regions that allow the maintenance of antigen specificity, for example framework regions that result in substantially the same or the same 3D structure of the antibody.

Other preferred sequences for framework regions, in particular for the “type 2” antibodies of the invention, are one or more of the framework regions making up the VHH antibodies of the invention, preferably one or more of the framework regions of the 57(#11), 41(#12), 171(#14), or 29(#17) VHH antibodies, as disclosed in Tables 1, 2, 3, and 4, or framework regions substantially homologous thereto, and in particular framework regions that allow the maintenance of antigen specificity, for example framework regions that result in substantially the same or the same 3D structure of the antibody.

In certain preferred embodiments, all four of the variable heavy chain (SEQ ID NOs:5, 6, 7 and 8) framework regions (FR), as appropriate, or FR regions substantially homologous thereto, are found in the antibodies of the invention.

In other preferred embodiments, all four of the variable heavy chain (SEQ ID NOs:13, 14, 15 and 16) framework regions (FR), as appropriate, or FR regions substantially homologous thereto, are found in the antibodies of the invention.

In other preferred embodiments, all four of the variable heavy chain (SEQ ID NOs:21, 22, 23 and 24) framework regions (FR), as appropriate, or FR regions substantially homologous thereto, are found in the antibodies of the invention.

In other preferred embodiments, all four of the variable heavy chain (SEQ ID NOs:29, 30, 31 and 32) framework regions (FR), as appropriate, or FR regions substantially homologous thereto, are found in the antibodies of the invention.

In other preferred embodiments, all four of the variable heavy chain (SEQ ID NOs:37, 38, 39 and 40) framework regions (FR), as appropriate, or FR regions substantially homologous thereto, are found in the antibodies of the invention.

In other preferred embodiments, all four of the variable heavy chain (SEQ ID NOs:45, 46, 47 and 48) framework regions (FR), as appropriate, or FR regions substantially homologous thereto, are found in the antibodies of the invention.

In other preferred embodiments, all four of the variable heavy chain (SEQ ID NOs:53, 54, 55 and 56) framework regions (FR), as appropriate, or FR regions substantially homologous thereto, are found in the antibodies of the invention.

In other preferred embodiments, all four of the variable heavy chain (SEQ ID NOs:61, 62, 63 and 64) framework regions (FR), as appropriate, or FR regions substantially homologous thereto, are found in the antibodies of the invention.

In other preferred embodiments, all four of the variable heavy chain (SEQ ID NOs:69, 70, 71 and 72) framework regions (FR), as appropriate, or FR regions substantially homologous thereto, are found in the antibodies of the invention.

In certain preferred embodiments, in particular for the “type 2” antibodies of the invention, all four of the variable heavy chain (SEQ ID NOs:87, 88, 89 and 90) framework regions (FR), as appropriate, or FR regions substantially homologous thereto, are found in the antibodies of the invention.

In other preferred embodiments, all four of the variable heavy chain (SEQ ID NOs:95, 96, 97 and 98) framework regions (FR), as appropriate, or FR regions substantially homologous thereto, are found in the antibodies of the invention.

In other preferred embodiments, all four of the variable heavy chain (SEQ ID NOs:103, 104, 105 and 106) framework regions (FR), as appropriate, or FR regions substantially homologous thereto, are found in the antibodies of the invention.

In other preferred embodiments, all four of the variable heavy chain (SEQ ID NOs:111, 112, 113 and 114) framework regions (FR), as appropriate, or FR regions substantially homologous thereto, are found in the antibodies of the invention.

As described above, the present invention provides binding proteins, for example antibodies, which bind to (or specifically recognise or specifically bind to) CD163. CD163 is also known as M130, MM130, SCAR1, Macrophage-associated antigen, Hemoglobulin scavenger receptor, or Scavenger receptor cysteine rich Type 1 protein M130. Preferred binding proteins of the invention are antibodies, and in particular VHH antibodies. However, embodiments as described herein which relate to antibodies, e.g. VHH antibodies, apply equally, mutatis mutandis, to other types of binding proteins, or vice versa.

Preferred binding proteins are any single polypeptide chains which can bind (e.g. specifically bind) to porcine CD163. Appropriate types of binding protein which could be used in the invention are known in the art. For example, in some embodiments immunoglobulin based polypeptides are used, which generally comprise CDR regions (and optionally FR regions or an immunoglobulin based scaffold), such that the CDR regions (and optionally FR regions) of the antibodies of the invention can be grafted onto an appropriate scaffold or framework, e.g. an immunoglobulin scaffold.

However, in other embodiments, non-immunoglobulin based single chain binding proteins/scaffold proteins can be used which can be selected for the ability to specifically bind to a particular target antigen (CD163 or porcine CD163) in their own right. Such molecules are also referred to as antibody mimics (or antibody mimetics). Examples of appropriate non-immunoglobulin based single chain binding proteins are known and described in the art and include fibronectins (or fibronectin-based molecules), for example based on the tenth module of the fibronectin type III domain, such as Adnectins (e.g. from Compound Therapeutics, Inc., Waltham, MA); affimers (e.g. from Avacta); ankyrin repeat proteins or DARPins (e.g. from Molecular Partners AG, Zurich, Switzerland); lipocalins, e.g. anticalins (e.g. from Pieris Proteolab AG, Freising, Germany); human A-domains (e.g. Avimers); staphylococcal Protein A (e.g. from Affibody AG, Sweden); thioredoxins; and gamma-B-crystallin or ubiquitin based molecules, e.g. affilins (e.g. from Scil Proteins GmbH, Halle, Germany). Such molecules can also be used as scaffolds onto which appropriate CDRs which mediate target antigen binding can be grafted. For example, the CDR regions (and optionally FR regions) of the antibodies of the invention can be grafted onto an appropriate non-immunoglobulin scaffold.

In other embodiments of the invention, nucleic acid-based molecules such as aptamers can be used providing that such molecules can be selected for the ability to specifically bind to a particular target antigen (CD163 or porcine CD163) in their own right. Thus, where binding proteins are referred to herein, these embodiments can be extended to other types of binding entity or moiety such as nucleic acid-based molecules.

Preferred non-antibody binding proteins (or binding moieties) of the invention have the ability to bind to the same epitope as an anti-CD163 antibody of the invention and such binding proteins (or binding moieties) can for example be selected by way of competition assays such as those described elsewhere herein, using for example an antibody of the invention as a reference antibody.

CD163 is a 130 kDa type I transmembrane protein which has a signal peptide followed by nine scavenger-receptor cysteine rich (SRCR) domains, each approximately 100 amino acids in length, with a 35 amino-acid proline-serine-threonine (PST)-rich region separating SRCR domain 6 (SRCR6) and SRCR7. A second PST-rich region connects SRCR9 with the transmembrane domain and a short cytoplasmic tail, which contains a functional internalization motif. Surface expression of CD163 is restricted to cells of the monocyte-macrophage lineage.

Of particular relevance to the present invention, CD163 is expressed on the surface of porcine alveolar macrophages (PAMs), and is believed to play a vital role in the ability of various pathogens, including viral pathogens, notably PRRSV, to cause disease in pigs.

The binding proteins or antibodies of the present invention thus bind to or are capable of binding to CD163. In accordance with the present invention, the CD163 may be from any species, e.g. any mammalian species such as pig (porcine), human, cattle (bovine), dog (canine), cat (feline), sheep (ovine), horse (equine), mouse and monkey. In a preferred embodiment the CD163 is porcine CD163 and the antibodies bind to or are capable of binding to (or specifically recognise or specifically bind to) porcine CD163.

In certain embodiments the antibodies can cross-react with (or also bind to) other species of CD163. Thus, in some embodiments the antibodies can bind to porcine CD163, together with one or more other species, e.g. one or more other mammalian species, e.g. those mentioned above, of CD163. In some embodiments, the antibodies can bind to porcine CD163 and also to human CD163. In other words, the antibodies can cross-react with both porcine CD163 and human CD163. In other embodiments, the antibodies can bind to porcine CD163 but do not bind to (or do not significantly bind to or do not cross-react with) human CD163.

The binding proteins and antibodies of the invention can bind to any appropriate forms of CD163, in particular forms of CD163 which comprise the SRCR5 domain. Such forms can thus include full length CD163, or non-full length forms of CD163, for example truncated forms of CD163, or other variant forms of CD163 which for example contain a subset of SRCR domains, but generally include the SRCR5 domain. Preferred and convenient forms of CD163 to which the binding proteins and antibodies of the invention can bind include recombinant CD163, e.g. recombinant porcine CD163, or CD163 when expressed on the cell surface (cell-surface expressed CD163). Such cell-surface forms will thus in many cases represent a native or natural form of CD163, for example the form found on cells which naturally express or overexpress CD163.

Appropriate cell types which naturally express CD163 will be well known to a person skilled in the art and include monocytes and macrophages. A preferred cell type is PAMs. Alternatively, CD163 can be expressed or overexpressed, e.g. by recombinant means (or by other means of engineering) in a cell type which does not normally express CD163, in other words a cell expressing recombinant form of CD163 can be used.

Exemplary forms of CD163, e.g. recombinant CD163, as can be used herein in order to assess binding capability of the binding proteins and antibodies are full length CD163, or constructs containing subsets of different CD163 SRCR domains such as CD163-SRCR1-9, CD163-SRCR4-7 or CD163-SRCR5-6. Equally other combinations of CD163 SRCR domains and fragments containing subsets of different CD163 SRCR domains can be used providing all or part of (preferably all of) the SRCR5 domain is present. In some embodiments, the antibodies do not bind to (or do not significantly bind to) CD163 molecules which comprise a deletion of or within, or a mutation within, the SRCR5 domain. Porcine forms are preferably used to assess the antibodies of the present invention, although equivalent forms from other species, e.g. other mammalian species, may also be used, for example to assess for cross-reactivity.

The sequences of CD163 in various species are well known and described in the art and can be obtained for example from various sequence databases, e.g. Uniprot. For ease of reference, the porcine CD163 has the Uniprot number Q2VL90 and human CD163 has the Uniprot number Q86VB7.

Thus, preferred binding proteins or antibodies of the present invention have the ability to bind to the SRCR5 domain or an epitope in the SRCR5 domain, preferably the porcine SRCR5 domain, of CD163.

The sequence of the porcine SRCR5 domain is shown below and corresponds to residues 477-577 of Uniprot Q2VL90:

PRLVGGDIPCSGRVEVQHGDTWGTVCDSDFSLEAASVLCRELQCGTVVS
LLGGAHFGEGSGQIWAEEFQCEGHESHLSLCPVAPRPDGTCSHSRDVGVV
CS (SEQ ID NO:115).

The sequence of porcine CD163 is shown below and corresponds to the full sequence of Uniprot Q2VL90:

MDKLRMVLHENSGSADFRRCSAHLSSFTFAVVAVLSACLVTSSLGGKDKE
LRLTGGENKCSGRVEVKVQEEWGTVCNNGWDMDVVSVVCRQLGCPTAIKA
TGWANFSAGSGRIWMDHVSCRGNESALWDCKHDGWGKHNCTHQQDAGVTC
SDGSDLEMGLVNGGNRCLGRIEVKFQGRWGTVCDDNFNINHASVVCKQLE
CGSAVSFSGSANFGEGSGPIWFDDLVCNGNESALWNCKHEGWGKHNCDHA
EDAGVICLNGADLKLRVVDGVTECSGRLEVKFQGEWGTICDDGWDSDDAA
VACKQLGCPTAVTAIGRVNASEGTGHIWLDSVSCHGHESALWQCRHHEWG
KHYCNHDEDAGVTCSDGSDLELRLKGGGSHCAGTVEVEIQKLVGKVCDRS
WGLKEADVVCRQLGCGSALKTSYQVYSKTKATNTWLFVSSCNGNETSLWD
CKNWQWGGLSCDHYDEAKITCSAHRKPRLVGGDIPCSGRVEVQHGDTWGT
VCDSDFSLEAASVLCRELQCGTVVSLLGGAHFGEGSGQIWAEEFQCEGHE
SHLSLCPVAPRPDGTCSHSRDVGVVCSRYTQIRLVNGKTPCEGRVELNIL
GSWGSLCNSHWDMEDAHVLCQQLKCGVALSIPGGAPFGKGSEQVWRHMFH
CTGTEKHMGDCSVTALGASLCSSGQVASVICSGNQSQTLSPCNSSSSDPS
SSIISEENGVACIGSGQLRLVDGGGRCAGRVEVYHEGSWGTICDDSWDLN
DAHVVCKQLSCGWAINATGSAHFGEGTGPIWLDEINCNGKESHIWQCHSH
GWGRHNCRHKEDAGVICSEFMSLRLISENSRETCAGRLEVFYNGAWGSVG
RNSMSPATVGVVCRQLGCADRGDISPASSDKTVSRHMWVDNVQCPKGPDT
LWQCPSSPWKKRLASPSEETWITCANKIRLQEGNTNCSGRVEIWYGGSWG
TVCDDSWDLEDAQVVCRQLGCGSALEAGKEAAFGQGTGPIWLNEVKCKGN
ETSLWDCPARSWGHSDCGHKEDAAVTCSEIAKSRESLHATGRSSFVALAI
FGVILLACLIAFLIWTQKRRQRQRLSVFSGGENSVHQIQYREMNSCLKAD
ETDMLNPSGDHSEVQ (SEQ ID NO: 116).

Methods of assessing binding to (or ability to bind to) appropriate forms of CD163 would be well-known to a person skilled in the art and any appropriate method can be used.

A convenient and appropriate method for assessing binding include in vitro binding assays such as ELISA assays to assess binding of antibodies to immobilised antigen, such as immobilised forms of CD163 as described above. The skilled person will be familiar with ELISA assays and readily able to establish suitable conditions to assess the ability of a binding protein or antibody are to bind to CD163 in such an assay. A particularly preferred ELISA assay is described in the Examples section. Alternatively, or in addition, binding of antibodies to cell surface expressed CD163 can be assessed by any appropriate means, including by a flow cytometry assay (e.g. FACS analysis), for example using PAMs or cells expressing recombinant forms of CD163, e.g. forms as described elsewhere herein. A particularly preferred flow cytometry assay is described in the Examples section. Another method for testing for the ability of an antibody to bind to CD163 on the cell surface is immunohistochemistry.

In certain embodiments, binding proteins or antibodies of the present invention bind to CD163 (e.g. porcine CD163 or human CD163) in (as determined in) a Surface Plasmon Resonance (SPR) assay (e.g. a BIACore assay). Suitable SPR assays are known in the art. In certain preferred SPR assays, an appropriate form of CD163 is captured (or immobilised) on a solid support (e.g. a sensor chip), for example via amine coupling (e.g. 2000 Response Units (RU) CD163 is immobilized) and various concentrations (e.g. a dilution series, e.g. a doubling or trebling dilution series) of the binding proteins or antibodies to be tested is then injected. Preferred concentrations and flow-rates for injection are described in the Example section.

Such SPR assay methods can also conveniently be used to measure the binding kinetics of the antibody-antigen interaction, e.g. to determine association rate (ka), dissociation rate (kd) and affinity (KD). In a certain embodiments, measurements may be performed at 25°C in a suitable buffer, e.g. a standard HEPES-EDTA buffer such as HBS-EP (sold by GE Healthcare Life Sciences, 0.01 M HEPES pH 7.4, 0.15 M NaCl, 3mM EDTA, 0.0005% surfactant P20), at pH7.4. Kinetic parameters may be determined or calculated by any suitable model or software, for example by fitting the sensogram experimental data assuming a 1:1 interaction, for example using the BIAevaluation software. A particularly preferred SPR assay is described in the Examples section herein.

Thus, in a particularly preferred embodiment, binding proteins or antibodies of the present invention bind to CD163 (e.g. porcine or human CD163, preferably porcine CD163) in (as determined in, when assessed in) a Surface Plasmon Resonance (SPR) assay (e.g. a BIACore assay).

In certain preferred embodiments, antibodies of the present invention, when in VHH format, have a high binding affinity for CD163 (e.g. porcine CD163), e.g. have a K_{D} (equilibrium dissociation constant) in the range of 50 nM or lower (better).

Thus, preferably, antibodies of the invention, when in VHH format, have a binding affinity for CD163 (e.g. porcine CD163) that corresponds to a K_{D} of less than 100 nM, less than 80 nM, less than 60 nM, less than 50 nM, less than 45 nM, less than 40 nM, less than 35 nM, less than 30 nM, less than 25 nM, less than 20 nM, less than 15 nM or less than 10 nM, more preferably of less than 10.0, 9.5, 9.0, 8.5, 8.0, 7.5, 7.0, 6.5, 6.0, 5.5, 5.0, 4.5, 4.0, 3.5, 3.0, 2.5, 2.0, 1.5 or 1.0 nM. Particular exemplary binding affinities are disclosed in the Examples. Exemplary forms of CD163 that can be used to assess such binding affinity are recombinant porcine CD163 containing SRCR4-7 or recombinant porcine CD163 containing SRCR1-9. Appropriate exemplary forms are described in the Examples section, for example the constructs pCD163-SRCR4-7huFc or pCD163-SRCR1-9huFc. Thus, the binding affinities above may be observed when or if the antibodies of the invention are assayed using these constructs, e.g. in an SPR assay.

As mentioned above, in some embodiments of the invention, the antibodies can bind to porcine CD163 but do not bind to (or do not significantly bind to) human CD163. Viewed alternatively, they preferentially bind to porcine CD163 as opposed to human CD163.

As a preferred use of the binding proteins or antibodies of the invention is in the treatment or prevention of pathogenic infections which involve CD163, most notably PRRSV infection, typically, binding proteins or antibodies of the invention inhibit (or block or reduce) pathogen (e.g. PRRSV) infection, for example inhibit (or block or reduce) the ability of the pathogen, e.g. PRRSV, to cause infection (e.g. to infect appropriate host cells). Preferably, the inhibition or reduction is a measurable inhibition or reduction, more preferably a significant inhibition or reduction, e.g. a statistically significant inhibition or reduction such as with a probability value of ≤0.05 or <0.05. In certain embodiments, binding proteins or antibodies of invention can inhibit (or block or reduce) the ability of the pathogen, e.g. PRRSV, to infect host cells by at least 30%, at least 40%, at least 50%, at least 60%, at least 70%, at least 75%, at least 80%, at least 85%, at least 90%, at least 92%, at least 95% or at least 98%. Typically, such % inhibition (and other percentage inhibition levels as described herein) is in comparison with (or relative to) an appropriate control assay or control level, for example a control assay or control level in the absence of a binding protein or antibody (anti-CD163 antibody) (for example a negative control or background level or assay). Thus, a 0% inhibition (control) level (or conversely a 100% or maximum infection level) is typically the level in the absence of a binding protein or antibody (anti-CD163 antibody).

Such ability to inhibit infection can be determined or tested in any appropriate assay, examples of which would be readily derived by a person skilled in the art. Appropriate assays might for example be in vitro or ex vivo assays and for example involve the use of CD163 expressing host cells such as PAMs or recombinant CD163 expressing host cells as discussed elsewhere herein. Such cells can be brought into contact with PRRSV or other appropriate pathogens at a level which will cause infection of the cells. Appropriate assays may typically be carried out in the presence of serum, e.g. porcine serum or fetal bovine serum (FBS). The appropriate percentage of serum to use is readily determined by a skilled person, for example levels of 10% FBS and 80% porcine serum were used in the assays described in the Examples section. Ability of the binding proteins or antibodies of the invention to inhibit or reduce such infection can then readily be analysed, for example in comparison with (or relative to) a 100% infection level set by the control assay. An appropriate and exemplary infection assay is described in the Examples section.

Any appropriate concentrations of binding protein or antibody may be used to inhibit or reduce infection. Exemplary antibodies of the invention have the ability to cause inhibition, e.g. the levels of inhibition as outlined herein, with antibody, in particular VHH, when used at concentrations of at least 50, 60, 70, 80, 90, 100, 120, 140, 160, 180, 200, 300 or 400 µg/ml, e.g. at concentrations up to 200, 300 or 400 µg/ml, e.g. between 50 or 100 and 200, 300 or 400 µg/ml. If combinations of antibodies (e.g. VHH antibodies) are used then these levels in some embodiments can refer to the total amount of antibody (e.g. VHH) present, i.e. the sum of the individual concentrations of antibodies present.

In some embodiments, the binding protein or antibody of the invention can inhibit (or block or reduce) the ability of the PRRSV type 1 or the PRRSV type 2 to cause infection (e.g. to infect CD163 expressing host cells). In some embodiments, the binding protein or antibody of the invention can inhibit (or block or reduce) the ability of both type 1 PRRSV and type 2 PRRSV to cause infection (e.g. to infect CD163 expressing host cells). It can be noted that the binding protein or antibody of the invention targets host cell CD163 as opposed to the PRRSV (or other pathogenic entity) per se. This provides an important advantage of being able to inhibit infection by any virus, e.g. PRRSV, which uses the same binding region on CD163 for infection or pathogenesis. In this way, the antibodies, etc., of the invention can provide a means for blocking many strains or isolates of PRRSV, including high pathogenic strains or isolates, providing they use CD163 in order to infect cells. It is believed that CD163 utilisation is common for infection by multiple PRRSV strains. Thus, the antibodies of the present invention have wide utility. This is in contrast to for example some of the known approaches for PRRSV, e.g. vaccination, which can be strain specific, and their effectiveness (or whether they are effective at all) can vary depending on the strain. Thus, the antibodies of the invention provide important advantages and flexibility over such prior methods.

Preferred antibodies of the invention have the ability to almost completely inhibit type 1 PRRSV infection, for example at least 90% inhibition can be observed. Alternatively, at least 50%, 60%, 70%, 75% or 80% inhibition can be observed. In some embodiments antibodies which have the ability to show at least 80% inhibition of type 1 PRRSV infection, more preferably at least 85%, 90% or 95% inhibition are preferred.

Preferred antibodies of the invention have the ability to inhibit type 2 PRRSV infection by at least 50%, at least 55% or at least 60%, more preferably at least 65%, at least 70%, at least 75%, or at least 80% inhibition.

Some preferred antibodies of the invention have the ability to inhibit both type 1 and type 2 PRRSV infection, for example at the levels described above and elsewhere herein. Such antibodies are sometimes referred to herein as “dual” antibodies. Exemplary antibodies may therefore be capable of at least 50% inhibition of type 2 PRRSV, combined with at least 50%, 60%, 70%, 75%, 80%, 85%, 90% or 95% inhibition of type 1 PRRSV. Alternative exemplary antibodies may be capable of at least 55% or 60% inhibition of type 2 PRRSV, combined with at least 50%, 60%, 70%, 75%, 80%, 85%, 90% or 95% inhibition of type 1 PRRSV. Alternative exemplary antibodies may be capable of at least 65%, 70% or 75% inhibition of type 2 PRRSV, combined with at least 50%, 60%, 70%, 75%, 80%, 85%, 90% or 95% inhibition of type 1 PRRSV. In some embodiments, preferred antibodies of the invention may be capable of at least 65%, 70% or 75% inhibition of type 2 PRRSV, combined with at least 90% or 95% inhibition of type 1 PRRSV.

Exemplary “dual” antibodies in the form of VHH antibodies are 49(#18), 47(#19), 48(#20), 76(#2), 77(#16), 78(#8), 150(#15), 70(#23), and 144(#1), as shown in Tables A, B, C, D, E, F, G, H, and I, respectively.

In some embodiments, the binding protein or antibody of the invention can inhibit (or block or reduce) the ability of the PRRSV type 2 to infect host cells. In some embodiments, the binding protein or antibody of the invention has the ability to specifically inhibit (or block or reduce) the ability of the PRRSV type 2 to cause infection (e.g. to infect CD163 expressing host cells or specifically inhibit type 2 PRRSV infection). Such binding proteins or antibodies preferentially inhibit or reduce PRRSV type 2 infection as opposed to PRRSV type 1 infection. Exemplary antibodies may therefore be capable of at least 40%, 45% or 50% inhibition of type 2 PRRSV infection (e.g. inhibit the ability of type 2 PRRSV to infect host cells by at least 40%, 45% or 50%) .

In other embodiments such binding proteins or antibodies do not inhibit or reduce (e.g. do not significantly inhibit or reduce) type 1 PRRSV infection (e.g. do not inhibit or reduce, or do not significantly inhibit or reduce, the ability of type 1 PRRSV to infect host cells). Purely by way of example, such antibodies which do not significantly inhibit or reduce type 1 PRRSV infection may only reduce such infection by less than 10%, or less than 5%, or less than 2%, and preferably not at all (by 0%). Such antibodies are sometimes referred to herein as “type 2 specific” or “type 2 only” antibodies. Exemplary such antibodies in the form of VHH antibodies are 57(#11), 41(#12), 171(#14), and 29(#17), as shown in Tables 1, 2, 3 and 4, respectively.

The comparison between type 1 and type 2 PRRSV inhibition can readily be carried out using appropriate assays, for example in which the assay conditions are kept the same, e.g. using the same concentrations of test antibody or binding protein, but one assay is carried out with type 1 PRRSV and the other with type 2 PRRSV. Appropriate controls by which to assess such inhibition are also described elsewhere herein.

In certain embodiments, antibodies of the present invention have an IC₅₀ (e.g. for the inhibition of PRRSV1 infection of host cells, e.g. PAMs) of 350 µg/ml or less, 300 µg/ml or less, 280 µg/ml or less, 260 µg/ml or less, 240 µg/ml or less, 220 µg/ml or less, 200 µg/ml or less, 190 µg/ml or less, 180 µg/ml or less, 170 µg/ml or less, 160 µg/ml or less, 150 µg/ml or less, 140 µg/ml or less, 130 µg/ml or less, 120 µg/ml or less, 110 µg/ml or less, 100 µg/ml or less, 90 µg/ml or less, or 80 µg/ml or less. In some embodiments, the IC₅₀ is 80 to 350, 300, 250 or 200 µg/ml, or 80 to 160 µg/ml, or 80 to 120 µg/ml, or 100 to 200 µg/ml, or 100 to 160 µg/ml, or 100 to 120 µg/ml. Particular exemplary IC₅₀ values are also shown in the Examples.

In certain embodiments, antibodies of the present invention have an IC₅₀ (e.g. for the inhibition of PRRSV2 infection of host cells, e.g. PAMs) of 300 µg/ml or less, 280 µg/ml or less, 260 µg/ml or less, 240 µg/ml or less, 220 µg/ml or less, 210 µg/ml or less, 200 µg/ml or less, 180 µg/ml or less, 170 µg/ml or less, 160 µg/ml or less, 150 µg/ml or less, 140 µg/ml or less, 130 µg/ml or less, 120 µg/ml or less, 110 µg/ml or less or 100 µg/ml or less. In some embodiments, the IC₅₀ is 100 or 150 or 200 to 300 µg/ml, or 200 to 260 µg/ml, or 200 to 220 µg/ml, or 220 to 300 µg/ml, or 220 to 260 µg/ml, or 220 to 240 µg/ml. Particular exemplary IC₅₀ values are also shown in the Examples.

The preferred IC₅₀ values as described above are preferably as determined in an appropriate virus infectivity assay, e.g. as described above or in the Example section.

Although the dual antibodies as described herein show good ability to inhibit type 2 PRRSV infection, it is generally observed that the inhibition of type 2 PRRSV infection is not as complete or does not reach as high a level as the levels observed for the inhibition of type 1 PRRSV infection. Whilst not wishing to be bound by theory, it is possible that there is more than one epitope on CD163 which is involved in type 2 infection. Thus, in preferred embodiments of the invention the dual antibodies and the type 2 specific antibodies, e.g. as described herein, can be used in combination. Such combinations may be particularly useful when treatment or prevention of type 2 PRRSV infections is required or desired.

In alternative embodiments of the invention, the binding proteins or antibodies of the invention can be used to reduce the risk of or prevent PRRSV infection.

Preferably, the above described abilities and properties are observed at a measurable or significant level and more preferably at a statistically significant level, when compared to appropriate control levels. Appropriate significance levels are discussed elsewhere herein. More preferably, one or more of the above described abilities and properties are observed at a level which is measurably better, or more preferably significantly better (preferably statistically significantly better), when compared to the abilities observed for prior art antibodies.

In any statistical analysis referred to herein, preferably the statistically significant difference over a relevant control or other comparative entity or measurement has a probability value of ≤ 0.1 or < 0.1, preferably ≤ 0.05 or < 0.05. Appropriate methods of determining statistical significance are well known and documented in the art and any of these may be used.

In some embodiments, binding proteins or antibodies of the present invention have one or more, preferably two or more, or three or more, most preferably all, of the functional properties, in particular the preferred functional properties, described herein.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”.

In addition, where the terms “comprise”, “comprises”, “has” or “having”, or other equivalent terms are used herein, then in some more specific embodiments, for example in the definition of the CDR or FR sequences herein, these terms include the term “consists of” or “consists essentially of”, or other equivalent terms.

Nucleic acid molecules comprising nucleotide sequences that encode the binding proteins or antibodies of the present invention as defined herein or parts or fragments thereof, or nucleic acid molecules substantially homologous thereto, form yet further aspects of the invention.

Preferred nucleic acid molecules are those encoding a VHH antibody or a VH region or domain of the present invention (e.g., those encoding SEQ ID NO:1, 9, 17, 25, 33, 41, 49, 57 or 65). Other preferred nucleic acid molecules are those encoding the sets of three CDR sequences as defined in any one of Tables A, B, C, D, E, F, G, H or I. Preferred such nucleic acid molecules also encode appropriate framework regions, e.g. FR1, FR2, FR3 and FR4 regions, preferably the sets of FR sequences as defined in any one of Tables A, B, C, D, E, F, G, H or I.

In other embodiments, preferred nucleic acid molecules are those encoding a VHH antibody or a VH region or domain of the present invention (e.g., those encoding SEQ ID NO:83, 91, 99 or 107). Other preferred nucleic acid molecules are those encoding the sets of three CDR sequences as defined in any one of Tables 1, 2, 3 or 4. Preferred such nucleic acid molecules also encode appropriate framework regions, e.g. FR1, FR2, FR3 and FR4 regions, preferably the sets of FR sequences as defined in any one of Tables 1, 2, 3 or 4 (e.g. the type 2 specific antibodies of the invention).

The term “substantially homologous” as used herein in connection with an amino acid or nucleic acid sequence includes sequences having at least 60%, 65%, 70% or 75%, preferably at least 80%, and even more preferably at least 85%, 90%, 95%, 96%, 97%, 98% or 99%, sequence identity to the amino acid or nucleic acid sequence disclosed. Substantially homologous sequences of the invention thus include single or multiple base or amino acid alterations (additions, substitutions, insertions or deletions) to the sequences of the invention. At the amino acid level preferred substantially homologous sequences contain up to 5, e.g. only 1, 2, 3, 4 or 5, preferably 1, 2, 3 or 4, preferably 1, 2 or 3, more preferably 1 or 2, altered amino acids, in one or more of the framework regions and/or one or more of the CDRs making up the sequences of the invention. Said alterations can be with conservative or non-conservative amino acids. Preferably said alterations are substitutions, preferably conservative amino acid substitutions.

In certain embodiments, if a given starting sequence is relatively short (e.g. five amino acids in length), then fewer amino acid substitutions may be present in sequences substantially homologous thereto as compared with the number of amino acid substitutions that might optionally be made in a sequence substantially homologous to a longer starting sequence. For example, in certain embodiments, a sequence substantially homologous to a starting VH CDR1 sequence in accordance with the present invention, e.g. a starting VH CDR1 sequence which in some embodiments may be five amino acid residues in length, preferably has 1 or 2 (more preferably 1) altered amino acids in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the CDRs, for example a sequence identity of at least 60%, 70%, 75%, 80%, 85%, 90%, 95%, 96%, 97%, 98% or 99%.

Routine methods in the art such as alanine scanning mutagenesis and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

Once identified, the addition, deletion, substitution or insertion of one or more amino acids in the amino acid sequence of a parent antibody to form a new antibody, wherein said parent antibody is one of the antibodies of the invention as defined elsewhere herein, and testing the resulting new antibody to identify antibodies that bind to CD163 in accordance with the invention can be carried out using techniques which are routine in the art. Such methods can be used to form multiple new antibodies that can all be tested for their ability to bind CD163. Preferably said addition, deletion, substitution or insertion of one or more amino acids takes place in one or more of the CDR domains.

For example, said manipulations could conveniently be carried out by genetic engineering at the nucleic acid level wherein nucleic acid molecules encoding appropriate binding proteins and domains thereof are modified such that the amino acid sequence of the resulting expressed protein is in turn modified in the appropriate way. Testing the ability of one or more of the modified antibodies to bind to CD163 can be carried out by any appropriate method, which are well known and described in the art. Suitable methods are also described elsewhere herein and in the Examples section.

New antibodies produced, obtained or obtainable by these methods form a yet further aspect of the invention.

The term “substantially homologous” also includes modifications or chemical equivalents of the amino acid and nucleotide sequences of the present invention that perform substantially the same function as the proteins or nucleic acid molecules of the invention in substantially the same way. For example, any substantially homologous antibody should retain the ability to bind to CD163 as described above. Preferably, any substantially homologous antibody should retain one or more (or all) of the functional capabilities of the starting antibody.

Preferably, any substantially homologous antibody should retain the ability to specifically bind to the same epitope of CD163 as recognized by the starting antibody in question, for example, the same epitope recognized by the CDR domains of one or more of the antibodies of the invention or the VH (VHH) domains of the invention as described herein, e.g. bind to the same epitope as one or more of the various antibodies of the invention (e.g. one or more of the VHH antibodies 49(#18), 47(#19), 48(#20), 76(#2), 77(#16), 78(#8), 150(#15), 70(#23), or 144(#1), as shown in Tables A, B, C, D, E, F, G, H, and I, respectively). Thus, preferably, any substantially homologous antibody should retain the ability to compete, in a suitable assay, with one or more of the various antibodies of the invention (e.g. VHH antibodies 49(#18), 47(#19), 48(#20), 76(#2), 77(#16), 78(#8), 150(#15), 70(#23), or 144(#1), as shown in Tables A, B, C, D, E, F, G, H, and I, respectively) for binding to CD163.

In other embodiments, any substantially homologous antibody should retain the ability to specifically bind to the same epitope of CD163 as recognized by the starting antibody in question, for example, the same epitope recognized by the CDR domains of one or more antibodies of the invention or the VH (VHH) domains of the invention as described herein, e.g. bind to the same epitope as one or more of the various type 2 antibodies of the invention (e.g. one or more of the VHH antibodies 57(#11), 41 (#12), 171 (#14), or 29(#17), as shown in Tables 1, 2, 3 and 4, respectively). Thus, preferably, any substantially homologous antibody should retain the ability to compete with one or more of the various type 2 antibodies of the invention (e.g. VHH antibodies 57(#11), 41(#12), 171(#14), or 29(#17), as shown in Tables 1, 2, 3 and 4, respectively, e.g. the type 2 specific antibodies of the invention) for binding to CD163.

Binding to the same epitope/antigen can be readily tested by methods well known and described in the art, e.g. using binding assays, e.g. a competition assay or by analysis of the crystal structure of the antigen-antibody complex. Retention of other functional properties can also readily be tested by methods well known and described in the art or herein.

Thus, a person skilled in the art will appreciate that binding assays can be used to test whether any antibodies, for example “substantially homologous” antibodies, have the same binding specificities, e.g. bind to the same epitope, or with the same or equivalent affinity, as the antibodies and antibody fragments of the invention, for example, binding assays such as competition assays or ELISA assays as described elsewhere herein. BIAcore assays could also readily be used to establish whether antibodies, for example “substantially homologous” antibodies, can bind to CD163. The skilled person will be aware of other suitable methods and variations.

As outlined below, a competition binding assay can be used to test whether antibodies, for example “substantially homologous” antibodies retain the ability to specifically bind to substantially the same epitope of CD163 as recognized by one or more of the antibodies of the invention as shown in the various sequence Tables herein, or have the ability to compete with one or more of the various antibodies of the invention as shown in the various sequence Tables herein. The method described below is only one example of a suitable competition assay. The skilled person will be aware of other suitable methods and variations.

An exemplary competition assay involves assessing the binding of various effective concentrations of an antibody of the invention to CD163 in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to CD163) is evidence of binding to substantially the same epitope. Preferably, the test antibody significantly reduces the amount of antibody of the invention that binds to CD163. Preferably, the test antibody reduces the amount of antibody of the invention that binds to CD163 by at least about 95%. ELISA and flow cytometry assays may be used for assessing inhibition of binding in such a competition assay but other suitable techniques would be well known to a person skilled in the art.

Such antibodies (monoclonal antibodies) which have the ability to specifically bind to substantially the same (or the same) epitope of CD163 or an overlapping epitope of CD163 as recognized by the antibodies of the invention (e.g. VHH antibodies 49(#18), 47(#19), 48(#20), 76(#2), 77(#16), 78(#8), 150(#15), 70(#23), or 144(#1), as shown in Tables A, B, C, D, E, F, G, H, and I, respectively) or which have the ability to compete with one or more of the various antibodies of the invention (e.g. VHH antibodies 49(#18), 47(#19), 48(#20), 76(#2), 77(#16), 78(#8), 150(#15), 70(#23), or 144(#1), as shown in Tables A, B, C, D, E, F, G, H, and I, respectively) are further embodiments of the present invention.

In another embodiment, antibodies (monoclonal antibodies) which have the ability to specifically bind to substantially the same (or the same) epitope of CD163 or an overlapping epitope of CD163 as recognized by the antibodies of the invention (e.g. VHH antibodies 57(#11), 41(#12), 171(#14), or 29(#17), as shown in Tables 1, 2, 3 and 4, respectively) or which have the ability to compete with one or more of the various antibodies of the invention (e.g. VHH antibodies 57(#11), 41(#12), 171(#14), or 29(#17), as shown in Tables 1, 2, 3 and 4, respectively, e.g. the type 2 specific antibodies of the invention) are further embodiments of the present invention. In some embodiments, a preferred such antibody is the VHH antibody 171(#14) comprising SEQ ID NO: 99 (or the relevant three CDR sequences of said sequence) as outlined in Table 3.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to CD163. Preferably, the competing antibody can bind to the same epitope as the reference antibody. Alternatively viewed, the competing antibody preferably has the same epitope specificity as the reference antibody.

“Reference antibodies” as used herein are antibodies which can bind to CD163 in accordance with the invention which preferably have a VH domain as defined herein, more preferably have a VH domain or are a VHH antibody comprising SEQ ID NO: 1, 9, 17, 25, 33, 41, 49, 57, or 65 (or the relevant three CDR sequences of said sequences) as outlined in Tables A, B, C, D, E, F, G, H, or I.

Other “Reference antibodies” as used herein are antibodies which can bind to CD163 in accordance with the invention which preferably have a VH domain as defined herein, more preferably have a VH domain or are a VHH antibody comprising SEQ ID NO: 83, 91, 99 or 107 (or the relevant three CDR sequences of said sequences) as outlined in Tables 1, 2, 3 or 4 (e.g. the type 2 specific antibodies of the invention). In some embodiments, a preferred reference antibody is a VHH antibody comprising SEQ ID NO: 99 (or the relevant three CDR sequences of said sequence) as outlined in Table 3.

The identification of one or more competing antibodies or antibodies that bind to the same epitope is a straightforward technical matter now that reference antibodies such as those outlined in the sequence Tables herein have been provided. As the identification of competing antibodies or antibodies that bind to the same epitope can be determined in comparison to a reference antibody, it will be understood that actually determining the epitope to which either or both antibodies bind is not in any way required in order to identify a competing antibody or an antibody that binds to the same epitope. However, epitope mapping can be performed using standard techniques, if desired.

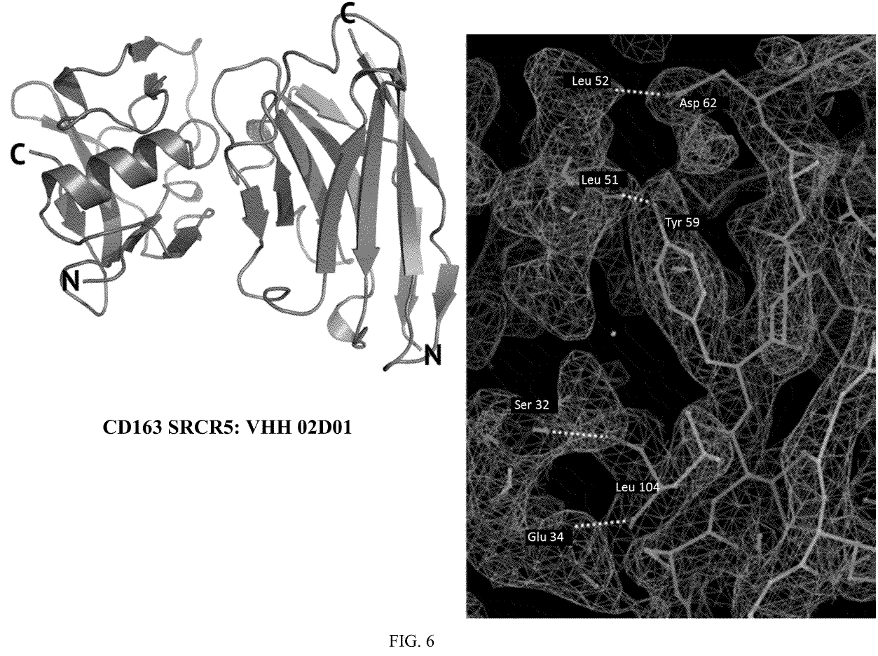

Analysis of the crystal structure of the antigen-antibody complex between the SRCR5 domain of porcine CD163 as set out in SEQ ID NO:115 and the VHH antibody 171(#14), i.e. VHH 014 (2D01), which has the amino acid sequence (SEQ ID NO: 17′ or 99) as shown in Table 3, has been carried out to determine the region (epitope) in CD163 to which this antibody binds (see FIG. 6). The residues on porcine CD163 which contribute to antigen binding have been identified as S507, E509, L526 and L527 of porcine CD163 (with reference to the Uniprot Q2VL90 sequence as set out in SEQ ID NO:116). The crystal structure shows that S507 and E509 interact with L104 of the VHH 014 (2D01) and L526 interacts with Y59 of the VHH 014 (2D01) and L527 interacts with D62 of the VHH 014 (2D01).

### Epitope on CD163

Thus, a further aspect provides an antibody (or binding protein) comprising an antigen binding domain which binds or specifically binds to porcine CD163, wherein said antibody (antigen binding domain) binds to an epitope in the SRCR5 domain of porcine CD163 comprising (or defined by) amino acids S507, E509, L526 and L527 of SEQ ID NO:116, or corresponding residues in an alternative CD163 sequence, e.g. a CD163 sequence from another species.

Viewed alternatively, a further aspect provides an antibody (or binding protein) comprising an antigen binding domain which binds or specifically binds to porcine CD163, wherein said antibody (antigen binding domain) binds to an epitope in the SRCR5 domain of porcine CD163 comprising (or defined by) amino acids S32, E34, L51 and L52 of SEQ ID NO:115, or corresponding residues in an alternative CD163 sequence, e.g. a CD163 sequence from another species. The relevant residues are shown underlined in SEQ ID NO:115 below.

PRLVGGDIPCSGRVEVQHGDTWGTVCDSDFSLEAASVLCRELQCGTVVSL LGGAHFGEGSGQIWAEEFQCEGHESHLSLCPVAPRPDGTCSHSRDVGVVC
S (SEQ ID NO: 115).

In particular, the interaction between the CDR2 of the VHH and the residues L526 and L527 of porcine CD163 (SEQ ID NO:116) look to be important for the antigen-antibody (antigen binding domain) interaction. Thus, a further aspect of the invention provides an antibody (or binding protein) comprising an antigen binding domain which binds or specifically binds to porcine CD163, wherein said antibody (antigen binding domain) binds to an epitope in the SRCR5 domain of porcine CD163 comprising (or defined by) amino acids L526 and L527 of SEQ ID NO: 116, or corresponding residues in an alternative CD163 sequence, e.g. a CD163 sequence from another species.

In other embodiments, the invention provides an antibody (or binding protein) comprising an antigen binding domain which binds or specifically binds to porcine CD163, wherein said antibody (antigen binding domain) binds to an epitope in the SRCR5 domain of porcine CD163 comprising (or defined by) amino acids L526, L527 and S507, or L526, L527 and E509, or amino acids L526, L527, S507 and E509 of SEQ ID NO:116, or corresponding residues in an alternative CD163 sequence, e.g. a CD163 sequence from another species.

In other embodiments said antibody (or binding protein) binds to an epitope in the SRCR5 domain of porcine CD163 comprising (or defined by) one, two, three or all of the residues S507, E509, L526 and L527 of SEQ ID NO:116, or corresponding residues in an alternative CD163 sequence, e.g. a CD163 sequence from another species. In other words, at least one amino acid of the epitope on CD163 bound by the antibody (or binding protein) of the invention comprises S507, E509, L526 or L527 of SEQ ID NO: 116, or corresponding residues in an alternative CD163 sequence, e.g. a CD163 sequence from another species. Such antibodies can be regarded as examples of antibodies which bind to overlapping epitopes.

Viewed alternatively, said antibody (or binding protein) binds to an epitope in the SRCR5 domain of porcine CD163 comprising (or defined by) one, two, three or all of the residues S32, E34, L51 and L52 of SEQ ID NO:115, or corresponding residues in an alternative CD163 sequence, e.g. a CD163 sequence from another species. In other words, at least one amino acid of the epitope on CD163 bound by the antibody (or binding protein) of the invention comprises S32, E34, L51 or L52 of SEQ ID NO:115, or corresponding residues in an alternative CD163 sequence, e.g. a CD163 sequence from another species. Such antibodies can be regarded as examples of antibodies which bind to overlapping epitopes.

To the inventors’ knowledge, monoclonal antibodies which can bind or specifically bind to porcine CD163, in particular to an epitope in the SRCR5 domain of porcine CD163, and which can inhibit or reduce Type 2 PRRSV infection have not been described in the art, either in a form where only Type 2 PRRSV infection is inhibited or reduced, or in a form where said antibodies are capable of inhibiting or reducing Type 1 and Type 2 PRRSV infection.

Thus, the individual monoclonal antibodies as described herein are both unusual and advantageous. In addition, as set out above, the inventors’ believe they have identified an epitope on porcine CD163 which is important for Type 2 PRRSV infection and which is therefore a target for antibodies and binding proteins in general in order to reduce or inhibit PRRSV infection. It can further be noted that the residues on the porcine CD163 identified herein as being part of the epitope, are located in different regions of CD163 than those previously identified as potentially being important for PRRSV infection. For example, previous reports, e.g. Ma et al., 2017 (Am. Soc. For Microbiology, 91(3):e01897-16), identified the residue R561 in the SRCR5 domain of CD163 as being important for Type 1 PRRSV infection. This residue is found in loop 5-6 of porcine CD163, which is located between residues Phe 544 and Arg 570 of CD163. Other reports have speculated that the ligand-binding pocket (LBP) in CD163, which is located between residues S487 and G499 of CD163, might also be an important region for PRRSV infection. None of the four residues identified as being part of the epitope in the current study are in these regions.

Thus it is believed that the present invention has identified a novel epitope in a distinct part of the SRCR5 region of porcine CD163 that is important for PRRSV infection, in particular type 2 PRRSV infection, and antibodies (or binding proteins) that bind to this epitope or an overlapping epitope are particularly preferred. As set out above, the antibody 171 (#14) shown in Table 3 has been shown to bind to this epitope. Initial experiments using competition binding studies show that at least the antibodies 57(#11), 70(#23), 144(#1) and 150(#15) may bind to the same or an overlapping epitope.

### Paratope on Antibody

The two L residues, L526 and L527, in CD163 have been shown to interact with Y59 and D62 in a YYAD motif found in the CDR2 of the VHH antibody 171(#14), i.e. VHH 014 (2D01). This VHH antibody has been shown to have an inhibitory effect on or to reduce Type 2 PRRSV infection. It can be noted that the sequence YYAD or a sequence highly similar to the sequence YYAD is found in the equivalent or corresponding region of the CDR2 of all the VHH antibodies described herein. All the VHH antibodies described herein have been shown to have an inhibitory effect on or to reduce Type 2 PRRSV infection. Thus, this VH CDR2 region seems to be an important feature in antibodies (e.g. VHH antibodies) that have the ability to inhibit or reduce Type 2 PRRSV infection.

Thus, preferred antibodies (or binding proteins) of the invention comprise a CDR2, in particular a VH CDR2, which comprises the amino acid sequence YAD or YAE, preferably XYAD or XYAE, in which X can be any amino acid, preferably Y, L, P, N, F, or R, more preferably Y, F, L, N or R, or Y, P or L, most preferably Y. In other embodiments, the sequence can comprise YAN or XYAN as an alternative to YAD or YAE.

In embodiments the X residue is located at position 59 in SEQ ID NO:17′ or of the VHH antibody 171(#14), i.e. VHH 014 (2001) as shown in Table , or the corresponding position in the VH CDR2 of an alternative antibody (or VHH). Viewed alternatively, the X residue is located at position 10 in SEQ ID NO:19′ or 101 (CDR2) of the VHH antibody 171 (#14), i.e. VHH 014 (2D01) as shown in Table 3, or the corresponding position in the VH CDR2 of an alternative antibody (or VHH), which can for example be at position 8 or 9 in the CDR2 regions of other VHH antibodies as described herein. The positions of the other residues in the XYAD or XYAE or XYAN motifs can be determined accordingly with reference to these positions.

The two residues, S507 and E509, in CD163 have been shown to interact with L104 in the CDR3 of the VHH antibody 171(#14), i.e. VHH 014 (2D01). This VHH antibody has been shown to have an inhibitory effect on or to reduce Type 2 PRRSV infection. Thus, this VH CDR3 residue (or the corresponding residue in other antibodies, e.g. VHH antibodies) may be an important residue in antibodies (e.g. VHH antibodies) that have the ability to inhibit or reduce Type 2 PRRSV infection.

Thus, in some embodiments, antibodies (or binding proteins) of the invention comprise a CDR3, in particular a VH CDR3 which comprises the amino acid residue L at position 104 in SEQ ID NO:17′ or 99 of the VHH antibody 171(#14), i.e. VHH 014 (2D01) as shown in Table 3, or the corresponding position in the VH CDR3 of an alternative antibody (or VHH). Viewed alternatively, the L residue is located at position 6 in SEQ ID NO:2′ or 102 of the VHH antibody 171(#14), i.e. VHH 014 (2D01) as shown in Table 3, or the corresponding position in the VH CDR3 of an alternative antibody (or VHH).

In some embodiments, the above L residue in CDR3 is present in addition to the above described YAD or YAE or YAN sequence in CDR2, preferably XYAD or XYAE or XYAN, in which X can be any amino acid, preferably Y, L, P, N, F, or R, more preferably Y, F, L, N or R, or Y, P or L, most preferably Y.

In embodiments of the invention where substantially homologous sequences are provided, in some embodiments the residues YAD, YAE or YAN, or XYAD, XYAE or XYAN, as defined above are maintained or present, and the variation occurs outside these residues.

Substantially homologous sequences of proteins of the invention include, without limitation, conservative amino acid substitutions, or for example alterations that do not affect the VH, VL or CDR domains of the antibodies, e.g. antibodies where tag sequences, toxins or other components are added that do not contribute to the binding of antigen, or alterations to convert one type or format of binding protein, antibody molecule or fragment to another type or format of binding protein, antibody molecule or fragment (e.g. conversion from VHH to Fab or scFv or whole antibody or vice versa), or the conversion of an antibody molecule to a particular class or subclass of antibody molecule (e.g. the conversion of an antibody molecule to IgG or a subclass thereof, e.g. IgG₂).

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). In other examples, families of amino acid residues can be grouped based on hydrophobic side groups or hydrophilic side groups.

Homology may be assessed by any convenient method. However, for determining the degree of homology between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having at least 60%, 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

The terms “antibody” and “immunoglobulin”, as used herein, refer broadly to any immunological binding agent that comprises an antigen binding domain, including polyclonal and monoclonal antibodies. Monoclonal antibodies are however preferred. In other words, in some embodiments antibodies of the invention are not polyclonal antibodies. Depending on the type of constant domain in the heavy chains, whole antibodies are assigned to one of five major classes: IgA, IgD, IgE, IgG, and IgM and the antibodies of the invention may be in any one of these classes. Several of these are further divided into subclasses or isotypes, such as IgG1, IgG2, IgG3, IgG4, and the like. The heavy-chain constant domains that correspond to the difference classes of immunoglobulins are termed α, δ, ε, γ and µ, respectively. The subunit structures and three-dimensional configurations of different classes of immunoglobulins are well known.

Generally, where whole antibodies rather than antigen binding regions are used in the invention, IgG are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains.

The term “heavy chain complementarity determining region” (“heavy chain CDR”) as used herein refers to regions of hypervariability within the heavy chain variable region (V_{H} domain) of an antibody molecule or within a VHH antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “heavy chain variable region” (V_{H} domain) as used herein refers to the variable region of a heavy chain of an antibody molecule.

The term “light chain complementarity determining region” (“light chain CDR”) as used herein refers to regions of hypervariability within the light chain variable region (V_{L} domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

The term “light chain variable region” (V_{L} domain) as used herein refers to the variable region of a light chain of an antibody molecule.

As will be understood by those in the art, the immunological binding reagents encompassed by the term “antibody” includes or extends to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like.

The techniques for preparing and using various antibody-based constructs and fragments are well known in the art.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

In all embodiments of the invention, single domain antibodies (also referred to as VHH antibodies, sdAbs, DABs, dAbs, nanobodies, camelid antibodies, vNAR (shark) antibodies, VH antibodies or VL antibodies) are preferred, in particular VHH antibodies, nanobodies, camelid antibodies, and vNAR (shark) antibodies. Such antibodies comprise a single monomeric variable antibody domain, usually a VH domain, which can bind to antigen (although single VL domains which have the ability to bind antigen have been described and can be used). Thus, in some such preferred embodiments the antibodies (or antigen binding domains) of the invention comprise one (or a single) heavy chain variable region (VH or VHH), although in some embodiments a number of these individual heavy chain variable regions with the same or different sequences can be present together in the same construct or molecule.

Such antibodies can be obtained or prepared using standard techniques which are well known and described in the art. For example, such antibodies can be obtained by immunizing appropriate animals, e.g. camelids such llamas, or sharks, with the desired antigen and then cloning the VH domains of the antibodies generated into appropriate expression vectors and selecting for binders. Libraries of VH domains (e.g. phage display libraries of human VH domains) are also available or can be generated and can then be screened.

Due to their relatively small size, single domain antibodies can have a relatively short half life, e.g. a relatively short plasma half life. Thus, such antibodies are sometimes modified in order to extend or prolong their half life. Techniques to do this are well known and described in the art and any of these may be used. Examples include attaching or conjugating or fusing the antibodies to albumin (or another protein or entity which itself has a long (or longer) half life), or attaching or conjugating or fusing the antibodies to another protein or entity which can itself interact with a protein or entity which has a long (or longer) half life, or attaching or conjugating the antibodies to PEG (or other polymers), or attaching or conjugating or fusing the antibodies to an antibody, or other protein or entity, which binds to FcRn.

In certain embodiments, the antibody or antibody fragment of the present invention comprises all or a portion of a heavy chain constant region, such as an IgG1, IgG2, IgG3, IgG4, IgA1, IgA2, IgE, IgM or IgD constant region. Preferably, the heavy chain constant region is an IgG heavy chain constant region, e.g. an IgG2 heavy chain constant region, or a portion thereof. Furthermore, the antibody or antibody fragment can comprise all or a portion of a kappa light chain constant region or a lambda light chain constant region, or a portion thereof. All or part of such constant regions may be produced naturally or may be wholly or partially synthetic. Appropriate sequences for such constant regions are well known and documented in the art. When a full complement of constant regions from the heavy and light chains are included in the antibodies of the invention, such antibodies are typically referred to herein as “full length” antibodies or “whole” antibodies. In some embodiments, IgG₂ antibodies are preferred.

In other embodiments it is preferred that no constant regions, e.g. no heavy chain or light chain constant regions, are present, e.g. a variable domain or heavy chain variable domain (VH) is the only part of an antibody that is present.

The antibodies or antibody fragments can be produced naturally or can be wholly or partially synthetically produced.

Many antibodies or antibody fragments comprise an antibody light chain variable region (V_{L}) that comprises three CDR domains and an antibody heavy chain variable region (V_{H}) that comprises three CDR domains. Said VL and VH generally form the antigen binding site.

However, it is well documented in the art that the presence of three CDRs from the light chain variable domain and three CDRs from the heavy chain variable domain of an antibody is not always necessary for antigen binding. Thus, constructs smaller than the above classical antibody fragment are known to be effective.

For example, camelid antibodies have an extensive antigen binding repertoire but are devoid of light chains. Also, results with single domain antibodies comprising VH domains alone or VL domains alone show that these domains can bind to antigen with acceptably high affinities and have other advantages such as their small size and ease of production. Thus, three CDRs can effectively bind antigen and such single domain antibodies (for example VHH antibodies, sdAbs, DABs, dAbs, nanobodies, camelid antibodies, vNAR (shark) antibodies, VH antibodies or VL antibodies, in particular VHH antibodies, nanobodies, camelid antibodies, and vNAR (shark) antibodies) are exemplified herein and this type of antibody is preferred (e.g. a VHH antibody).

The antibody, binding protein and nucleic acid molecules of the invention are generally “isolated” or “purified” molecules insofar as they are distinguished from any such components that may be present in situ within a human or animal body (e.g. a camelid) or a tissue sample derived from a human or animal body (e.g. a camelid). The sequences may, however, correspond to or be substantially homologous to sequences as found in a human or animal body (e.g. a camelid). Thus, the term “isolated” or “purified” as used herein in reference to nucleic acid molecules or sequences and proteins or polypeptides, e.g. antibodies, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

It can be noted that the antibodies etc., of the invention do not occur in nature and are, in that respect, man-made constructs in that they do not correspond to molecules that occur naturally. For example, preferred antibodies are single domain antibodies which can be engineered or recombinantly produced, and even in species that produce such antibodies naturally, e.g. camelids, such species will not produce antibodies to CD163, in particular porcine CD163, unless they are experimentally induced to do so, e.g. by immunization. In other words the antibodies, etc., of the invention are non-native.

The term “fragment” as used herein refers to fragments of biological relevance, e.g. fragments that contribute to antigen binding, e.g. form part of the antigen binding site, and/or contribute to the functional properties of the CD163 antibody. Certain preferred fragments comprise or consist of a heavy chain variable region (V_{H} domain or the three VH CDRs) of the antibodies of the invention.

A person skilled in the art will appreciate that the proteins and polypeptides of the invention, such as the heavy and light chain CDRs, the heavy and light chain variable regions, antibodies and antibody fragments, may be prepared in any of several ways well known and described in the art, but are most preferably prepared using recombinant methods.

Nucleic acid fragments encoding the heavy and light chain variable regions of the antibodies of the invention, as appropriate, can be derived or produced by any appropriate method, e.g. by cloning or synthesis.

Once nucleic acid fragments encoding the heavy and/or light chain variable regions of the antibodies of the invention have been obtained, these fragments can be further manipulated by standard recombinant DNA techniques, for example to convert the variable region fragments into full length antibody molecules with appropriate constant region domains, or into particular formats of antibody fragment discussed elsewhere herein, e.g. single domain antibodies such as VHH, Fab fragments, scFv fragments, etc. Typically, or as part of this further manipulation procedure, the nucleic acid fragments encoding the antibody molecules of the invention are generally incorporated into one or more appropriate expression vectors in order to facilitate production of the antibodies of the invention or for example to facilitate selection or screening, e.g. by incorporating into phage display vectors.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates an expression vector, e.g. a recombinant expression vector containing or comprising a nucleic acid molecule of the invention, or a fragment thereof, and the necessary regulatory sequences for the transcription and translation of the protein sequence encoded by the nucleic acid molecule of the invention.

Expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, NY, 1989) and other laboratory textbooks.

Suitable host cells include a wide variety of eukaryotic host cells and prokaryotic cells. For example, the proteins of the invention may be expressed in yeast cells or mammalian cells. In addition, the proteins of the invention may be expressed in prokaryotic cells, such as Escherichia coli.

The proteins of the invention may also be prepared by chemical synthesis using techniques well known in the chemistry of proteins such as solid phase synthesis.

A yet further aspect provides an expression construct or expression vector or expression system (e.g. a viral or bacterial or other expression construct, vector or system) comprising one or more of the nucleic acid fragments or segments or molecules of the invention. Preferably the expression constructs or vectors or systems are recombinant. Preferably said constructs or vectors or systems further comprise the necessary regulatory sequences for the transcription and translation of the protein sequence encoded by the nucleic acid molecule of the invention. Preferred constructs etc., are those which allow prolonged or sustained expression of the antibodies (or binding proteins) of the invention within the host target species, e.g. within pigs. Such expression can be transient, e.g. episomal, or more permanent, e.g. via genomic integration, providing sufficient levels and length of expression are achieved in order for a therapeutic or biological effect to be observed.

A yet further aspect provides a host cell (e.g. a mammalian or bacterial or yeast host cell) or virus comprising one or more expression constructs or expression vectors of the invention. Also provided are host cells or viruses comprising one or more of the nucleic acid molecules of the invention. A host cell (e.g. a mammalian host cell or bacterial host cell, or yeast host cell) or virus expressing an antibody (or binding protein) of the invention forms a yet further aspect.

Such expression constructs or vectors or systems, or host cells or viruses, or other nucleic acid products or fragments encoding the antibodies (or binding proteins) of the invention can be administered as therapeutic agents to a subject to allow the production of the antibodies (or binding proteins) of the invention in situ within the subject and thereby exert their therapeutic effects.

A yet further aspect of the invention provides a method of producing (or manufacturing) an antibody of the present invention comprising a step of culturing the host cells of the invention. Preferred methods comprise the steps of (i) culturing a host cell comprising one or more of the recombinant expression vectors or one or more of the nucleic acid sequences of the invention under conditions suitable for the expression of the encoded antibody or protein; and optionally (ii) isolating or obtaining the antibody or protein from the host cell or from the growth medium/supernatant. Such methods of production (or manufacture) may also comprise a step of purification of the antibody or protein product and/or formulating the antibody or product into a composition including at least one additional component, such as a pharmaceutically acceptable carrier or excipient.

In embodiments when the antibody or protein of the invention is made up of more than one polypeptide chain (e.g. certain fragments such as Fab fragments or whole antibodies), then all the polypeptides are preferably expressed in the host cell, either from the same or a different expression vector, so that the complete proteins, e.g. antibody proteins of the invention, can assemble in the host cell and be isolated or purified therefrom.

In another aspect, the invention provides a method of binding CD163, comprising contacting a composition comprising CD163 with an antibody of the invention.

In yet another aspect, the invention provides a method of detecting CD163, comprising contacting a composition suspected of containing CD163 with an antibody of the invention, under conditions effective to allow the formation of CD163/antibody complexes and detecting the complexes so formed.

Compositions comprising at least a first antibody (or binding protein) of the invention constitute a further aspect of the present invention. Formulations (compositions) comprising one or more antibodies of the invention in admixture with a suitable diluent, carrier or excipient constitute a preferred embodiment of the present invention. Such formulations may be for pharmaceutical use, e.g. veterinary use, and thus compositions of the invention are preferably pharmaceutically acceptable or acceptable for administration to non-human animals, e.g. mammals, preferably pigs. Suitable diluents, excipients and carriers are known to the skilled man.

The compositions according to the invention may be presented, for example, in a form suitable for oral, nasal, parenteral, intravenous, topical or rectal administration.

The active compounds (e.g. the antibodies of the invention) as defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions may then be filled into injection vials or ampoules.

Suitable dosage units can be determined by a person skilled in the art.

The pharmaceutical compositions may additionally comprise further active ingredients (e.g. as described elsewhere herein) in the context of co-administration regimens or combined regimens.

A further aspect of the present invention provides the anti-CD163 antibodies (or binding proteins) defined herein for use in therapy, in particular for use in the treatment or prevention of any disease or condition associated with CD163 or where CD163 has a role, for example a causative (e.g. a wholly or partially causative role) or an essential role. For example, the anti-CD163 antibodies of the invention can be used in the treatment or prevention of any infection caused by a virus or other pathogen, wherein said infection is associated with CD163, or where CD163 has a role, for example a causative (e.g. a wholly or partially causative role), or an essential role. Put another way, in accordance with the present invention the anti-CD163 antibodies (or binding proteins) may target and inhibit or reduce the function of CD163, in particular CD163 expressed on or in PAMs or other CD163 positive cells. Thus, the anti-CD163 antibodies (or binding proteins) defined herein may be used in the treatment or prevention of any disease or condition where inhibition of CD163 or blockade or reduction of CD163 function is useful.

Preferred embodiments provide the anti-CD163 antibodies (or binding proteins) of the invention for use in the treatment or prevention of infections in pigs, preferably virus infection in pigs. Particularly preferred is the treatment or prevention of PRRSV infection. In embodiments where pigs are treated, the anti-CD163 antibodies (or binding proteins) of the invention are typically anti-porcine CD163 antibodies (or binding proteins).

CD163 is believed to be the likely receptor for all PRRS viruses. However, as described elsewhere herein, there are two serotypes of PRRSV; type 1 and type 2 viruses. Although the type 1 and type 2 viruses are phenotypically similar at several levels, there are differences in the viral genotypes. The antibodies (or binding proteins) of the invention can be used to treat or prevent type 1 and/or type 2 PRRS viruses, for example type 1 and type 2 PRRS viruses. In other embodiments the present invention provides antibodies (or binding proteins) which have the ability to inhibit type 2 PRRSV infection, preferably to specifically inhibit type 2 PRRSV infection, and therefore can be used to treat or prevent type 2 PRRS viruses.

The administration of the binding proteins or antibodies in the therapeutic methods and uses of the invention is carried out in pharmaceutically, therapeutically, or physiologically effective amounts, to subjects (animals, or mammals, e.g. pigs) in need of treatment. Thus, said methods and uses may involve the additional step of identifying a subject in need of treatment.

Treatment of diseases or conditions in accordance with the present invention (for example treatment of pre-existing disease) includes cure of said disease or condition, or any reduction or alleviation of disease (e.g. reduction in disease severity) or symptoms of disease.

The therapeutic methods and uses of the prevent invention are suitable for prevention of diseases as well as active treatment of diseases (for example treatment of pre-existing disease). Thus, prophylactic and metaphylactic (treating in the face of a disease outbreak, for example treating a group of subjects after the diagnosis of infection and/or clinical disease in part of the group, with the aim of preventing the spread of infectious disease to animals in close contact and/or at significant risk) treatment is also encompassed by the invention. For this reason in the methods and uses of the present invention, treatment also includes prophylaxis, metaphylaxis or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

Clinical symptoms of for example PRRS infection include foetal reabsorption, still-births and late-term abortion in pregnant sows or gilts, and respiratory diseases and syndromes, e.g. respiratory distress, in all pigs, and especially young pigs and piglets. Other symptoms include lack of appetite (which often leads to decreased growth rates), fever, lethargy, respiratory distress, reproductive failure and diarrhoea (especially in young piglets) and Central Nervous System (CNS) signs. Subjects with PRRSV infection also have susceptibility to endemic diseases such as meningitis, Glassers disease, exudative dermatitis, sarcoptic mange and bacterial bronchopneumonia is commonly reported as increasing (Diseases of Swine, Eleventh Edition, Editor(s): Jeffrey J. Zimmerman Locke A. Karriker Alejandro Ramirez Kent J. Schwartz Gregory W. Stevenson Jianqiang Zhang, First published:29 Mar. 2019), and such diseases are generally managed by the use of antimicrobial products such as antibiotics. Consequently, the invention has a role in the reduction of antimicrobial product use on-farm.

Thus, the antibodies of the invention can be used to treat or prevent clinical disease or symptoms, e.g. clinical disease or symptoms associated with PRRSV infection or downstream endemic diseases such as those outlined above, or to reduce virus, e.g. PRRSV, circulation (e.g. numbers or titres of circulating viral particles) or to prevent infection (e.g. first infection) or new infection (e.g. second or subsequent infection), e.g. PRRSV infection (e.g. first PRRSV infection) or new PRRSV infection (e.g. second or subsequent PRRSV infection).

Preferred subjects for treatment in accordance with the present invention thus include all types of pigs (also sometimes referred to as swine), for example any pig, swine, or porcine species, including pigs of all ages and species providing they are susceptible to or are capable of being infected with pathogens as defined herein, and in particular PRRSV. Piglets, especially young piglets or live-born piglets from infected sows (up to 80% of which will die), are particularly preferred subjects, as are nursery pigs (post-weaned pigs that are for example up to 12 weeks old), and growing or fattening pigs (e.g. pigs up to the age of slaughter), in particular growing pigs. Pre-weaned piglets, e.g. piglets up to 4 weeks old (especially those of infected sows, where the infection may be transmitted via the mammary gland secretions of an infected sow) are also preferred subjects to be treated, as are sows and pregnant sows.

In some embodiments, e.g. where prevention is concerned, the subject is a subject at risk of being affected by the disease or condition in question, for example at risk of being infected with a pathogen or virus (e.g. PRRSV) as described above and developing disease. Such a subject may be a healthy subject or a subject not displaying any symptoms of disease or any other appropriate “at risk” subject. In another embodiment the subject is a subject having, or suspected of having (or developing), or potentially having (or developing) the disease or condition in question as described above.

Alternatively viewed, the present invention provides a method of treating or preventing a disease or condition associated with CD163 or where CD163 has a role, for example a causative (e.g. a wholly or partially causative role) or an essential role, which method comprises administering to a subject in need thereof a therapeutically effective amount of an anti-CD163 antibody (or binding protein) of the invention as defined herein. Appropriate diseases or conditions are described elsewhere herein.

The treatment or prevention of infections in pigs, preferably virus infection in pigs is preferred. Particularly preferred is the treatment or prevention of PRRSV infection, e.g. to treat or prevent type 1 and/or type 2 PRRS virus infection, for example type 1 and type 2 PRRS virus infection, or to treat or prevent (e.g. specifically treat or prevent) type 2 PRRS virus infection.

Thus, a yet further aspect provides a method of treatment or prevention of PRRSV infection in a pig, e.g. treatment or prevention of type 1 and/or type 2 PRRS virus infection in a pig, which method comprises administering to a subject in need thereof a therapeutically effective amount of a monoclonal antibody which binds to porcine CD163. Appropriate CD163 antibodies (or binding proteins) for use in such methods are described herein.

Embodiments of the therapeutic uses of the invention described herein apply, mutatis mutandis, to this aspect of the invention.

A therapeutically effective amount will be determined based on the clinical assessment and can be readily monitored.

Further alternatively viewed, the present invention provides the use of an anti-CD163 antibody (or binding protein) of the invention, e.g. a monoclonal antibody of the invention, as defined herein in the manufacture of a medicament for use in therapy. Preferred therapeutic uses are described elsewhere herein, in particular for use in the treatment or prevention of any disease or condition associated with CD163 or where CD163 has a role, for example a causative (e.g. a wholly or partially causative role) or an essential role. For example, the anti-CD163 antibodies (or binding proteins) of the invention can be used in the treatment or prevention of any infection caused by a virus or other pathogen, wherein said infection is associated with CD163, or where CD163 has a role, for example a causative (e.g. a wholly or partially causative role), or an essential role. Put another way, in accordance with the present invention the anti-CD163 antibodies (or binding proteins) may target and inhibit or reduce the function of CD163, in particular CD163 expressed on or in PAMs or other CD163 positive cells. Thus, the anti-CD163 antibodies (or binding proteins) defined herein may be used in the treatment or prevention of any disease or condition where inhibition of CD163 or blockade or reduction of CD163 function is useful.

Preferred embodiments provide the use of anti-CD163 antibodies (or binding proteins) of the invention in the manufacture of a medicament for use in the treatment or prevention of infections in pigs, preferably virus infection in pigs. Particularly preferred is the treatment or prevention of PRRSV infection, e.g. to treat or prevent type 1 and/or type 2 PRRS virus infection, for example type 1 and type 2 PRRS virus infection, or to treat or prevent (e.g. specifically treat or prevent) type 2 PRRS virus infection.

Thus, a yet further aspect provides the use of a monoclonal antibody which binds to porcine CD163, in the manufacture of a medicament for use in the treatment or prevention of PRRS virus infection, preferably type 1 and/or type 2 PRRS virus infection in a pig. Appropriate CD163 antibodies (or binding proteins) for such uses are described herein.

Embodiments of the therapeutic uses of the invention described herein apply, mutatis mutandis, to this aspect of the invention.

In some embodiments, the antibodies of the invention can be used in combination.

Any combination of the VHH antibodies 49(#18), 47(#19), 48(#20), 76(#2), 77(#16), 78(#8), 150(#15), 70(#23), and 144(#1), as shown in Tables A, B, C, D, E, F, G, H, and I, respectively, can be used. Thus 2, 3, 4, 5, 6, 7, 8 or all 9 of these can be used in combination, preferably 2 or 3, more preferably 2.

Preferred combinations comprise:
70(#23) and one or more, preferably one of 76(#2), 78(#8), 77(#16), 49(#18), 47(#19), or 48(#20);
144(#1) and one or more, preferably one of 76(#2), 78(#8), 77(#16), 49(#18), 47(#19), or 48(#20);
150(#15) and one or more, preferably one of 76(#2), 78(#8), 77(#16), 49(#18), 47(#19), or 48(#20), e.g. 150(#15) and 47(#19);
76(#2) and one or more, preferably one of 70(#23) or 144(#1) or 150(#15);
77(#16) and one or more, preferably one of 70(#23) or 144(#1) or 150(#15);
49(#18) and one or more, preferably one of 70(#23) or 144(#1) or 150(#15);
47(#19) and one or more, preferably one of 70(#23) or 144(#1) or 150(#15);
48(#20) and one or more, preferably one of 70(#23) or 144(#1) or 150(#15); or
78(#8) and one or more, preferably one of 70(#23) or 144(#1) or 150(#15).

Preferred combinations comprise:
76(#2) and 150(#15);
76(#2) and 77(#16);
76(#2) and 48(#20);
150(#15) and 77(#16);
150(#15) and 48(#20);
77(#16) and 48(#20); or
150(#15) and 77(#16) and 48(#20).

Any combination of the VHH antibodies 57(#11), 41(#12), 171(#14), and 29(#17), as shown in Tables 1, 2, 3 and 4, respectively, can be used in the invention. Thus 2, 3, or all 4 of these can be used in combination, preferably 2 or 3, more preferably 2.

Preferred combinations comprise:
57(#11) and one or more, preferably one of 41(#12) or 29(#17); e.g. 57(#11) and 29(#17);
171(#14) and one or more, preferably one of 41(#12) or 29(#17);
41 (#12) and one or more, preferably one of 57(#11) or 171 (#14); or
29(#17) and one or more, preferably one of 57(#11) or 171(#14).

Other combinations include:
Any combination of one or more, preferably one, of the VHH antibodies 49(#18), 47(#19), 48(#20), 76(#2), 77(#16), 78(#8), 150(#15), 70(#23), or 144(#1), as shown in Tables A, B, C, D, E, F, G, H, and I, respectively, with one or more, preferably one, of the VHH antibodies 57(#11), 41(#12), 171(#14), or 29(#17), as shown in Tables 1, 2, 3 and 4, respectively.

Preferred combinations comprise:
150(#15) and 29(#17);
47(#19) and 29(#17); or
144(#1) and 29(#17).

Other preferred combinations comprise:
57(#11) and one or more, preferably one of 41 (#12) or 29(#17);
57(#11) and one or more, preferably one of 76(#2), 78(#8), 77(#16), 49(#18), 47(#19), or 48(#20);
171(#14) and one or more, preferably one of 41(#12) or 29(#17);
171(#14) and one or more, preferably one of 76(#2), 78(#8), 77(#16), 49(#18), 47(#19), or 48(#20);
70(#23) and one or more, preferably one of 41 (#12) or 29(#17);
70(#23) and one or more, preferably one of 76(#2), 78(#8), 77(#16), 49(#18), 47(#19), or 48(#20);
144(#1) and one or more, preferably one of 41(#12) or 29(#17);
144(#1) and one or more, preferably one of 76(#2), 78(#8), 77(#16), 49(#18), 47(#19), or 48(#20);
150(#15) and one or more, preferably one of 41(#12) or 29(#17); or
150(#15) and one or more, preferably one of 76(#2), 78(#8), 77(#16), 49(#18), 47(#19), or 48(#20).

Alternative preferred combinations comprise:
41(#12) and one or more, preferably one of 57(#11), 171 (#14), 70(#23), 144(#1), or 150(#15);
41 (#12) and one or more, preferably one of 76(#2), 78(#8), 77(#16), 49(#18), 47(#19), or 48(#20);
29(#17) and one or more, preferably one of 57(#11), 171(#14), 70(#23), 144(#1) or 150(#15); or
29(#17) and one or more, preferably one of 76(#2), 78(#8), 77(#16), 49(#18), 47(#19), or 48(#20).

Alternative preferred combinations comprise:
76(#2) and one or more, preferably one of 57(#11), 171(#14), 70(#23), 144(#1) or 150(#15);
76(#2) and one or more, preferably one of 41(#12) or 29(#17);
77(#16) and one or more, preferably one of 57(#11), 171(#14), 70(#23), 144(#1) or 150(#15);
77(#16) and one or more, preferably one of 41(#12) or 29(#17);
49(#18) and one or more, preferably one of 57(#11), 171(#14), 70(#23), 144(#1) or 150(#15);
49(#18) and one or more, preferably one of 41(#12) or 29(#17);
47(#19) and one or more, preferably one of 57(#11), 171(#14), 70(#23), 144(#1) or 150(#15);
47(#19) and one or more, preferably one of 41(#12) or 29(#17);
48(#20) and one or more, preferably one of 57(#11), 171(#14), 70(#23), 144(#1) or 150(#15);
48(#20) and one or more, preferably one of 41(#12) or 29(#17);
78(#8) and one or more, preferably one of 57(#11), 171(#14), 70(#23), 144(#1) or 150(#15); or
78(#8) and one or more, preferably one of 41(#12) or 29(#17).

Preferred combinations comprise:
57(#11) and one or more, preferably one of 76(#2), 78(#8), 77(#16), 49(#18), 47(#19), or 48(#20);
171(#14) and one or more, preferably one of 76(#2), 78(#8), 77(#16), 49(#18), 47(#19) or 48(#20);
70(#23) and one or more, preferably one of 41(#12) or 29(#17);
144(#1) and one or more, preferably one of 41(#12) or 29(#17);
150(#15) and one or more, preferably one of 41(#12) or 29(#17);
41(#12) and one or more, preferably one of 70(#23), 144(#1) or 150(#15);
41(#12) and one or more, preferably one of 76(#2), 78(#8), 77(#16), 49(#18), 47(#19) or 48(#20);
29(#17) and one or more, preferably one of 70(#23),144(#1) or 150(#15);
29(#17) and one or more, preferably one of 76(#2), 78(#8), 77(#16), 49(#18), 47(#19) or 48(#20);
76(#2) and one or more, preferably one of 57(#11) or 171(#14);
76(#2) and one or more, preferably one of 41(#12) or 29(#17);
77(#16) and one or more, preferably one of 57(#11) or 171(#14);
77(#16) and one or more, preferably one of 41(#12) or 29(#17);
49(#18) and one or more, preferably one of 57(#11) or 171(#14);
49(#18) and one or more, preferably one of 41(#12) or 29(#17);
47(#19) and one or more, preferably one of 57(#11) or 171(#14);
47(#19) and one or more, preferably one of 41(#12) or 29(#17);
48(#20) and one or more, preferably one of 57(#11) or 171(#14);
48(#20) and one or more, preferably one of 41(#12) or 29(#17);
78(#8), and one or more, preferably one of 57(#11) or 171(#14); or
78(#8), and one or more, preferably one of 41 (#12) or 29(#17).
In all the above combinations, antibodies with the 3 CDRs as shown in Tables A to I and 1 to 4, as appropriate, can also be used.

Preferred combinations are those that result in improved or increased, preferably significantly improved or increased, therapeutic efficacy as compared to any of the antibodies of the invention (e.g. VHHs) administered as a sole active agent (monotherapy), or sole antibody, or sole anti-CD163 agent. Other preferred combinations are those where the individual anti-CD163 antibodies of the combination bind to different epitopes on the CD163 molecule.

For such combination treatments using two or more antibodies (or binding proteins) of the invention, the second (or subsequent) anti-CD163 antibody may be administered to a subject substantially simultaneously with the first anti-CD163 antibody of the invention, such as from a single pharmaceutical composition or from two pharmaceutical compositions administered closely together (at the same or a similar time). Alternatively, the second (or subsequent) anti-CD163 antibody of the invention may be administered to a subject at a time prior to or sequential to the administration of the first anti-CD163 antibody of the invention. “At a time prior to or sequential to”, as used herein, means “staggered”, such that the second antibody is administered to a subject at a time distinct to the administration of the first anti-CD163 antibody component. Generally, the two (or more) components may be administered at times effectively spaced apart or together to allow the individual components to exert their respective therapeutic effects, i.e., they are administered in “biologically effective amounts” at “biologically effective time intervals” and are administered as part of the same therapeutic regimen.

Combinations of anti-CD163 antibodies (or binding proteins) of the invention, can, if appropriate, conveniently be administered as part of the same molecule or construct, e.g. can be conjugated or linked together, e.g. with an artificial linker. This mode of administration can be particularly appropriate for VHH antibodies (or other types of antibody molecule which are composed of a single polypeptide chain), individual antibodies of which can conveniently be connected by appropriate peptide (or other) linkers, e.g. non-native peptide or artificial linkers, in a single polypeptide chain containing multiple VHH (or other) antibodies, either of the invention or in combination with other VHHs or other antibodies. In such embodiments, agents are generally linked together using appropriate techniques, e.g. spacing, such that each component can exert their respective effects, for example binding to CD163. For example, in embodiments where anti-CD163 antibodies of the invention bind to different epitopes on CD163, then combinations of such antibodies are preferred and the constructs are designed appropriately so that each individual antibody can bind to CD163, e.g. to its CD163 epitope.

Thus, in some embodiments the anti-CD163 antibodies (or binding proteins) of the invention may be used as the sole active agent in a treatment regimen (monotherapy), or more than one of anti-CD163 antibodies of the invention can be used in combination, for example as described above. In some embodiments the anti-CD163 antibodies (or binding proteins) of the invention (or combinations as appropriate) may be used as the sole active anti-CD163 agent(s) or the sole active anti-CD163 antibodies in a treatment regimen, or they may be the sole active anti-PRRSV agent(s) in a treatment regimen. However, in some embodiments, additional anti-CD163 agents or anti-PRRSV agents can be used.

Thus, the anti-CD163 binding proteins or antibodies of the invention (or combination as appropriate) can be combined with one or more further (additional CD163 targeting or non-CD163 targeting) active agents, e.g. with at least a second therapeutic or biological agent, where the anti-CD163 binding protein or antibody of the invention (or combination of such binding proteins or antibodies), is the first.

The anti-CD163 antibodies (or binding proteins) of the invention (or combination as appropriate) can for example be combined with any other therapeutic agent which is useful to treat the disease in question as described elsewhere herein, for example PRRSV.

For such combination treatments, the second (non-anti-CD163 antibody of the invention) agent may be, speaking generally, administered to a subject substantially simultaneously with the anti-CD163 antibody of the invention (or combination of such antibodies), such as from a single pharmaceutical composition or from two pharmaceutical compositions administered closely together (at the same or a similar time). Alternatively, the second (non-anti-CD163 antibody of the invention) agent may be administered to a subject at a time prior to or sequential to the administration of the anti-CD163 antibody of the invention component. “At a time prior to or sequential to”, as used herein, means “staggered”, such that the second (non-anti-CD163 antibody of the invention) agent is administered to a subject at a time distinct to the administration of the anti-CD163 antibody component. Generally, the two (or more) components may be administered at times effectively spaced apart or together to allow the two components to exert their respective therapeutic effects, i.e., they are administered in “biologically effective amounts” at “biologically effective time intervals” and are administered as part of the same therapeutic regimen.

The invention further includes kits comprising one or more of the antibodies, or compositions of the invention, or one or more of the nucleic acid molecules encoding the antibodies of the invention, or one or more recombinant expression vectors comprising the nucleic acid sequences of the invention, or one or more host cells or viruses comprising the recombinant expression vectors or nucleic acid sequences of the invention. Preferably said kits are for use in the methods and uses as described herein, e.g. the therapeutic methods as described herein. Preferably said kits comprise instructions for use of the kit components. Preferably said kits are for treating diseases or conditions as described elsewhere herein, and optionally comprise instructions for use of the kit components to treat such diseases or conditions. Equivalent embodiments with binding proteins of the invention are also provided.

The antibodies (or binding proteins) of the invention as defined herein may also be used as molecular tools for in vitro or in vivo applications and assays. As the antibodies (and some binding proteins) have an antigen binding site, these can function as members of specific binding pairs and these molecules can be used in any assay where the particular binding pair member is required.

Thus, yet further aspects of the invention provide a reagent that comprises an antibody (or binding proteins) of the invention as defined herein and the use of such antibodies (or binding proteins) as molecular tools, for example in in vitro or in vivo assays.

### TABLES OF AMINO ACID SEQUENCES DISCLOSED HEREIN AND THEIR SEQUENCE IDENTIFIERS (SEQ ID NOs)

All amino acid sequences are recited herein from the N-terminus to the C-terminus in line with convention in this technical field.

TABLE A

| SEQ ID NO: | Description | Sequence |
|---|---|---|
| VHCDR3 49(#18); VHH 018 (3E01) | | |
| 1 | VHH (aa) | QVQLQESGGGLVQVGGSLRLSCVASGRAPSRYVMGWFRQAPGQEREFVAGIAWSGRAPYADSVKGRSTISRDNAKNTVYLQMNSLKPEDTGVYYCAGGEGAIRWTTLDAYDYWGQGTQVTVSS |
| 2 | Heavy CDR1 | RYVMG |
| 3 | Heavy CDR2 | GIAWSGRAPYADSVKG |
| 4 | Heavy CDR3 | GEGAIRWTTLDAYDY |
| 5 | Heavy FR1 | QVQLQESGGGLVQVGGSLRLSCVASGRAPS |
| 6 | Heavy FR2 | WFRQAPGQEREFVA |
| 7 | Heavy FR3 | RSTISRDNAKNTVYLQMNSLKPEDTGVYYCAG |
| 8 | Heavy FR4 | WGQGTQVTVSS |

TABLE B

| SEQ ID NO: | Description | Sequence |
|---|---|---|
| VHCDR3 47(#19); VHH 019 (3E11) | | |
| 9 | VHH (aa) | QVQLQESGGGLVQVGSSLRLSCVTSGRTPSRYVMGWFRQAPGQEREFVAAISWSGRAPYADSVKGRFTISRDNAKNTVYLQMNSLKPEDTGVYYCAGGEGAIKWTTLDAYDYWGQGTQVTVSS |
| 10 | Heavy CDR1 | RYVMG |
| 11 | Heavy CDR2 | AISWSGRAPYADSVKG |
| 12 | Heavy CDR3 | GEGAIKWTTLDAYDY |
| 13 | Heavy FR1 | QVQLQESGGGLVQVGSSLRLSCVTSGRTPS |
| 14 | Heavy FR2 | WFRQAPGQEREFVA |
| 15 | Heavy FR3 | RFTISRDNAKNTVYLQMNSLKPEDTGVYYCAG |
| 16 | Heavy FR4 | WGQGTQVTVSS |

TABLE C

| SEQ ID NO: | Description | Sequence |
|---|---|---|
| VHCDR3 48(#20); VHH 020 (3H11) | | |
| 17 | VHH (aa) | QVQLQESGGGLVQVGGSLRLSCVASGRTPSRYVMGWFRQAPGQEREFVAGIAWSGRAPYADSVKGRFVISRDSAKNTVYLQMNSLKSEDTGVYYCAGGEGAILWTTPGAYNYWGQGTQVTVSS |
| 18 | Heavy CDR1 | RYVMG |
| 19 | Heavy CDR2 | GIAWSGRAPYADSVKG |
| 20 | Heavy CDR3 | GEGAILWTTPGAYNY |
| 21 | Heavy FR1 | QVQLQESGGGLVQVGGSLRLSCVASGRTPS |
| 22 | Heavy FR2 | WFRQAPGQEREFVA |
| 23 | Heavy FR3 | RFVISRDSAKNTVYLQMNSLKSEDTGVYYCAG |
| 24 | Heavy FR4 | WGQGTQVTVSS |

TABLE D

| SEQ ID NO: | Description | Sequence |
|---|---|---|
| VHCDR3 76(#2); VHH 002 (1B04) | | |
| 25 | VHH (aa) | QVQLVESGGGLVQAGGSLRLSCAASGLTFVTYSMGWFRQAPGKEREFVAAHRWSGSAYYAEHADSVEGRFTISRDYAKNMLYLQMNSLKHEDTAVYYCAAGVGSAAQYRYWGRGTQVTVSS |
| 26 | Heavy CDR1 | TYSMG |
| 27 | Heavy CDR2 | AHRWSGSAYYAEHADSVEG |
| 28 | Heavy CDR3 | GVGSAAQYRY |
| 29 | Heavy FR1 | QVQLVESGGGLVQAGGSLRLSCAASGLTFV |
| 30 | Heavy FR2 | WFRQAPGKEREFVA |
| 31 | Heavy FR3 | RFTISRDYAKNMLYLQMNSLKHEDTAVYYCAA |
| 32 | Heavy FR4 | WGRGTQVTVSS |

TABLE E

| SEQ ID NO: | Description | Sequence |
|---|---|---|
| VHCDR3 77(#16); VHH 016 (2H11) | | |
| 33 | VHH (aa) | EVQLVESGGGLVQAGGSLRLSCAASGRTFAPGSMGWFRQAPGKEREFVAAHRWSGSAYYADYADSVEGRFTISRDYAKNMVYLQMNSLKPGDTAVYYCAAGVGSAAQYTYWGRGTQVTVSS |
| 34 | Heavy CDR1 | PGSMG |
| 35 | Heavy CDR2 | AHRWSGSAYYADYADSVEG |
| 36 | Heavy CDR3 | GVGSAAQYTY |
| 37 | Heavy FR1 | EVQLVESGGGLVQAGGSLRLSCAASGRTFA |
| 38 | Heavy FR2 | WFRQAPGKEREFVA |
| 39 | Heavy FR3 | RFTISRDYAKNMVYLQMNSLKPGDTAVYYCAA |
| 40 | Heavy FR4 | WGRGTQVTVSS |

TABLE F

| SEQ ID NO: | Description | Sequence |
|---|---|---|
| VHCDR3 78(#8); VHH 008 (1H01) | | |
| 41 | VHH (aa) | QVQLVESGGGLVQAGGSLRLSCAASGRTFGTYSMGWFRQAPGKEREFVAAHRWSGSAYYAEHADSVEGRFTISRDYAKNMLYLQMNSLKHEDTAVYYCAAGVGSEAQYRYWGRGTQVTVSS |
| 42 | Heavy CDR1 | TYSMG |
| 43 | Heavy CDR2 | AHRWSGSAYYAEHADSVEG |
| 44 | Heavy CDR3 | GVGSEAQYRY |
| 45 | Heavy FR1 | QVQLVESGGGLVQAGGSLRLSCAASGRTFG |
| 46 | Heavy FR2 | WFRQAPGKEREFVA |
| 47 | Heavy FR3 | RFTISRDYAKNMLYLQMNSLKHEDTAVYYCAA |
| 48 | Heavy FR4 | WGRGTQVTVSS |

TABLE G

| SEQ ID NO: | Description | Sequence |
|---|---|---|
| VHCDR3 150(#15); VHH 015 (2G01) | | |
| 49 | VHH (aa) | QVQLVESGGGLVQAGDTLRLSCTASGRTFSSYSMGWFRQAPGKEREFVAAITWNGYITNYADSVKGRFTISRDNTKNTVFLQMNSLKPEETAVYYCAATTFSTTSPISRTYNYWGPGTQVTVSS |
| 50 | Heavy CDR1 | SYSMG |
| 51 | Heavy CDR2 | AITWNGYITNYADSVKG |
| 52 | Heavy CDR3 | TTFSTTSPISRTYNY |
| 53 | Heavy FR1 | QVQLVESGGGLVQAGDTLRLSCTASGRTFS |
| 54 | Heavy FR2 | WFRQAPGKEREFVA |
| 55 | Heavy FR3 | RFTISRDNTKNTVFLQMNSLKPEETAVYYCAA |
| 56 | Heavy FR4 | WGPGTQVTVSS |

TABLE H

| SEQ ID NO: | Description | Sequence |
|---|---|---|
| VHCDR3 70(#23); VHH 023 (4E10) | | |
| 57 | VHH (aa) | QVQLVESGGGLVQAGGSLRLSCVASSRTSSTYAMGWFRQGPGKERDFVAIISFGGTFYADSVKGRFTISRDNAKNTVYLQMNSLKPEDTAVYYCAAGRTLSKRADSYASWGQGTQVTVSS |
| 58 | Heavy CDR1 | TYAMG |
| 59 | Heavy CDR2 | IISFGGTFYADSVKG |
| 60 | Heavy CDR3 | GRTLSKRADSYAS |
| 61 | Heavy FR1 | QVQLVESGGGLVQAGGSLRLSCVASSRTSS |
| 62 | Heavy FR2 | WFRQGPGKERDFVA |
| 63 | Heavy FR3 | RFTISRDNAKNTVYLQMNSLKPEDTAVYYCAA |
| 64 | Heavy FR4 | WGQGTQVTVSS |

TABLE I

| SEQ ID NO: | Description | Sequence |
|---|---|---|
| VHCDR3 144(#1); VHH 001 (1B02) | | |
| 65 | VHH (aa) | EVQLVESGGGLVQAGGSLSLSCAASGGTLAMYAMSWFRQAPGKDRKFVAAINTSGRYSRYADSVKGRFTISRDNAKNTATLQMNSLEPEDTAVYYCAATDKGNWALAMSYDYWGQGTQVTVSS |
| 66 | Heavy CDR1 | MYAMS |
| 67 | Heavy CDR2 | AINTSGRYSRYADSVKG |
| 68 | Heavy CDR3 | TDKGNWALAMSYDY |
| 69 | Heavy FR1 | EVQLVESGGGLVQAGGSLSLSCAASGGTLA |
| 70 | Heavy FR2 | WFRQAPGKDRKFVA |
| 71 | Heavy FR3 | RFTISRDNAKNTATLQMNSLEPEDTAVYYCAA |
| 72 | Heavy FR4 | WGQGTQVTVSS |

TABLE J

Consensus sequences

| SEQ ID NO: | Description | Sequence |
|---|---|---|
| 73 | Heavy CDR2 | X₁ I X₃ W S G R A P Y A D S V K G |
| 74 | Heavy CDR2 | G/A I A/S W S G R A P Y A D S V K G |
| 75 | Heavy CDR3 | G E G A I X₆ WT T X₁₀ X₁₁ A Y X₁₄ Y |
| 76 | Heavy CDR3 | G E G A I R/K/L W T T L/P D/G A Y D/N Y |
| 77 | Heavy CDR1 | X₁ X₂ S M G |
| 78 | Heavy CDR1 | T/PY/G S M G |
| 79 | Heavy CDR2 | A H R W S G S A Y Y A X₁₂ X₁₃ A D S V E G |
| 80 | Heavy CDR2 | A H R W S G S A Y Y A E/D H/Y A D S V E G |
| 81 | Heavy CDR3 | G V G S X₅ A Q Y X₉ Y |
| 82 | Heavy CDR3 | G V G S A/E A Q Y R/T Y |

TABLE 1

| | | |
|---|---|---|
| SEQ ID NO: | Description | Sequence |
| VHCDR3 57(#11); VHH 011 (2B06) | | |
| 83 or 1′ | VHH (aa) | EVQLVESGGGLVQAGGSLRLSCAASGRTLSVYGTGWFRQAPGKEREFVAGISGTTGSTLYADSVKGRFTISRDNAKNTVYLQMNSLKSEDTALYYCAAGGRVYITTSSWAYWGQGTQVTVSS |
| 84 or 2′ | Heavy CDR1 | VYGTG |
| 85 or 3′ | Heavy CDR2 | GISGTTGSTLYADSVKG |
| 86 or 4′ | Heavy CDR3 | GGRVYITTSSWAY |
| 87 or 5′ | Heavy FR1 | EVQLVESGGGLVQAGGSLRCAASGRTLS |
| 88 or 6′ | Heavy FR2 | WFRQAPGKEREFVA |
| 89 or 7′ | Heavy FR3 | RFTISRDNAKNTVYLQMNSKLSEDTALYYCAA |
| 90 or 8′ | Heavy FR4 | WGQGTQVTVSS |

TABLE 2

| | | |
|---|---|---|
| SEQ ID NO: | Description | Sequence |
| VHCDR3 41(#12); VHH 012 (2C07) | | |
| 91 or 9′ | VHH (aa) | QLQLVESGGGLVQAGGSLRLSCVASGRTFSRYAMGWFRQAPGKEREFVAAIAWSTGSTYYANSVKGRFAISGDNAKNTVYLQMNSLKPEDTAVYYCAAETRYCSGFGCLDPRTYGSWGQGTQVTVSS |
| 92 or 10′ | Heavy CDR1 | RYAMG |
| 93 or 11′ | Heavy CDR2 | AIAWSTGSTYYANSVKG |
| 94 or 12′ | Heavy CDR3 | ETRYCSGFGCLDPRTYGS |
| 95 or 13′ | Heavy FR1 | QLQLVESGGGLVQAGGSLRLSCVASGRTFS |
| 96 or 14′ | Heavy FR2 | WFRQAPGKEREFVA |
| 97 or 15′ | Heavy FR3 | RFAISGDNAKNTVYLQMNSLKPEDTAVYYCAA |
| 98 or 16′ | Heavy FR4 | WGQGTQVTVSS |

TABLE 3

| | | |
|---|---|---|
| SEQ ID NO: | Description | Sequence |
| VHCDR3 171(#14); VHH 014 (2D01) | | |
| 99 or 17′ | VHH (aa) | QLQLVESGGGLVQPGGSLRLSCAASGRTFSTDTMAWFRQAPGKEREFIAGIGRSGGSIYYAKAVKGRFTVSRDNAKNTVYLQMNSLKAEDTAVYYCAARQRIGLVVGALGYDYWGQGTQVTVSS |
| 100 or 18′ | Heavy CDR1 | TDTMA |
| 101 or 19′ | Heavy CDR2 | GIGRSGGSIYYADAVKG |
| 102 or 20′ | Heavy CDR3 | RQRIGLVVGALGYDY |
| 103 or 21′ | Heavy FR1 | QLQLVESGGGLVQPGGSLRLSCAASGRTFS |
| 104 or 22’ | Heavy FR2 | WFRQAPGKEREFIA |
| 105 or 23’ | Heavy FR3 | RFTVSRDNAKNTVYLQMNSLKAEDTAVYYCAA |
| 106 or 24′ | Heavy FR4 | WGQGTQVTVSS |

TABLE 4

| | | |
|---|---|---|
| SEQ ID NO: | Description | Sequence |
| VHCDR3 29(#17); VHH 017 (3D03) | | |
| 107 or 25′ | VHH (aa) | QLQLVESGGGLVQPGGSLRLSCAASGFTLDDYTIGWFRQAPGKEREGVSCINSITSNTYYADSVKGRFTISRDNAKNTVYLQMNSLTAEDTAIYYCAADSGLFSGSSCLKYRAMRFGSWGQGTQVTVSS |
| 108 or 26′ | Heavy CDR1 | DYTIG |
| 109 or 27′ | Heavy CDR2 | CINSITSNTYYADSVKG |
| 110 or 28′ | Heavy CDR3 | DSGLFSGSSCLKYRAMRFGS |
| 111 or 29′ | Heavy FR1 | QLQLVESGGGLVQPGGSLRLSCAASGFTLD |
| 112 or 30′ | Heavy FR2 | WFRQAPGKEREGVS |
| 113 or 31′ | Heavy FR3 | RFTISRDNAKNTVYLQMNSLTAEDTAIYYCAA |
| 114 or 32′ | Heavy FR4 | WGQGTQVTVSS |

The invention will now be further described in the following non-limiting Examples with reference to the following drawings:

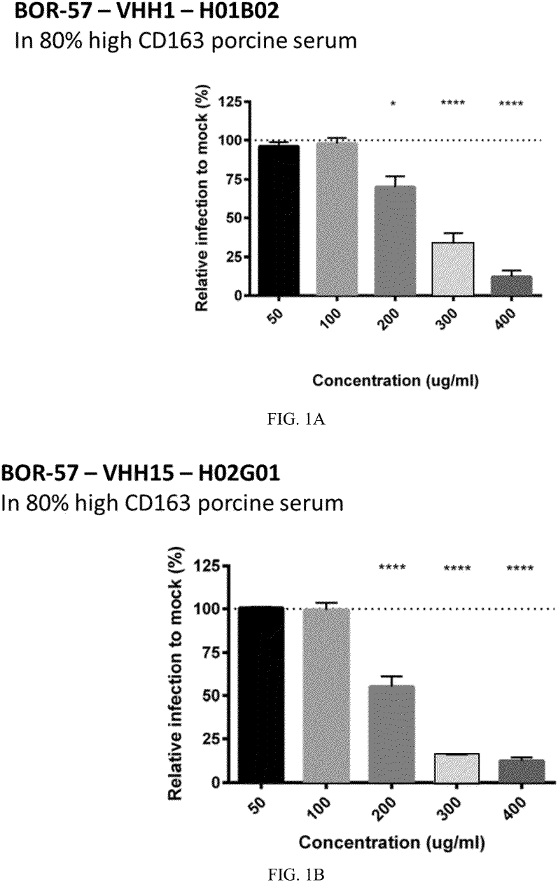

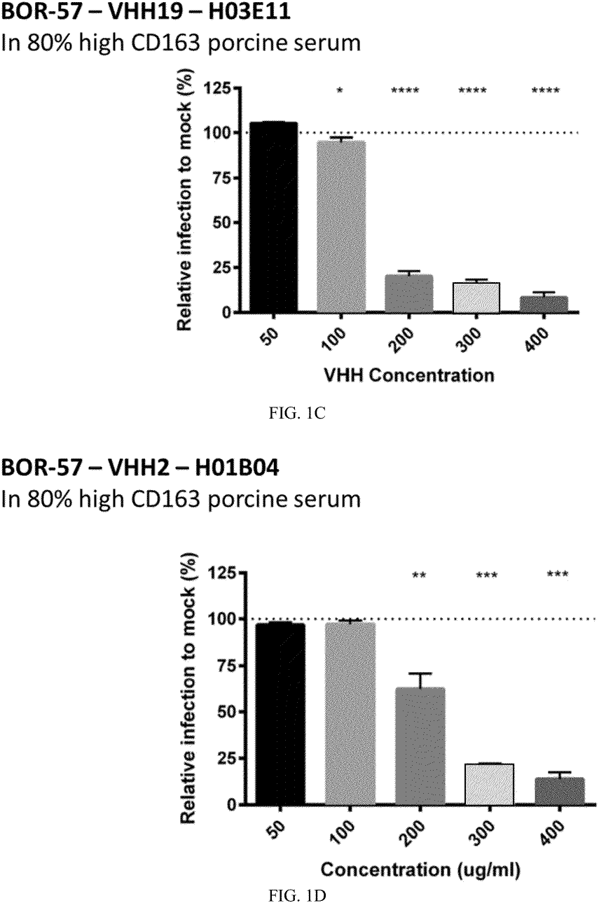

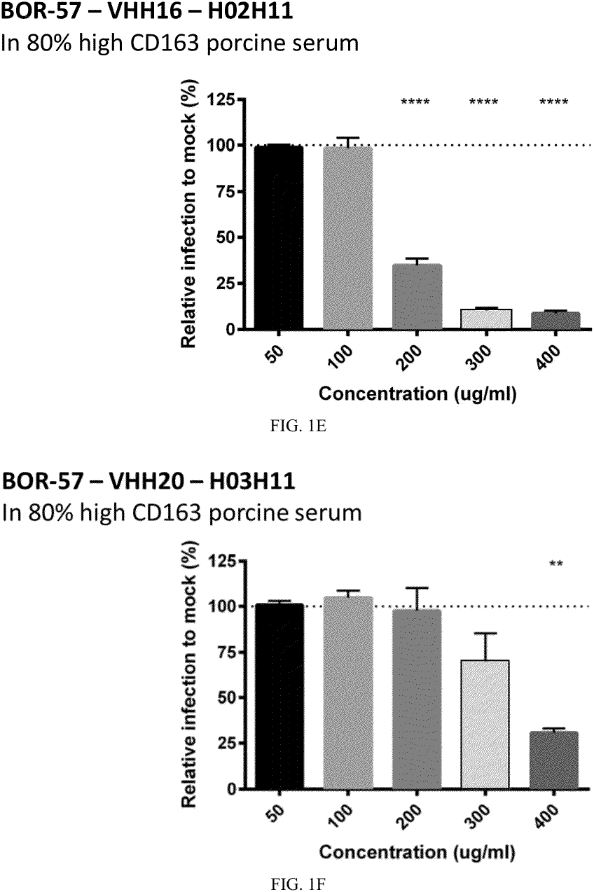

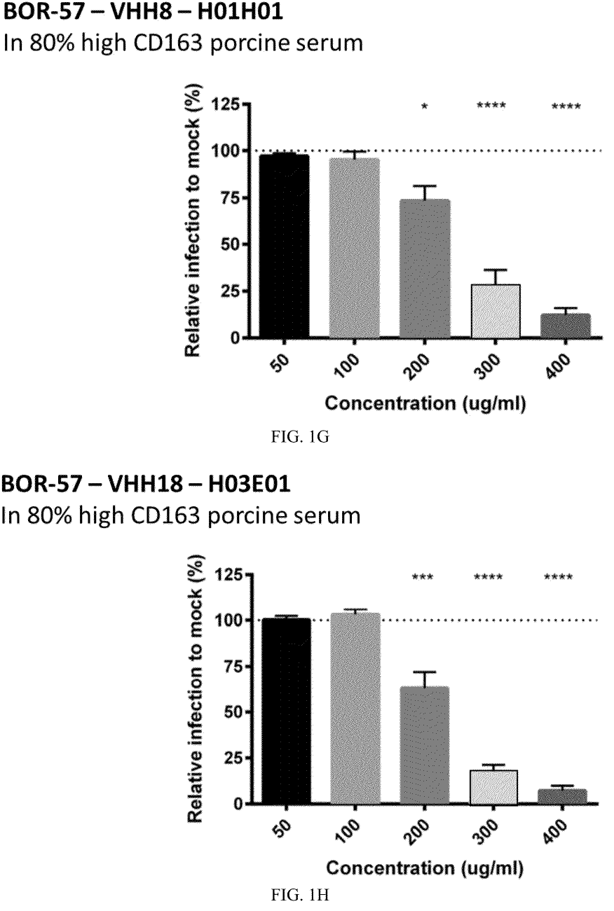

FIG. 1: Blockade of PRRS Type-1 virus BOR57 in presence of high porcine serum The data in panels A to I demonstrates the ability of candidate VHH antibodies to inhibit productive viral infection by Type 1 BOR57 PRRS virus when incubated with porcine alveolar macrophage cells for 17 hours in the presence of 80% porcine serum. Candidate VHH antibodies were assessed over a dose response range between 50 to 400 µg/mL. Data are presented relative to an infection in the absence of the test article compared to a mock control as presented as mean +/- SEM (n>3).

FIG. 2: Blockade of PRRS Type-2 virus MN184 in 10% FBS containing medium Data demonstrates the ability of candidate VHH antibodies to inhibit productive viral infection by Type 2 MN184 PRRS virus when incubated with porcine alveolar macrophage cells for 17 hours in the presence of 10% FBS. Candidate antibodies were assessed over a dose response range between 50 to 300 µg/mL. Data are presented relative to an infection in the absence of the test article compared to a mock control as presented as mean +/- SEM (n>3).

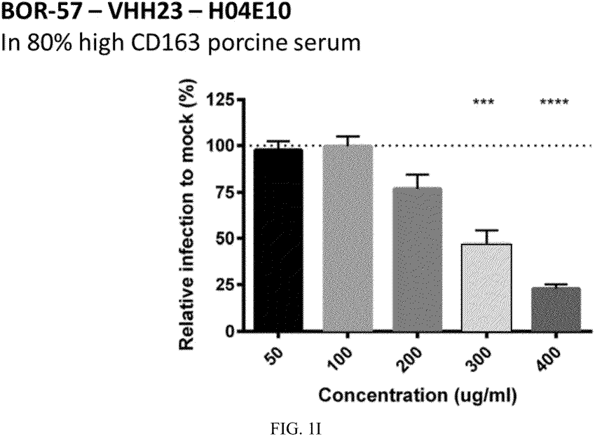

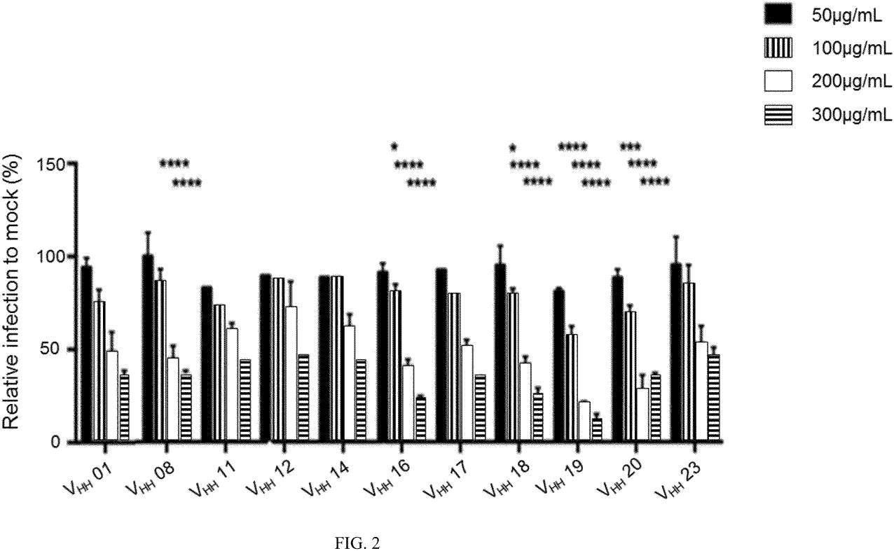

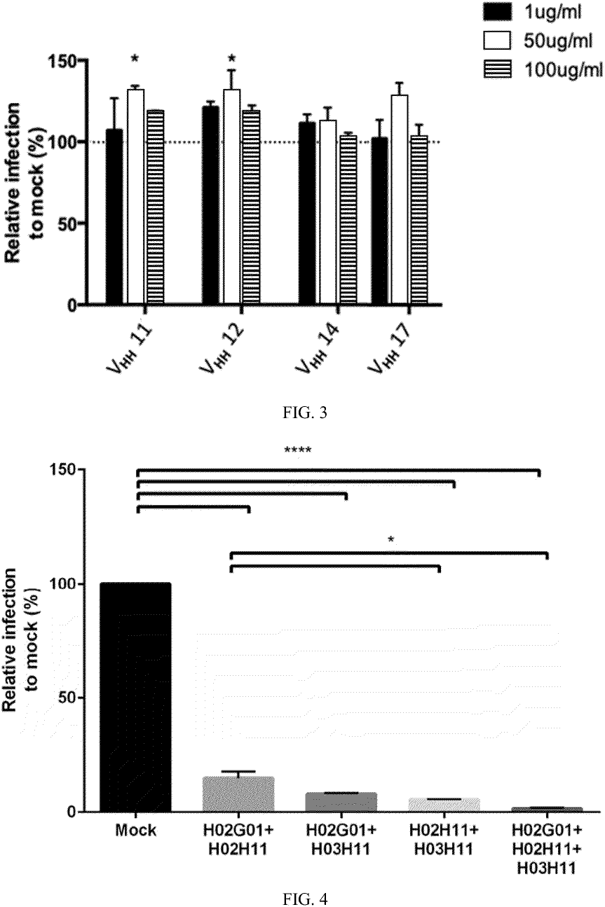

FIG. 3: Blockade of Type 1 Virus Infection by PRRS Virus Type-2 specific VHH Inhibitory Antibodies Data demonstrates the ability of candidate VHH antibodies to inhibit productive viral infection by Type 1 BOR57 PRRS virus when incubated with porcine alveolar macrophage cells for 17 hours in the presence of 80% porcine serum. Candidate antibodies were assessed over a dose response range between 1 to 100 µg/mL. Data are presented relative to an infection in the absence of the test article compared to a mock control as presented as mean +/- SEM (n>3).

FIG. 4: Blockade of PRRS Type-1 virus BOR57 using VHH combinations comprising VHH15, VHH16 and VHH20 The data show that individual VHH may be used in combination to block infection by PRRS Type-1 virus. A combination of VHH15 and VHH16, VHH15 and VHH20, as well as VHH16 and VHH20 showed effective blockade of productive virus infection. A single concentration of 100 µg/mL of each VHH of the VHH pair was tested. Additionally, a triple combination of VHH15+VHH16+VHH20 was assessed, with each VHH present at a concentration of 50 µg/mL. The data demonstrate the potential for combinations of VHH to be used to block infection by PRRS virus. Data are shown relative to an infection in the absence of the test article compared to a mock control.

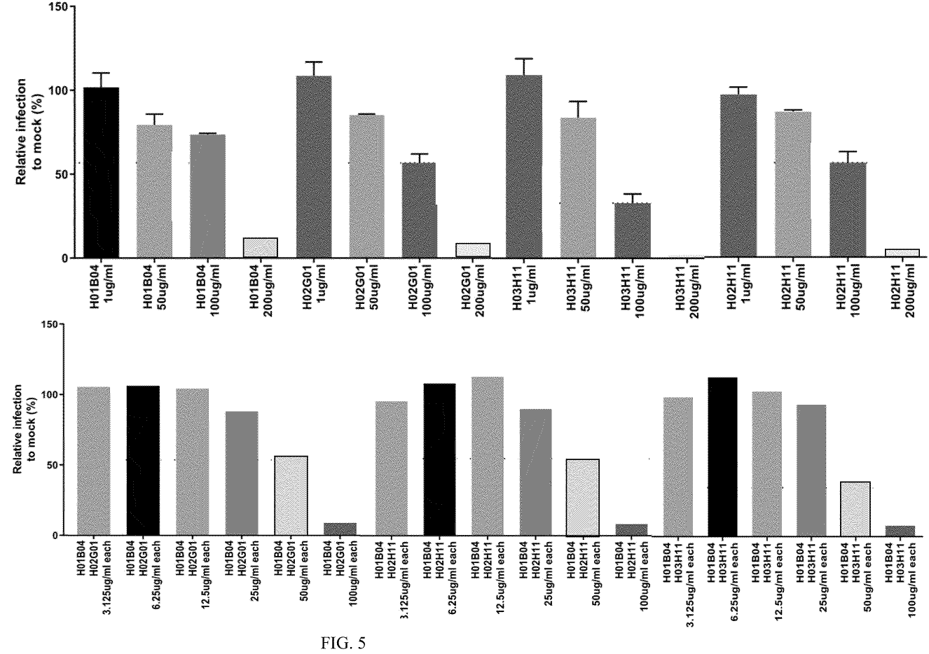

FIG. 5: Blockade of PRRS Type-1 virus BOR57 using VHH combinations comprising VHH02, VHH15, VHH16 and VHH20 The data show that individual VHH may be used in combination to block infection by PRRS Type-1 virus. A combination of VHH02 and VHH15, VHH02 and VHH16, as well as VHH02 and VHH20 showed effective blockade of productive virus infection over a concentration range of between 3 µg/mL to 100 µg/mL each of the VHH pair. The data demonstrate the potential for combinations of VHH to be used to block infection by PRRS virus. Data are shown relative to an infection in the absence of the test article compared to a mock control.

FIG. 6: X-ray crystallographic structure of the VHH14 (02D01) interaction with porcine CD163 SRCR5 domain The left-hand panel represents the refined structure of the VHH14:SRCR5 complex. On the left side is CD163:SRCR5 domain consisting of one long β sheet flanked by 2 shorter β sheets in a curved anti-parallel manner towards the N terminus, followed by a single α-helix. To the right is the core structure of VHH14 (02D01), composed of seven anti-parallel β sheets and three short α-helices positioned around the core. Both C- and N-terminal are denoted for each protein.

The panel on the right is an example of electron density (2 m|Fo|-D|Fc|) contoured at the 1 sigma level. The protein chain and individual amino acids are drawn as lines. The data show clear interactions between the Tyrosine (Y59) and Aspartic acid (D62) residues within the CDR2 domain of VHH14 with a pair of Leucine residues (Leu526, Leu 527) within the porcine SRCR5 domain. An additional interaction between Leucine 104 of VHH14 (CDR3) and a pair of amino acids in SRCR5 Serine 507 (S507) and Glutamic acid 509 (E509) is also seen. The crystal structure was refined to 2.0-2.3A. Leu (L) 52 in this Figure corresponds to Leu (L) 527 in the full length CD163 molecule shown in SEQ ID NO: 116. Leu (L) 51 in this Figure corresponds to Leu (L) 526 in the full length CD163 molecule shown in SEQ ID NO: 116. Ser (S) 32 in this Figure corresponds to Ser (S) 507 in the full length CD163 molecule shown in SEQ ID NO: 116. Glu (E) 34 in this Figure corresponds to Glu (E) 509 in the full length CD163 molecule shown in SEQ ID NO: 116. Asp (D) 62, Tyr (Y) 59 and Leu (L) 104 of VHH 02D01 are shown in SEQ ID NO:17′ or 99 (Table 3).

### EXAMPLES

### Example 1 Immunisation, Library Generation, Screening and Clone Selection Materials and Methods

### Immunization

Single domain antibodies were obtained from llamas immunized with recombinant protein. Llamas were injected with porcine CD163 Fc fusion antigen preparations formulated in Incomplete Freund’s Adjuvant (pCD163-SRCR1-9-huFc and pCD163-SRCR4-7-huFc). Animals were immunized with six subcutaneous injections (two injections with 100 µg/dose followed by four injections with 50 µg/dose) at weekly intervals. One week after the last boost, sera were collected to define antibody titers against pCD163-SRCR1-9-huFc and pCD163-SRCR4-7-huFc by ELISA.

In this ELISA, 96-well plates (Maxisorp; Nunc) were coated with the recombinant proteins. After blocking and adding diluted sera samples, the presence of anti pCD163 antibodies was demonstrated by using mouse anti-Camelid IgG⅔ (EMD millipore; Cat. nr. MAC131) followed by an anti-mouse immunoglobulin peroxidase conjugate (JIR, Cat. nr. 715-035-150).

### Library Construction

RNA was extracted from PBMC of 3 immunized llamas (400 ml each). 40 µg of RNA was used for cDNA synthesis using random primers. The cDNA was used in a primary PCR amplification using non-tagged primers annealing at the Leader sequence and Hinge CH1 regions, followed by a secondary PCR amplification introducing restriction endonuclease sites for cloning of VHH genes in pDCL1 phagemid vector. The libraries were electroporated into TG1 E. coli cells and bacterial glycerol stock of the immune libraries were stored at - 80° C. (FL1158 and FL1159)

### Selections

Phage production from the llama VHH library pool were used in two consecutive rounds of phage display selection using pCD163 recombinant protein or porcine pulmonary alveolar macrophages (pPAM). Selection rounds on recombinant proteins were performed using 10 µg/ml of pCD163-SRCR1-9-huFc or pCD163-SRCR4-7-huFc pH7.4 (PBS buffer) with washing of non-specific phage, followed by specific phage elution with trypsin (total elution). Selection rounds on pPAMs were performed using 5E106 cells at pH7.4 (PBS buffer) with washing of non-specific phage, followed by specific phage elution with trypsin (total elution). Serial dilutions of the eluted phages were performed and used to infect exponentially growing TG1. Infected TG1 was plated on LBCarb100Glu2% plates and enrichment values calculated over the background (without antigen for selection).

### ELISA Screening

Individual clones from the second round of selection conditions outputs were picked into 96-well Master Plates and tested as Periplasmic Extract (P.E.) for binding to pCD163-SRCR4-7-huFc or pCD163-SRCR5-6-huFc proteins at pH7.0 via binding ELISA. For P.E. binding ELISA, MaxiSorp™ high protein-binding capacity 96 well ELISA plates, were coated with 1 µg/ml of pCD163-SRCR4-7-huFc protein, diluted in PBS, overnight at 4° C. The next day, plates were washed 3X with PBS Tween 0.05% (pH 7.4) and blocked for 1 hour at room temperature with 250 µl/well of 4% Marvel/CPA or 4% Marvel/PBS. After blocking, plates were washed 3X with PBS Tween 0.05% (pH7.4) and incubated per well with 20 µl of P.E + 80 µl in 1% Marvel/PBS (pH7.4), for 1 hour at RT with shaking. Plates were washed 3X with PBS Tween 0.05% (pH7.4) and incubated with 100 µl of anti-c-Myc antibody (Roche; Cat. nr. 11667203001) followed by secondary antibody DAM-HRP (JIR; Cat. nr. 715-035-150) in 1% Marvel/PBS (pH7.4), for 1 hour at RT with shaking. Plates were washed 3X with PBS Tween 0.05% (pH7.4) and the substrate solution (TMB solution) was added to the plates. Reaction was stopped with H2SO4 and plates read in the plate reader at 450 nm.

### Screening on Cells (FACS)

Periplasmic Extract (P.E) from the selected clones were incubated with anti-c-myc antibody (Roche; Cat. nr. 11667203001), specific to the c-myc tag present in the soluble VHH, for 30 minutes with agitation at room temperature (RT). The mix (P.E + anti-c-myc antibody) was added to the pPAM or pPAMΔ5 domain (cells with deletion of SRCR domain 5) and incubated for 60 min at 4° C. with gentle shaking.

Cells were washed 3x with 150 µl/well of FACS Buffer and incubated with 50 µl/well of the secondary antibody GAM-APC for 30 min at 4° C. and protected from the light, with shaking.

Cells were washed 3x with 150 µl/well of FACS Buffer and resuspended in 75 µ/welll of FACS buffer to be measured in FACS machine (Attune™ NxT) in RL-1 channel (APC channel), and a total of 10000 cells were acquired per sample.

### Sequencing

The positive binders were sent to be sequenced. Clones were classified by families according to the different HCDR3 sequence.

### Results & Discussion

TABLE 5

ELISA Binding Data Summary

| Candidate | pCD163:SRCR4-7-Fc O.D. 450 | pCD163:SRCR1-9-Fc O.D. 450 | IgG O.D. 450 |
|---|---|---|---|
| VHH 001 (1B02) | 0.416 | 0.109 | 0.059 |
| VHH 002 (1B04) | 2.393 | 0.235 | 0.057 |
| VHH 008 (1H01) | 2.655 | 0.220 | 0.060 |
| VHH 015 (2G01) | 4.374 | 1.180 | 0.058 |
| VHH 016 (2H11) | 4.148 | 0.263 | 0.060 |
| VHH 018 (3E01) | 0.938 | 1.029 | 0.057 |
| VHH 019 (3E11) | 2.430 | 2.479 | 0.054 |
| VHH 020 (3H11) | 3.359 | 2.932 | 0.056 |
| VHH 023 (4E10) | 4.102 | 0.577 | 0.054 |
| VHH 011 (2B06) | 0.288 | 0.106 | 0.059 |
| VHH 012 (2C07) | 3.137 | 0.535 | 0.057 |
| VHH 014 (2D01) | 2.425 | 0.342 | 0.059 |
| VHH 017 (3D03) | 0.116 | 0.137 | 0.053 |

TABLE 6

Assessment of candidate binding to primary alveolar macrophage by flow cytometry

| Candidate | PAM CD163-WT (4C) | | PAM CD163-d5 | | PAM CD163-WT (RT) | |
|---|---|---|---|---|---|---|
| | % Binding | MFI | % Binding | MFI | % Binding | MFI |
| VHH 001 (1B02) | 0.97 | 742 | 2.15 | 1426 | 0.69 | 722 |
| VHH 002 (1B04) | 31.82 | 1984 | 1.74 | 1258 | 1.02 | 900 |
| VHH 008 (1H01) | 38.79 | 2371 | 2.70 | 1461 | 0.87 | 963 |
| VHH 015 (2G01) | 81.54 | 6216 | 1.60 | 1130 | 20.45 | 1662 |
| VHH 016 (2H11) | 60.99 | 3212 | 1.66 | 1360 | 1.59 | 927 |
| VHH 018 (3E01) | 80.13 | 5210 | 1.96 | 1404 | 1.74 | 903 |
| VHH 019 (3E11) | 93.79 | 12501 | 1.83 | 1533 | 59.45 | 3943 |
| VHH 020 (3H11) | 95.47 | 13957 | 1.98 | 1326 | 81.52 | 9121 |
| VHH 023 (4E10) | 8.75 | 1394 | 1.53 | 1258 | 0.56 | 843 |
| VHH 011 (2B06) | 74.08 | 4268 | 1.91 | 1613 | 3.32 | 983 |
| VHH 012 (2C07) | 97.91 | 26469 | 2.13 | 1583 | 91.46 | 17739 |
| VHH 014 (2D01) | 92.98 | 10791 | 1.73 | 1392 | 71.63 | 5404 |
| VHH 017 (3D03) | 95.29 | 13498 | 2.14 | 1451 | 37.21 | 2293 |

Following immunisation of llama with recombinant CD163, phage clones were selected by 2 rounds of phage panning. Phage candidates were confirmed by binding recombinant CD163 expression constructs (pCD163SRCR1-9-Fc or pCD163-SRCR4-7-Fc) by ELISA compared to control human IgG. Selective binding was demonstrated by several candidates to porcine CD163, as demonstrated in Table 5.

Clones were further selected for their ability to bind native membrane bound CD163 on isolated primary porcine alveolar macrophages, prepared from cells recovered from bronchoalveolar lavage from donor animals (Burkard C., et al PLOS Pathogens 2017). Candidate phage were demonstrated to bind to native membrane bound CD163 at both 4C, as well as at room temperature, when the CD163 receptor is more likely to become internalised by endocytic mechanisms.

All candidates were also assessed for selective binding to SRCR5 domain of CD163 by selection of candidates unable to bind porcine alveolar macrophages isolated from pigs with a deletion of this domain in the CD163 gene (Burkard C., et al PLOS Pathogens 2017). All selected candidates were unable to bind PAM’s isolated from these animals, so demonstrating preferential binding to the SRCR5 domain of porcine CD163.

Hence, the immunisation of llama with recombinant CD163 protein constructs resulted in the successful isolation of phage antibody candidates able to bind CD163 expressed on primary porcine alveolar macrophages, specifically targeting the SRCR5 domain, known to be essential for PRRS virus infection of these cells.

### Reference

Burkard C, Lillico SG, Reid E, Jackson B, Mileham AJ, Ait-Ali T, et al. (2017) Precision engineering for PRRSV resistance in pigs: Macrophages from genome edited pigs lacking CD163 SRCR5 domain are fully resistant to both PRRSV genotypes while maintaining biological function. PLoS Pathog 13(2): e1006206. doi:10.1371/journal.ppat.1006206

### Example 2 Affinity Determination of anti-CD163 VHH Antibodies to Porcine CD163

### Material and Methods

The affinity of binding for porcine CD163 by each of the identified VHH antibody candidates was determined by surface plasmon resonance.

### Expression and Purification of VHH Candidate Antibodies

The synthetic genes codifying to the VHH variable domains with FLAG and His tags were purchased and reconstituted according to manufacturer instructions. Each DNA construct was restriction enzyme digested, the insert was gel purified, and each variable domain insert was ligated with a mammalian expression vector pcDNA3.1. ExpiCHO-S cells were transfected with 23 lead panel sequences using 40 µg of total DNA plasmid constructs. A 25 mL total volume of cells was used for 8 days of protein production (32° C., 5% CO2). Produced VHH antibodies were captured from clarified supernatants using a HisTrap HP 5mL IMAC column (GE Healthcare, Cat. nr. 17-5248-02) on an ÄKTA Pure 25 FPLC system. Eluted antibody peak fractions were buffer exchanged to 1x PBS pH 7.4 and concentrated using 3 kDa MCO spin concentrators (Amicon, Cat. nr. UFC900324). Purified protein was analysed by analytical size exclusion chromatography (aSEC) and SDS-PAGE for the presence of correct chains.

### Affinity Determination

To assess the affinity of selected purified clones to pCD163, pCD163-SRCR1-9-huFc or pCD163-SRCR4-7-huFc proteins were coated by amine coupling on a CM5 sensor ship (GE Healthcare). Surface plasmon resonance (SPR) (Biacore 3000, GE Healthcare) was used to determine the binding kinetics of selected single domain antibodies at pH7.4. Approximately 2000 RU of pCD163-SRCR1-9-huFc or pCD163-SRCR4-7-huFc, proteins at 20 or 30 µg/ml in Acetate buffer pH 5.0 or pH 5.5 were immobilized onto a CM5 chip using the standard amine coupling procedure. QC of the immobilization was done using a commercial antibody anti-huFc (JIR, Cat. nr. 109-005-098) at the concentration of 30.0 µg/ml.

1x HBS-EP pH 7.4, were utilized as a running buffer during binding kinetic measurements. Purified VHH were injected at 3-fold dilution (300 nM; 100 nM; 33 nM; 11 nM; 3.7 nM; 1.2 nM; 0.4 nM; 0 nM), for 120 s, at a flow rate of 30 µl/min. RU levels were restored to base levels after regeneration with 10 µl of 10 mM NaOH / 1 M NaCl and 10 µl of 10 mM Glycine pH 1.5 between samples. Fitting 1:1 binding with mass transfer was applied to the set sample curves using the simultaneous fitting option of the BIAevaluation software to calculate the kinetic constants of the antibody-antigen interactions including association rate (ka), dissociation rate (kd) and affinity (KD). Curves were removed from the fitting after visual examination of the residuals and considering the value of Chi2: a minimum of 4 curves were considered for the simultaneous fitting.

### Results & Discussion

TABLE 7

Affinity determination of VHH for Porcine CD163

| Candidate | pCD163:SRCR4-7-Fc | | | pCD163:SRCR1-9-Fc | | |
|---|---|---|---|---|---|---|
| | ka(1/Ms) | kd (1/s) | KD (nM) | ka(1/Ms) | kd (1/s) | KD (nM) |
| VHH 001 (1B02) | 2.46E+06 | 2.27E-02 | 9.26 | 2.52E+06 | 1.78E-02 | 7.08 |
| VHH 002 (1B04) | 7.87E+05 | 1.49E-02 | 19.00 | 5.39E+05 | 1.08E-02 | 20.00 |
| VHH 008 (1H01) | 1.56E+05 | 5.42E-03 | 34.70 | N/D | N/D | N/D |
| VHH 015 (2G01) | 5.51E+05 | 4.42E-03 | 8.01 | 3.96E+05 | 5.83E-03 | 14.70 |
| VHH 016 (2H11) | 2.24E+05 | 6.74E-03 | 30.00 | 2.19E+05 | 9.72E-03 | 44.30 |
| VHH 018 (3E01) | 2.88E+05 | 1.21E-02 | 42.00 | 8.14E+05 | 1.34E-02 | 16.50 |
| VHH 019 (3E11) | 4.87E+05 | 1.03E-02 | 21.10 | 1.16E+06 | 1.02E-02 | 8.77 |
| VHH 020 (3H11) | 4.62E+05 | 8.43E-03 | 18.20 | 9.45E+05 | 8.65E-03 | 9.15 |
| VHH 023 (4E10) | 4.96E+06 | 7.55E-03 | 1.52 | 2.99E+06 | 9.27E-03 | 3.10 |
| VHH 011 (2B06) | 4.37E+05 | 1.51E-02 | 34.50 | 1.40E+05 | 1.00E-02 | 71.40 |
| VHH 012 (2C07) | 2.83E+06 | 4.26E-03 | 1.51 | 5.97E+06 | 6.17E-03 | 1.03 |
| VHH 014 (2D01) | 1.79E+06 | 1.11E-02 | 6.18 | 1.10E+06 | 1.33E-02 | 12.00 |
| VHH 017 (3D03) | 1.53E+05 | 3.45E-03 | 22.60 | 1.10E+06 | 8.30E-02 | 75.20 |

Phage candidates were expressed and purified as VHH, as described above. Characterisation of each of the VHH binding to either the full length, or truncated form of CD163 was performed as described above by surface plasmon resonance. Both the association and dissociation rates were determined for each antibody and an affinity determination calculated. As can be seen in Table 7, KD values for antibody candidates ranged between 1 and 75 nM. The relative affinity to either the full-length, or the truncated pCD163-SRCR4-7 constructs was broadly similar.

### Example 3 Inhibition of Porcine Respiratory and Reproductive (PRRS) Virus Infection of Primary Porcine Alveolar Macrophage Cells by Candidate CD163-specific Candidate Antibodies

### Materials and Methods

### PRRS Virus Infection Protocol

Reagents

VHH candidate antibodies were aliquoted at a concentration of 1 mg/mL

Control Antibodies:
Primary antibody: Anti-PRRS 1AC7, Ingenasa
Secondary antibody: Goat anti-mouse IgG (H+L) Alexa Fluor Plus 488, ThermoFisher, A32723

Culture Medium:

Complete RPMI (10% FBS or 80% (high) porcine serum, Ultraglutamine, Pen/Strep)

PAM Isolation: Porcine Alveolar Macrophage Isolation was conducted as described in Burkard et al PLoS Pathogens 2017.

Virus Isolates:

Type 1 Virus: BOR57 isolate (Roslin Institute, Edinburgh, UK)

Type 2 Virus: MN184 a US strain (Han et al 2006)

### Infection Protocol

Day 1 - Seed cells

Porcine alveolar macrophage cells were seeded at 20 million per plate in complete RPMI in 48 well plate and left in CO₂ incubator overnight

Day 2 - VHH Treatment & Infection Challenge
1. Pre-Treatment (30 minutes before infection)
   a. Aspirate medium off cells
   b. Add 100 µL culture medium to untreated uninfected and untreated infected controls
   c. Add 20 µL PBS in 100 µL culture medium to mock treated infected controls
   d. Add appropriate amount of VHH stock in 100 µL culture medium to treated and infected samples
   e. Return plate to CO₂ incubator for 30 minutes
2. Thaw virus stock and sonicate for 15 seconds before use
3. Infection Challenge (2 hours)
   a. Remove culture medium from cells and reserve VHH-containing culture medium for overnight incubation step
   b. Add 100 µL culture medium to untreated uninfected controls
   c. Add 10 µL virus in 100 µL culture medium to untreated infected controls
   d. Add 10 µL virus plus 20 µL PBS in 100 µL culture medium in mock treated infected controls
   e. Add appropriate amount of VHH stock and 10 µL virus in 100 µL culture medium to treated and infected samples
   f. Gently agitate plate and return to CO₂ incubator
   g. Gently agitate plate every 15 minutes for 2 hours
4. Overnight Incubation (15 hours)
   a. Aspirate medium off cells
   b. Add 100 µL culture medium to untreated uninfected controls and untreated infected controls
   c. Add 20 µL PBS in 100 µL culture medium to mock treated infected controls
   d. Add reserved VHH containing culture medium from the pre-treatment step to appropriate samples
   e. Return plate to CO₂ incubator for 15 hours

Day 3 - In-well Fix and Stain Protocol
5. Aspirate medium from cells
6. Fix cells in 4% formaldehyde/PBS++ (with calcium & magnesium) solution at RT for 30 minutes
7. Wash once with PBS++
8. Permeabilise with Triton-X (1% in PBS++) at RT for 5 minutes
9. Wash once with PBS++ or blocking solution (PBS++/5% FBS)
10. Block with blocking solution (PBS++/5% FBS) at RT for 20 minutes
11. Add primary antibody Anti-PRRS 1AC7 at 1:5000 to all wells except for unstained controls and secondary antibody only controls
12. Incubate at RT for 1 hour
13. Wash three time with PBS++
14. Add secondary antibody Goat anti-mouse IgG (H+L) Alexa Fluor Plus 488 at 1:5000 to all wells except for unstained controls
15. Incubate at RT for 45 minutes
16. Wash three time with PBS++
17. Add 300 µL PBS++
18. Scrape cells using a wide bore p200 pipette tip, then scrape around the edges of the well with a normal p200 tip and then use a p1000 to wash the well surface 3x and collect the cells for transfer into a FACs tube .
19. Measurements were conducted on Fortessa x20 (Voltages: FSC=418, SSC=308 & B530-30=386)

### Results and Discussion

The ability of individual VHH to block infection of PAM host cells by PRRS virus family members is described below. The assay, as described above was used to measure the extent of virus infection as quantified by the ability to propagate virus, as measured by FACS following a 17 hour infection cycle. The data presented are shown in FIG. 1 and FIG. 2 as well as summarised in Table 8 below.

TABLE 8

Blockade of PRRS Virus Infection

| Virus | (µg/mL) | | (µg/mL) |
|---|---|---|---|
| Candidate VHH | 10% FBS | 80% Serum | 10% FBS |
| | IC₅₀ | IC₅₀ | IC₅₀ |
| VHH 001 (1B02) | 147 | 249 | 202 |
| VHH 002 (1B04) | 130 | 226 | nd |
| VHH 008 (1H01) | 127 | 248 | 209 |
| VHH 015 (2G01) | 107 | 211 | nd |
| VHH 016 (2H11) | 103 | 179 | 175 |
| VHH 018 (3E01) | 110 | 223 | 180 |
| VHH 019 (3E11) | 85.52 | 160 | 108 |
| VHH 020 (3H11) | 79 | 348 | 153 |
| VHH 023 (4E10) | 163 | 287 | 242 |
| VHH 011 (2B06) | nd | nd | 279 |
| VHH 012 (2C07) | nd | nd | 257 |
| VHH 014 (2D01) | nd | nd | 242 |
| VHH 017 (3D03) | nd | nd | 208 |

n.d. - not determined

The data clearly show VHH able to inhibit productive infection of porcine alveolar macrophage cells by both PRRS Type 1 (see also FIG. 1) and Type 2 (see also FIG. 2) isotypes. The VHH showing activity in the infection assays could be divided into those that were effective against both Type 1 and Type 2 virus infection (VHH’s 001, 002, 008, 015, 016, 018, 019, 020, and 023), and those which did not show any inhibitory activity against Type 1 PRRS virus infection, but which showed selective inhibitory activity against Type 2 PRRS virus infection (VHHs 011, 012, 014, 017). The data clearly show VHH able to inhibit productive infection of porcine alveolar macrophage cells by both PRRS Type 1 & 2 isotypes.

Some VHH only showed inhibitory activity in the Type 2 virus infection (VHH’s 011, 012, 014, 017) across a concentration range up to 300 µg/mL (FIG. 2). These candidate VHH showed specificity for PRRS Type 2 virus and did not show any inhibitory activity in a Type 1 virus infection assay across a similar dose response concentration range up to 100 µg/mL (FIG. 3).

### References

Burkard C., et al Precision engineering for PRRSV resistance in pigs: Macrophages from genome edited pigs lacking CD163 SRCR5 domain are fully resistant to both PRRSV genotypes while maintaining biological function PLoS Pathogens, 13(2) 2017: e1006206

Han J., Y. Wang, K.S.Faaberg. Complete genome analysis of RFLP 184 isolates of porcine reproductive and respiratory syndrome virus Virus Research, 122 (2006), pp. 175.

### Example 4 Inhibition of Porcine Respiratory and Reproductive (PRRS) Virus Infection of Primary Porcine Alveolar Macrophage Cells by Combinations of Candidate CD163-Specific Candidate Antibodies

### Materials and Methods

### PRRS Virus Infection Protocol

Reagents

VHH candidate antibodies were aliquoted at a concentration of 1 mg/mL

Control Antibodies:

Primary antibody: Anti-PRRS 1AC7, Ingenasa

Secondary antibody: Goat anti-mouse IgG (H+L) Alexa Fluor Plus 488, ThermoFisher, A32723

Culture Medium:

Complete RPMI (10% FBS or 80% high porcine serum, Ultraglutamine, Pen/Strep)

PAM Isolation: Porcine Alveolar Macrophage Isolation was conducted as described in Burkard et al PLoS Pathogens 2017.

Virus Isolates:

Type 1 Virus: BOR57 isolate (Roslin Institute, Edinburgh, UK)

Type 2 Virus: MN184 a US strain (Han et al 2006)

Infection Protocol

Day 1 - Seed cells

Porcine alveolar macrophage cells were seeded at 20 million per plate in complete RPMI in 48 well plate and left in CO₂ incubator overnight

Day 2 - VHH Treatment & Infection Challenge
1. Pre-Treatment (30 minutes before infection)
   a. Aspirate medium off cells
   b. Add 100 µL culture medium to untreated uninfected and untreated infected controls
   c. Add 20 µLY PBS in YL culture medium to mock treated infected controls
   d. Add appropriate amount of VHH stock YL culture medium to treated and infected samples
   e. Return plate to CO₂ incubator for 30 minutes
2. Thaw virus stock and sonicate for 15 seconds before use
3. Infection Challenge (2 hours)
   a. Remove culture medium from cells and reserve VHH-containing culture medium for overnight incubation step
   bYculture medium to untreated uninfected controls
   c. Add 10 µL virus in 100 µL culture medium to untreated infected controls
   d. Add 10 µL virus plus 20 µL PBS in 100 µL culture medium in mock treated infected controls
   e. Add appropriate amount of individual VHH stock solution (or combinations of VHH) and 10 µL virus in 100 µL culture medium to treated and infected samples
   f. Gently agitate plate and return to CO₂ incubator
   g. Gently agitate plate every 15 minutes for 2 hours
4. Overnight Incubation (15 hours)
   a. Aspirate medium off cells
   b. Add 100 µL culture medium to untreated uninfected controls and untreated infected controls
   c. Add 20 µL PBS in 100 µL culture medium to mock treated infected controls
   d. Add reserved VHH containing culture medium from the pre-treatment step to appropriate samples
   e. Return plate to CO₂ incubator for 15 hours

Day 3 - In-well Fix and Stain Protocol
5. Aspirate medium from cells
6. Fix cells in 4% formaldehyde/PBS++ (with calcium & magnesium) solution at RT for 30 minutes
7. Wash once with PBS++
8. Permeabilise with Triton-X (1% in PBS++) at RT for 5 minutes
9. Wash once with PBS++ or blocking solution (PBS++/5% FBS)
10. Block with blocking solution (PBS++/5% FBS) at RT for 20 minutes
11. Add primary antibody Anti-PRRS 1AC7 at 1:5000 to all wells except for unstained controls and secondary antibody only controls
12. Incubate at RT for 1 hour
13. Wash three time with PBS++
14. Add secondary antibody Goat anti-mouse IgG (H+L) Alexa Fluor Plus 488 at 1:5000 to all wells except for unstained controls
15. Incubate at RT for 45 minutes
16. Wash three time with PBS++
17. Add 300 µL PBS++
18. Scrape cells using a wide bore p200 pipette tip, then scrape around the edges of the well with a normal p200 tip and then use a p1000 to wash the well surface 3x and collect the cells for transfer into a FACs tube
19. Measurements were conducted on Fortessa x20 (Voltages: FSC=418, SSC=308 & B530-30=386)

### Results and Discussion

The ability of individual VHH to block infection of PAM host cells by PRRS virus family members is described below. The assay, as described above was used to measure the extent of virus infection as quantified by the ability to propagate virus, as measured by FACS following a 17 hour infection cycle. The data presented are shown in FIG. 4 and FIG. 5.

Combinations of VHH 15, 16 and 20 were used in infection assays using the BOR57 PRRS Type-1 virus in the presence of medium containing 10% FBS. Specific combinations of 100 µg each of VHH 15 and VHH16, VHH15 and VHH20, VHH16 and VHH20, and a triple combination of 50 µg each of VHH15+VHH16+VHH20 showed potential to reduce infection potential of PRRS Type-1 virus to very low levels of infectivity (see FIG. 4).

The potential of different pairwise combinations of VHH was explored in infection assays using the BOR57 PRRS Type-1 virus in the presence of medium containing 10% FBS. VHH02 (01B04) was used as a partner with VHH15 (02G01), VHH16 (02H11) and VHH20 (03H11) to assess the potential to block infection. Combinations were tested at increasing concentrations of each partner VHH. The data demonstrate that combinations of VHH may be used to block PRRS Type-1 virus infection (see FIG. 5).

The data show that combinations of VHH are able to enhance inhibition of productive infection of porcine alveolar macrophage cells by PRRS Type 1 isotypes to a potentially greater extent than individual VHH candidates. The data suggests that it may be possible to combine individual VHH to enhance potential blockade of infection by PRRS virus family members.

### References

Burkard C., et al, supra; Han J., Y. Wang, K.S.Faaberg, supra.

### Example 5: Determination of anti-CD163 VHH Antibodies Binding to Porcine CD163 SRCR5

### Material and Methods

The X-ray crystallographic structure for porcine CD163 SRCR5 domain binding to VHH antibody candidate 2D01 (VHH14) was determined.

### Expression and Purification of VHH14 Antibody

The synthetic genes codifying to the VHH variable domains with FLAG and His tags were purchased and reconstituted according to manufacture instructions. Each DNA construct was restriction enzyme digested, the insert was gel purified, and each variable domain insert was ligated with a mammalian expression vector pcDNA3.1. ExpiCHO-S cells were transfected with 23 lead panel sequences using 40 µg of total DNA plasmid constructs. A 25 mL total volume of cells was used for 8 days of protein production (32° C., 5% CO2). Produced VHH antibodies were captured from clarified supernatants using a HisTrap HP 5mL IMAC column (GE Healthcare, Cat. nr. 17-5248-02) on an ÄKTA Pure 25 FPLC system. Eluted antibody peak fractions were buffer exchanged to 1x PBS pH 7.4 and concentrated using 3 kDa MCO spin concentrators (Amicon, Cat. nr. UFC900324). Purified protein was analysed by analytical size exclusion chromatography (aSEC) and SDS-PAGE for the presence of correct chains.

### Expression and Purification of Porcine SRCR5

The synthetic gene coding for porcine CD163 SRCR5 region with a ^x HIS tag was subcloned into a pTXBac1 (proprietary vector) prior to transformation into E.coli DH10Bac to produce a recombinant Bacmid. The recombinant Bacmid was used to transform Spodoptera frugiperda (Sf) cells to generate a P1 virus clones. Cells and medium samples from the P1 clones were checked routinely for protein expression to identify high expressing clones. High expressing clones were amplified by infection of Sf cells with the P1 virus stock to generate a P2 virus stock, subsequently used to express recombinant SRCR5 protein in Sf1 cells over a 72 hour period at a multiplicity of infection (MOI) ~1.

Production scale-up was done in a 10 L culture of Sf cells. Optimal expression conditions were used as described above. The culture supernatant was equilibrated with phosphate-buffered saline (PBS) pH7.5 prior to purification using a HIS tag on an IMAC by a standard protocol. Samples were washed with PBS pH7.5 and 0.1% Triton X-114 buffer. Protein was eluted with imidazole according to manufacturer’s instructions. The eluted samples were buffer exchanged with PBS pH7.5, prior to a further purification on cobalt-resin. Samples were eluted following washing (as described above) using an imidazole shift. The resultant eluted samples were buffer exchanged to 20 mM Tris-HCl, pH7.4, 150 mM NaCl.

TABLE 9

| | |
|---|---|
| Protein Construct | Sequence |
| Porcine SRCR5 (SEQ ID NO: 117) | MPRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCSGHHHHHH |
| VHH14 (02D01) (SEQ ID NO: 118) | QLQLVESGGGLVQPGGSLRLSCAASGRTFSTDTMAWFRQAPGKEREFIAGIGRSGGSIYYADAVKGRFTVSRDNAKNTVYLQMNSLKAEDTAVYYCAARQRIGLVVGALGYDYWGQGTQVTVSSDYKDDDDKGGGGSHHHHHH |

### Crystallisation & X-ray Structure Determination Studies

The CD163 SRCR5:VHH14 complex was prepared at a concentration of 11.84 mg/mL in PBS pH7.4 buffer. Plate-like crystals were grown in 0.2 M NaCl, 0.1 M phosphate/citrate pH4.5, 20% w/v PEG8000. Individual crystals were flash-frozen in liquid nitrogen after addition of cryo-solution containing: 0.14 M NaCl, 0.07 M phosphate/citrate buffer pH4.5, 13.9% PEG 8000 and 46% ethylene glycol.

Data was collected at 100 K at the BioMAX beamline of MAX IV in Sweden (I= 0.97625 Å). The beamline was equipped with an Eiger 16 M hybrid-pixel detector.

The structure was determined using Phaser software and two homologous proteins (PDB code: 5DA4 and 5JFB), determined to 2.4 Å and 2.0 Å respectively. It was possible to model 103 amino acids of the CD163 SRCR5 domain (in bold Table 9) and 122 amino acids for the VHH014 (02D01), residue 4 onwards marked in bold (Table 9).

### Results & Discussion

The structure of the CD163 SRCR5: VHH014 complex reveals a single monomeric SRCR5 domain (left side) interacting with a single monomeric VHH014 antibody (right side) as shown in FIG. 6. The electron density map reveals specific interactions between residues in VHH014 and identified amino acids in the CD163:SRCR5 domain. Particularly,

Leucine 526 and Leucine 527 within the porcine CD163 SRCR5 domain appear to form interactions with specific conserved residues, according to an XYAD/E/N motif in CDR2 of VHH14, a motif shared between all the VHH candidates identified. VHH14 (02D01) is interesting as it does not inhibit infection by PRRS Type-1 virus, but is able to reduce infection by PRRS Type-2 family virus. The di-leucine motif identified in porcine CD163 SRCR5 may represent a feature important for interaction of PRRS Type-2 virus with SRCR5 and productive infection of porcine alveolar macrophages.

1. A monoclonal antibody which binds to porcine CD163 for use in the treatment or prevention of PRRS virus infection in a pig.

2. The monoclonal antibody of claim 1, wherein said antibody has the ability to bind to the SRCR5 domain of porcine CD163.

3. The monoclonal antibody of claim 1 or claim 2, wherein said antibody has the ability to inhibit type 1 and/or type 2 PRRSV infection.

4. An antibody suitable for the use of any one of claims 1 to 3, comprising an antigen binding domain which binds to porcine CD163, said antigen binding domain comprising at least one heavy chain variable region which comprises three complementarity determining regions (CDRs), wherein said heavy chain variable region comprises a variable heavy (VH) CDR2 that comprises the amino acid sequence XYAD or XYAE or XYAN, in which X can be any amino acid, preferably Y, L, P, N, F, or R, more preferably Y, F, L, N, or R, or Y, P or L, most preferably Y.

5. An antibody suitable for the use of any one of claims 1 to 3, or the antibody of claim 4, comprising an antigen binding domain which binds to an epitope in the SRCR5 domain of porcine CD163 comprising or corresponding to amino acids L526 and L527 of porcine CD163 (SEQ ID NO:116).

6. The antibody of claim 5, wherein said antigen binding domain binds to an epitope in the SRCR5 domain of porcine CD163 comprising or corresponding to amino acids L526, L527 and S507, or L526, L527 and E509, or L526, L527, S507 and E509 of SEQ ID NO: 116.

7. An antibody suitable for the use of any one of claims 1 to 3, or the antibody of any one of claims 4 to 6, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYVMG (SEQ ID NO:2), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of GIAWSGRAPYADSVKG (SEQ ID NO:3), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GEGAIRWTTLDAYDY (SEQ ID NO:4), or a sequence substantially homologous thereto, wherein said substantially homologous sequence is a sequence containing 1, 2, 3 or 4 amino acid substitutions compared to the given CDR sequence.

8. The antibody of claim 7, wherein said heavy chain variable region comprises:
(i) a VH CDR1 of RYVMG (SEQ ID NO:2) or a sequence substantially homologous thereto, wherein said substantially homologous sequence is a sequence containing 1 or 2 altered amino acids compared with the given CDR sequence,
(ii) a VH CDR2 of X₁ I X₃ W S G R A P Y A D S V K G (SEQ ID NO:73) wherein X₁ or X₃ can be any amino acid, preferably X₁ is G or A and/or X₃ is A or S, and
(iii) a VH CDR3 of G E G A I X₆ W T T X₁₀ X₁₁ A Y X₁₄ Y (SEQ ID NO:75) wherein X₆, X₁₀X₁₁ and X₁₄ can be any amino acid, preferably X₆ is R or K or L, X₁₀ is L or P, X₁₁ is D or G, and/or X₁₄ is D or N.

9. The antibody of claim 7 or claim 8, wherein said heavy chain variable region comprises:
(i) a variable heavy (VH) CDR1 that comprises the amino acid sequence of RYVMG (SEQ ID NO:10),
(ii) a variable heavy (VH) CDR2 that comprises the amino acid sequence of AISWSGRAPYADSVKG (SEQ ID NO:11), and
(iii) a variable heavy (VH) CDR3 that comprises the amino acid sequence of GEGAIKWTTLDAYDY (SEQ ID NO:12).

10. The antibody of claim 7 or claim 8, wherein said heavy chain variable region comprises:
(i) a variable heavy (VH) CDR1 that comprises the amino acid sequence of RYVMG (SEQ ID NO:2),
(ii) a variable heavy (VH) CDR2 that comprises the amino acid sequence of GIAWSGRAPYADSVKG (SEQ ID NO:3), and
(iii) a variable heavy (VH) CDR3 that comprises the amino acid sequence of GEGAIRWTTLDAYDY (SEQ ID NO:4).

11. The antibody of claim 7 or claim 8, wherein said heavy chain variable region comprises:
(i) a variable heavy (VH) CDR1 that comprises the amino acid sequence of RYVMG (SEQ ID NO:18),
(ii) a variable heavy (VH) CDR2 that comprises the amino acid sequence of GIAWSGRAPYADSVKG (SEQ ID NO:19), and
(iii) a variable heavy (VH) CDR3 that comprises the amino acid sequence of GEGAILWTTPGAYNY (SEQ ID NO:20).

12. An antibody suitable for the use of any one of claims 1 to 3, or the antibody of any one of claims 4 to 6, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYSMG (SEQ ID NO:26), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AHRWSGSAYYAEHADSVEG (SEQ ID NO:27), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GVGSAAQYRY (SEQ ID NO:28), or a sequence substantially homologous thereto, wherein said substantially homologous sequence is a sequence containing 1, 2, 3 or 4 amino acid substitutions compared to the given CDR sequence.

13. The antibody of claim 12, wherein said heavy chain variable region comprises:
(i) a VH CDR1 of X₁ X₂ S M G (SEQ ID NO:77), wherein X₁ or X₂ can be any amino acid, preferably X₁ is T or P and/or X₂ is Y or G,
(ii) a VH CDR2 of AH R W S G SAY Y A X₁₂ X₁₃ A D S V E G (SEQ ID NO:79), wherein X₁₂ or X₁₃ can be any amino acid, preferably X₁₂ is E or D and/or X₁₃ is H or Y,
(iii) a VH CDR3 of G V G S X₅ A Q Y X₉ Y (SEQ ID NO:81), wherein X₅ and X₉ can be any amino acid, preferably X₅ is A or E and/or X₉ is R or T.

14. The antibody of claim 12 or claim 13, wherein said heavy chain variable region comprises:
(i) a variable heavy (VH) CDR1 that comprises the amino acid sequence of TYSMG (SEQ ID NO:26),
(ii) a variable heavy (VH) CDR2 that comprises the amino acid sequence of AHRWSGSAYYAEHADSVEG (SEQ ID NO:27), and
(iii) a variable heavy (VH) CDR3 that comprises the amino acid sequence of GVGSAAQYRY (SEQ ID NO:28).

15. The antibody of claim 12 or claim 13, wherein said heavy chain variable region comprises:
(i) a variable heavy (VH) CDR1 that comprises the amino acid sequence of PGSMG (SEQ ID NO:34),
(ii) a variable heavy (VH) CDR2 that comprises the amino acid sequence of AHRWSGSAYYADYADSVEG (SEQ ID NO:35), and
(iii) a variable heavy (VH) CDR3 that comprises the amino acid sequence of GVGSAAQYTY (SEQ ID NO:36).

16. The antibody of claim 12 or claim 13, wherein said heavy chain variable region comprises:
(i) a variable heavy (VH) CDR1 that comprises the amino acid sequence of TYSMG (SEQ ID NO:42),
(ii) a variable heavy (VH) CDR2 that comprises the amino acid sequence of AHRWSGSAYYAEHADSVEG (SEQ ID NO:43), and
(iii) a variable heavy (VH) CDR3 that comprises the amino acid sequence of GVGSEAQYRY (SEQ ID NO:44).

17. An antibody suitable for the use of any one of claims 1 to 3, or the antibody of any one of claims 4 to 6, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of SYSMG (SEQ ID NO:50), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AITWNGYITNYADSVKG (SEQ ID NO:51), or a sequence substantially homologous thereto, wherein said substantially homologous sequence is a sequence containing 1, 2, 3 or 4 amino acid substitutions compared to the given CDR sequence, and,
(iii) a variable heavy (VH) CDR3 that comprises the amino acid sequence of TTFSTTSPISRTYNY (SEQ ID NO:52), or a sequence substantially homologous thereto, wherein said substantially homologous sequence is a sequence containing 1, 2, 3 or 4 amino acid substitutions compared to the given CDR sequence.

18. An antibody suitable for the use of any one of claims 1 to 3, or the antibody of any one of claims 4 to 6, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TYAMG (SEQ ID NO:58), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of IISFGGTFYADSVKG (SEQ ID NO:59), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GRTLSKRADSYAS (SEQ ID NO:60), or a sequence substantially homologous thereto, wherein said substantially homologous sequence is a sequence containing 1, 2, 3 or 4 amino acid substitutions compared to the given CDR sequence.

19. An antibody suitable for the use of any one of claims 1 to 3, or the antibody of any one of claims 4 to 6, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of MYAMS (SEQ ID NO:66), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AINTSGRYSRYADSVKG (SEQ ID NO:67), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of TDKGNWALAMSYDY (SEQ ID NO:68), or a sequence substantially homologous thereto, wherein said substantially homologous sequence is a sequence containing 1, 2, 3 or 4 amino acid substitutions compared to the given CDR sequence.

20. An antibody suitable for the use of any one of claims 1 to 3, or the antibody of any one of claims 4 to 6, wherein said antibody can specifically inhibit type 2 PRRSV infection.

21. The antibody of claim 20, wherein said antibody can specifically inhibit type 2 PRRSV infection, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of VYGTG (SEQ ID NO:84), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of GISGTTGSTLYADSVKG (SEQ ID NO:85), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of GGRVYITTSSWAY (SEQ ID NO:86), or a sequence substantially homologous thereto, wherein said substantially homologous sequence is a sequence containing 1, 2, 3 or 4 amino acid substitutions compared to the given CDR sequence.

22. The antibody of claim 20, wherein said antibody can specifically inhibit type 2 PRRSV infection, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of RYAMG (SEQ ID NO:92), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of AIAWSTGSTYYANSVKG (SEQ ID NO:93), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of ETRYCSGFGCLDPRTYGS (SEQ ID NO:94), or a sequence substantially homologous thereto, wherein said substantially homologous sequence is a sequence containing 1, 2, 3 or 4 amino acid substitutions compared to the given CDR sequence.

23. The antibody of claim 20, wherein said antibody can specifically inhibit type 2 PRRSV infection, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of TDTMA (SEQ ID NO: 100), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of GIGRSGGSIYYADAVKG (SEQ ID NO:101), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of RQRIGLVVGALGYDY (SEQ ID NO:102), or a sequence substantially homologous thereto, wherein said substantially homologous sequence is a sequence containing 1, 2, 3 or 4 amino acid substitutions compared to the given CDR sequence.

24. The antibody of claim 20, wherein said antibody can specifically inhibit type 2 PRRSV infection, comprising an antigen binding domain which binds to porcine CD163, said antigen binding domain comprising at least one heavy chain variable region which comprises three complementarity determining regions (CDRs), wherein said heavy chain variable region comprises:
(i) a variable heavy (VH) CDR1 that comprises the amino acid sequence of DYTIG (SEQ ID NO:108), or a sequence substantially homologous thereto, wherein said substantially homologous sequence is a sequence containing 1 or 2 amino acid substitutions compared to the given CDR sequence,
(ii) a variable heavy (VH) CDR2 that comprises the amino acid sequence of CINSITSNTYYADSVKG (SEQ ID NO:109), or a sequence substantially homologous thereto, wherein said substantially homologous sequence is a sequence containing 1, 2, 3 or 4 amino acid substitutions compared to the given CDR sequence, and
(iii) a variable heavy (VH) CDR3 that comprises the amino acid sequence of DSGLFSGSSCLKYRAMRFGS (SEQ ID NO:110), or a sequence substantially homologous thereto, wherein said substantially homologous sequence is a sequence containing 1, 2, 3 or 4 amino acid substitutions compared to the given CDR sequence.

25. An antibody suitable for use of any one of claims 1 to 3, or the antibody of any one of claims 4 to 24, wherein the antibody is a single domain antibody.

26. An antibody suitable for use of any one of claims 1 to 3, or the antibody of any one of claims 4 to 19 or claim 25, wherein said antibody can inhibit type 1 and type 2 PRRSV infection, preferably wherein said antibody can inhibit the ability of type 2 PRRSV to infect host cells by at least 50%, and/or can inhibit the ability of type 1 PRRSV to infect host cells by at least 50%, at least 80% or at least 90%.

27. An antibody suitable for use of any one of claims 1 to 3, or the antibody of any one of claims 4 to 6, or claims 20 to 25, wherein said antibody can specifically inhibit type 2 PRRSV infection, preferably wherein said antibody can inhibit the ability of type 2 PRRSV to infect host cells by at least 40%, more preferably wherein said antibody does not significantly inhibit the ability of type 1 PRRSV to infect host cells.

28. An antibody which binds to the same epitope of porcine CD163 as the antibody of any one of claims 7 to 24.

29. A combination of two or more antibodies of any one of claims 7 to 24, or a combination of an antibody of any one of claims 7 to 19 with an antibody of any one of claims 21 to 24.

30. One or more nucleic acid molecules comprising nucleotide sequences that encode the antibody of any one of claims 4 to 28; or
one or more expression vectors comprising such nucleic acid molecules; or
one or more host cells comprising said expression vectors or nucleic acid molecules, or
expressing an antibody of any one of claims 4 to 28.

31. A method of producing an antibody of any one of claims 4 to 28, said method comprising the steps of (i) culturing a host cell comprising one or more of the expression vectors or one or more of the nucleic acid sequences as defined in claim 30 under conditions suitable for the expression of the encoded antibody; and optionally (ii) isolating or obtaining the expressed antibody from the host cell or from the growth medium/supernatant.

32. A composition, preferably a pharmaceutically acceptable composition, comprising an antibody of any one of claims 4 to 28, or the combination of antibodies of claim 29, or one or more nucleic acid molecules or expression vectors of claim 30.

33. The antibody of any one of claims 4 to 28, or the combination of antibodies of claim 29, or the one or more nucleic acid molecules or expression vectors of claim 30, for use in therapy, preferably for use in the treatment or prevention of PRRS virus infection in a pig.

34. Use of the antibody of any one of claims 4 to 28, or the combination of antibodies of claim 29, or the one or more nucleic acid molecules or expression vectors of claim 30, in the manufacture of a medicament or composition for use in the treatment or prevention of PRRS virus infection in a pig.

35. A method of treatment or prevention of PRRS virus infection in a pig, wherein said method comprises the step of administering to a subject in need thereof a therapeutically effective amount of the antibody of any one of claims 4 to 28, or the combination of antibodies of claim 29, or the one or more nucleic acid molecules or expression vectors of claim 30.

36. The molecules for use of claim 33, the use of claim 34, or the method of claim 35, in the treatment or prevention of type 1 and/or type 2 PRRSV infection.


SEQUENCE LISTING (118 sequences)

SEQ ID NO: 1 - protein, 123 aa, Llama
QVQLQESGGGLVQVGGSLRLSCVASGRAPSRYVMGWFRQAPGQEREFVAGIAWSGRAPYADSVKGRSTISRDNAKNTVYLQMNSLKPEDTGVYYCAGGEGAIRWTTLDAYDYWGQGTQVTVSS

SEQ ID NO: 2 - protein, 5 aa, Llama
RYVMG

SEQ ID NO: 3 - protein, 16 aa, Llama
GIAWSGRAPYADSVKG

SEQ ID NO: 4 - protein, 15 aa, Llama
GEGAIRWTTLDAYDY

SEQ ID NO: 5 - protein, 30 aa, Llama
QVQLQESGGGLVQVGGSLRLSCVASGRAPS

SEQ ID NO: 6 - protein, 14 aa, Llama
WFRQAPGQEREFVA

SEQ ID NO: 7 - protein, 32 aa, Llama
RSTISRDNAKNTVYLQMNSLKPEDTGVYYCAG

SEQ ID NO: 8 - protein, 11 aa, Llama
WGQGTQVTVSS

SEQ ID NO: 9 - protein, 123 aa, Llama
QVQLQESGGGLVQVGSSLRLSCVTSGRTPSRYVMGWFRQAPGQEREFVAAISWSGRAPYADSVKGRFTISRDNAKNTVYLQMNSLKPEDTGVYYCAGGEGAIKWTTLDAYDYWGQGTQVTVSS

SEQ ID NO: 10 - protein, 5 aa, Llama
RYVMG

SEQ ID NO: 11 - protein, 16 aa, Llama
AISWSGRAPYADSVKG

SEQ ID NO: 12 - protein, 15 aa, Llama
GEGAIKWTTLDAYDY

SEQ ID NO: 13 - protein, 30 aa, Llama
QVQLQESGGGLVQVGSSLRLSCVTSGRTPS

SEQ ID NO: 14 - protein, 14 aa, Llama
WFRQAPGQEREFVA

SEQ ID NO: 15 - protein, 32 aa, Llama
RFTISRDNAKNTVYLQMNSLKPEDTGVYYCAG

SEQ ID NO: 16 - protein, 11 aa, Llama
WGQGTQVTVSS

SEQ ID NO: 17 - protein, 123 aa, Llama
QVQLQESGGGLVQVGGSLRLSCVASGRTPSRYVMGWFRQAPGQEREFVAGIAWSGRAPYADSVKGRFVISRDSAKNTVYLQMNSLKSEDTGVYYCAGGEGAILWTTPGAYNYWGQGTQVTVSS

SEQ ID NO: 18 - protein, 5 aa, Llama
RYVMG

SEQ ID NO: 19 - protein, 16 aa, Llama
GIAWSGRAPYADSVKG

SEQ ID NO: 20 - protein, 15 aa, Llama
GEGAILWTTPGAYNY

SEQ ID NO: 21 - protein, 30 aa, Llama
QVQLQESGGGLVQVGGSLRLSCVASGRTPS

SEQ ID NO: 22 - protein, 14 aa, Llama
WFRQAPGQEREFVA

SEQ ID NO: 23 - protein, 32 aa, Llama
RFVISRDSAKNTVYLQMNSLKSEDTGVYYCAG

SEQ ID NO: 24 - protein, 11 aa, Llama
WGQGTQVTVSS

SEQ ID NO: 25 - protein, 121 aa, Llama
QVQLVESGGGLVQAGGSLRLSCAASGLTFVTYSMGWFRQAPGKEREFVAAHRWSGSAYYAEHADSVEGRFTISRDYAKNMLYLQMNSLKHEDTAVYYCAAGVGSAAQYRYWGRGTQVTVSS

SEQ ID NO: 26 - protein, 5 aa, Llama
TYSMG

SEQ ID NO: 27 - protein, 19 aa, Llama
AHRWSGSAYYAEHADSVEG

SEQ ID NO: 28 - protein, 10 aa, Llama
GVGSAAQYRY

SEQ ID NO: 29 - protein, 30 aa, Llama
QVQLVESGGGLVQAGGSLRLSCAASGLTFV

SEQ ID NO: 30 - protein, 14 aa, Llama
WFRQAPGKEREFVA

SEQ ID NO: 31 - protein, 32 aa, Llama
RFTISRDYAKNMLYLQMNSLKHEDTAVYYCAA

SEQ ID NO: 32 - protein, 11 aa, Llama
WGRGTQVTVSS

SEQ ID NO: 33 - protein, 121 aa, Llama
EVQLVESGGGLVQAGGSLRLSCAASGRTFAPGSMGWFRQAPGKEREFVAAHRWSGSAYYADYADSVEGRFTISRDYAKNMVYLQMNSLKPGDTAVYYCAAGVGSAAQYTYWGRGTQVTVSS

SEQ ID NO: 34 - protein, 5 aa, Llama
PGSMG

SEQ ID NO: 35 - protein, 19 aa, Llama
AHRWSGSAYYADYADSVEG

SEQ ID NO: 36 - protein, 10 aa, Llama
GVGSAAQYTY

SEQ ID NO: 37 - protein, 30 aa, Llama
EVQLVESGGGLVQAGGSLRLSCAASGRTFA

SEQ ID NO: 38 - protein, 14 aa, Llama
WFRQAPGKEREFVA

SEQ ID NO: 39 - protein, 32 aa, Llama
RFTISRDYAKNMVYLQMNSLKPGDTAVYYCAA

SEQ ID NO: 40 - protein, 11 aa, Llama
WGRGTQVTVSS

SEQ ID NO: 41 - protein, 121 aa, Llama
QVQLVESGGGLVQAGGSLRLSCAASGRTFGTYSMGWFRQAPGKEREFVAAHRWSGSAYYAEHADSVEGRFTISRDYAKNMLYLQMNSLKHEDTAVYYCAAGVGSEAQYRYWGRGTQVTVSS

SEQ ID NO: 42 - protein, 5 aa, Llama
TYSMG

SEQ ID NO: 43 - protein, 19 aa, Llama
AHRWSGSAYYAEHADSVEG

SEQ ID NO: 44 - protein, 10 aa, Llama
GVGSEAQYRY

SEQ ID NO: 45 - protein, 30 aa, Llama
QVQLVESGGGLVQAGGSLRLSCAASGRTFG

SEQ ID NO: 46 - protein, 14 aa, Llama
WFRQAPGKEREFVA

SEQ ID NO: 47 - protein, 32 aa, Llama; identical to SEQ ID NO: 31

SEQ ID NO: 48 - protein, 11 aa, Llama
WGRGTQVTVSS

SEQ ID NO: 49 - protein, 124 aa, Llama
QVQLVESGGGLVQAGDTLRLSCTASGRTFSSYSMGWFRQAPGKEREFVAAITWNGYITNYADSVKGRFTISRDNTKNTVFLQMNSLKPEETAVYYCAATTFSTTSPISRTYNYWGPGTQVTVSS

SEQ ID NO: 50 - protein, 5 aa, Llama
SYSMG

SEQ ID NO: 51 - protein, 17 aa, Llama
AITWNGYITNYADSVKG

SEQ ID NO: 52 - protein, 15 aa, Llama
TTFSTTSPISRTYNY

SEQ ID NO: 53 - protein, 30 aa, Llama
QVQLVESGGGLVQAGDTLRLSCTASGRTFS

SEQ ID NO: 54 - protein, 14 aa, Llama
WFRQAPGKEREFVA

SEQ ID NO: 55 - protein, 32 aa, Llama
RFTISRDNTKNTVFLQMNSLKPEETAVYYCAA

SEQ ID NO: 56 - protein, 11 aa, Llama
WGPGTQVTVSS

SEQ ID NO: 57 - protein, 120 aa, Llama
QVQLVESGGGLVQAGGSLRLSCVASSRTSSTYAMGWFRQGPGKERDFVAIISFGGTFYADSVKGRFTISRDNAKNTVYLQMNSLKPEDTAVYYCAAGRTLSKRADSYASWGQGTQVTVSS

SEQ ID NO: 58 - protein, 5 aa, Llama
TYAMG

SEQ ID NO: 59 - protein, 15 aa, Llama
IISFGGTFYADSVKG

SEQ ID NO: 60 - protein, 13 aa, Llama
GRTLSKRADSYAS

SEQ ID NO: 61 - protein, 30 aa, Llama
QVQLVESGGGLVQAGGSLRLSCVASSRTSS

SEQ ID NO: 62 - protein, 14 aa, Llama
WFRQGPGKERDFVA

SEQ ID NO: 63 - protein, 32 aa, Llama
RFTISRDNAKNTVYLQMNSLKPEDTAVYYCAA

SEQ ID NO: 64 - protein, 11 aa, Llama
WGQGTQVTVSS

SEQ ID NO: 65 - protein, 123 aa, Llama
EVQLVESGGGLVQAGGSLSLSCAASGGTLAMYAMSWFRQAPGKDRKFVAAINTSGRYSRYADSVKGRFTISRDNAKNTATLQMNSLEPEDTAVYYCAATDKGNWALAMSYDYWGQGTQVTVSS

SEQ ID NO: 66 - protein, 5 aa, Llama
MYAMS

SEQ ID NO: 67 - protein, 17 aa, Llama
AINTSGRYSRYADSVKG

SEQ ID NO: 68 - protein, 14 aa, Llama
TDKGNWALAMSYDY

SEQ ID NO: 69 - protein, 30 aa, Llama
EVQLVESGGGLVQAGGSLSLSCAASGGTLA

SEQ ID NO: 70 - protein, 14 aa, Llama
WFRQAPGKDRKFVA

SEQ ID NO: 71 - protein, 32 aa, Llama
RFTISRDNAKNTATLQMNSLEPEDTAVYYCAA

SEQ ID NO: 72 - protein, 11 aa, Llama
WGQGTQVTVSS

SEQ ID NO: 73 - protein, 16 aa, Artificial Sequence
  Heavy CDR2
  MISC_FEATURE (1)..(1): any amino acid
  MISC_FEATURE (3)..(3): any amino acid
XIXWSGRAPYADSVKG

SEQ ID NO: 74 - protein, 16 aa, Artificial Sequence
  Heavy CDR2
  MISC_FEATURE (1)..(1): Gly or Ala
  MISC_FEATURE (3)..(3): Ala or Ser
XIXWSGRAPYADSVKG

SEQ ID NO: 75 - protein, 15 aa, Artificial Sequence
  Heavy CDR3
  MISC_FEATURE (6)..(6): any amino acid
  MISC_FEATURE (10)..(10): any amino acid
  MISC_FEATURE (11)..(11): any amino acid
  MISC_FEATURE (14)..(14): any amino acid
GEGAIXWTTXXAYXY

SEQ ID NO: 76 - protein, 15 aa, Artificial Sequence
  Heavy CDR3
  MISC_FEATURE (6)..(6): Arg or Lys or Leu
  MISC_FEATURE (10)..(10): Leu or Pro
  MISC_FEATURE (11)..(11): Asp or Gly
  MISC_FEATURE (14)..(14): Asp or Asn
GEGAIXWTTXXAYXY

SEQ ID NO: 77 - protein, 5 aa, Artificial Sequence
  Heavy CDR1
  MISC_FEATURE (1)..(1): any amino acid
  MISC_FEATURE (2)..(2): any amino acid
XXSMG

SEQ ID NO: 78 - protein, 5 aa, Artificial Sequence
  Heavy CDR1
  MISC_FEATURE (1)..(1): Thr or Pro
  MISC_FEATURE (2)..(2): Tyr or Gly
XXSMG

SEQ ID NO: 79 - protein, 19 aa, Artificial Sequence
  Heavy CDR2
  MISC_FEATURE (12)..(12): any amino acid
  MISC_FEATURE (13)..(13): any amino acid
AHRWSGSAYYAXXADSVEG

SEQ ID NO: 80 - protein, 19 aa, Artificial Sequence
  Heavy CDR2
  MISC_FEATURE (12)..(12): Glu or Asp
  MISC_FEATURE (13)..(13): His or Tyr
AHRWSGSAYYAXXADSVEG

SEQ ID NO: 81 - protein, 10 aa, Artificial Sequence
  Heavy CDR3
  MISC_FEATURE (5)..(5): any amino acid
  MISC_FEATURE (9)..(9): any amino acid
GVGSXAQYXY

SEQ ID NO: 82 - protein, 10 aa, Artificial Sequence
  Heavy CDR3
  MISC_FEATURE (5)..(5): Ala or Glu
  MISC_FEATURE (9)..(9): Arg or Thr
GVGSXAQYXY

SEQ ID NO: 83 - protein, 122 aa, Llama
EVQLVESGGGLVQAGGSLRLSCAASGRTLSVYGTGWFRQAPGKEREFVAGISGTTGSTLYADSVKGRFTISRDNAKNTVYLQMNSLKSEDTALYYCAAGGRVYITTSSWAYWGQGTQVTVSS

SEQ ID NO: 84 - protein, 5 aa, Llama
VYGTG

SEQ ID NO: 85 - protein, 17 aa, Llama
GISGTTGSTLYADSVKG

SEQ ID NO: 86 - protein, 13 aa, Llama
GGRVYITTSSWAY

SEQ ID NO: 87 - protein, 30 aa, Llama
EVQLVESGGGLVQAGGSLRLSCAASGRTLS

SEQ ID NO: 88 - protein, 14 aa, Llama
WFRQAPGKEREFVA

SEQ ID NO: 89 - protein, 32 aa, Llama
RFTISRDNAKNTVYLQMNSLKSEDTALYYCAA

SEQ ID NO: 90 - protein, 11 aa, Llama
WGQGTQVTVSS

SEQ ID NO: 91 - protein, 127 aa, Llama
QLQLVESGGGLVQAGGSLRLSCVASGRTFSRYAMGWFRQAPGKEREFVAAIAWSTGSTYYANSVKGRFAISGDNAKNTVYLQMNSLKPEDTAVYYCAAETRYCSGFGCLDPRTYGSWGQGTQVTVSS

SEQ ID NO: 92 - protein, 5 aa, Llama
RYAMG

SEQ ID NO: 93 - protein, 17 aa, Llama
AIAWSTGSTYYANSVKG

SEQ ID NO: 94 - protein, 18 aa, Llama
ETRYCSGFGCLDPRTYGS

SEQ ID NO: 95 - protein, 30 aa, Llama
QLQLVESGGGLVQAGGSLRLSCVASGRTFS

SEQ ID NO: 96 - protein, 14 aa, Llama
WFRQAPGKEREFVA

SEQ ID NO: 97 - protein, 32 aa, Llama
RFAISGDNAKNTVYLQMNSLKPEDTAVYYCAA

SEQ ID NO: 98 - protein, 11 aa, Llama
WGQGTQVTVSS

SEQ ID NO: 99 - protein, 124 aa, Llama
QLQLVESGGGLVQPGGSLRLSCAASGRTFSTDTMAWFRQAPGKEREFIAGIGRSGGSIYYADAVKGRFTVSRDNAKNTVYLQMNSLKAEDTAVYYCAARQRIGLVVGALGYDYWGQGTQVTVSS

SEQ ID NO: 100 - protein, 5 aa, Llama
TDTMA

SEQ ID NO: 101 - protein, 17 aa, Llama
GIGRSGGSIYYADAVKG

SEQ ID NO: 102 - protein, 15 aa, Llama
RQRIGLVVGALGYDY

SEQ ID NO: 103 - protein, 30 aa, Llama
QLQLVESGGGLVQPGGSLRLSCAASGRTFS

SEQ ID NO: 104 - protein, 14 aa, Llama
WFRQAPGKEREFIA

SEQ ID NO: 105 - protein, 32 aa, Llama
RFTVSRDNAKNTVYLQMNSLKAEDTAVYYCAA

SEQ ID NO: 106 - protein, 11 aa, Llama
WGQGTQVTVSS

SEQ ID NO: 107 - protein, 129 aa, Llama
QLQLVESGGGLVQPGGSLRLSCAASGFTLDDYTIGWFRQAPGKEREGVSCINSITSNTYYADSVKGRFTISRDNAKNTVYLQMNSLTAEDTAIYYCAADSGLFSGSSCLKYRAMRFGSWGQGTQVTVSS

SEQ ID NO: 108 - protein, 5 aa, Llama
DYTIG

SEQ ID NO: 109 - protein, 17 aa, Llama
CINSITSNTYYADSVKG

SEQ ID NO: 110 - protein, 20 aa, Llama
DSGLFSGSSCLKYRAMRFGS

SEQ ID NO: 111 - protein, 30 aa, Llama
QLQLVESGGGLVQPGGSLRLSCAASGFTLD

SEQ ID NO: 112 - protein, 14 aa, Llama
WFRQAPGKEREGVS

SEQ ID NO: 113 - protein, 32 aa, Llama
RFTISRDNAKNTVYLQMNSLTAEDTAIYYCAA

SEQ ID NO: 114 - protein, 11 aa, Llama
WGQGTQVTVSS

SEQ ID NO: 115 - protein, 101 aa, Porcine
PRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCS

SEQ ID NO: 116 - protein, 1115 aa, Porcine
MDKLRMVLHENSGSADFRRCSAHLSSFTFAVVAVLSACLVTSSLGGKDKELRLTGGENKCSGRVEVKVQEEWGTVCNNGWDMDVVSVVCRQLGCPTAIKATGWANFSAGSGRIWMDHVSCRGNESALWDCKHDGWGKHNCTHQQDAGVTCSDGSDLEMGLVNGGNRCLGRIEVKFQGRWGTVCDDNFNINHASVVCKQLECGSAVSFSGSANFGEGSGPIWFDDLVCNGNESALWNCKHEGWGKHNCDHAEDAGVICLNGADLKLRVVDGVTECSGRLEVKFQGEWGTICDDGWDSDDAAVACKQLGCPTAVTAIGRVNASEGTGHIWLDSVSCHGHESALWQCRHHEWGKHYCNHDEDAGVTCSDGSDLELRLKGGGSHCAGTVEVEIQKLVGKVCDRSWGLKEADVVCRQLGCGSALKTSYQVYSKTKATNTWLFVSSCNGNETSLWDCKNWQWGGLSCDHYDEAKITCSAHRKPRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCSRYTQIRLVNGKTPCEGRVELNILGSWGSLCNSHWDMEDAHVLCQQLKCGVALSIPGGAPFGKGSEQVWRHMFHCTGTEKHMGDCSVTALGASLCSSGQVASVICSGNQSQTLSPCNSSSSDPSSSIISEENGVACIGSGQLRLVDGGGRCAGRVEVYHEGSWGTICDDSWDLNDAHVVCKQLSCGWAINATGSAHFGEGTGPIWLDEINCNGKESHIWQCHSHGWGRHNCRHKEDAGVICSEFMSLRLISENSRETCAGRLEVFYNGAWGSVGRNSMSPATVGVVCRQLGCADRGDISPASSDKTVSRHMWVDNVQCPKGPDTLWQCPSSPWKKRLASPSEETWITCANKIRLQEGNTNCSGRVEIWYGGSWGTVCDDSWDLEDAQVVCRQLGCGSALEAGKEAAFGQGTGPIWLNEVKCKGNETSLWDCPARSWGHSDCGHKEDAAVTCSEIAKSRESLHATGRSSFVALAIFGVILLACLIAFLIWTQKRRQRQRLSVFSGGENSVHQIQYREMNSCLKADETDMLNPSGDHSEVQ

SEQ ID NO: 117 - protein, 109 aa, Artificial Sequence
  His-tagged Porcine SRCR5
MPRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCSGHHHHHH

SEQ ID NO: 118 - protein, 143 aa, Artificial Sequence
  His-tagged VHH14 (02D01)
QLQLVESGGGLVQPGGSLRLSCAASGRTFSTDTMAWFRQAPGKEREFIAGIGRSGGSIYYADAVKGRFTVSRDNAKNTVYLQMNSLKAEDTAVYYCAARQRIGLVVGALGYDYWGQGTQVTVSSDYKDDDDKGGGGSHHHHHH